SEQUENCE LISTING (366 sequences)

SEQ ID NO: 1 - protein, 706 aa, Human gammaherpesvirus 4
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 2 - protein, 137 aa, Human gammaherpesvirus 4
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 3 - protein, 139 aa, synthetic construct
MDAMKRGLCCVLLLCGAVFVSPSASWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 4 - protein, 906 aa, Human gammaherpesvirus 4
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 5 - protein, 223 aa, Human gammaherpesvirus 4
MVSFKQVRVPLFTAIALVIVLLLAYFLPPRVRGGGRVAAAAITWVPKPNVEVWPVDPPPPVNFNKTAEQEYGDKEVKLPHWTPTLHTFQVPQNYTKANCTYCNTREYTFSYKGCCFYFTKKKHTWNGCFQACAELYPCTYFYGPTPDILPVVTRNLNAIESLWVGVYRVGEGNWTSLDGGTFKVYQIFGSHCTYVSKFSTVPVSHHECSFLKPCLCVSQRSNS

SEQ ID NO: 6 - protein, 902 aa, synthetic construct
MDAMKRGLCCVLLLCGAVFVSPSASSLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRTHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSYAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHGGGGSGGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGSHHHHHH

SEQ ID NO: 7 - protein, 1035 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 8 - protein, 1011 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 9 - protein, 986 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 10 - protein, 1037 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 11 - protein, 1013 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 12 - protein, 988 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 13 - protein, 1049 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 14 - protein, 1025 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 15 - protein, 1000 aa, synthetic construct
METDTLLLWVLLLWVPGSTGDYKDEQTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 16 - protein, 100 aa, synthetic construct
MGKSPTEVLLELIAEASGTTREEVKEKFLKELRKGKSPTEVLLELIAEASGTTKEEVKEKFLKELSFGKSPTEVLLELIAEASGTTKEEVKKKFWKELSL

SEQ ID NO: 17 - protein, 13 aa, synthetic construct
PTEVLLELIAEAS

SEQ ID NO: 18 - protein, 14 aa, synthetic construct
REEVKEKFLKELRK

SEQ ID NO: 19 - protein, 14 aa, synthetic construct
KEEVKEKFLKELSF

SEQ ID NO: 20 - protein, 14 aa, synthetic construct
KEEVKKKFWKELSL

SEQ ID NO: 21 - protein, 33 aa, synthetic construct
GKSPTEVLLELIAEASGTTREEVKEKFLKELRK

SEQ ID NO: 22 - protein, 33 aa, synthetic construct
GKSPTEVLLELIAEASGTTKEEVKEKFLKELSF

SEQ ID NO: 23 - protein, 33 aa, synthetic construct
GKSPTEVLLELIAEASGTTKEEVKKKFWKELSL

SEQ ID NO: 24 - protein, 199 aa, synthetic construct
MGDKTAIAQILAIKASAKGDETELERALRYAVKVGDKTAIAQILAIKASAKGDETELEQALRYAKFVGDKTAIAQILAIKASAKGDELELTRALAYAKKVGDKTAIAQILAIKASAKGDETELERALRYAKLVGDKTAIAQILAIKASAKGDETELERALRYAKYVGDKTAIAQILAIKASAKGDEPELEYALAYAKKV

SEQ ID NO: 25 - protein, 14 aa, synthetic construct
TAIAQILAIKASAK

SEQ ID NO: 26 - protein, 13 aa, synthetic construct
TELERALRYAVKV

SEQ ID NO: 27 - protein, 13 aa, synthetic construct
TELEQALRYAKFV

SEQ ID NO: 28 - protein, 13 aa, synthetic construct
LELTRALAYAKKV

SEQ ID NO: 29 - protein, 13 aa, synthetic construct
TELERALRYAKLV

SEQ ID NO: 30 - protein, 13 aa, synthetic construct
TELERALRYAKYV

SEQ ID NO: 31 - protein, 13 aa, synthetic construct
PELEYALAYAKKV

SEQ ID NO: 32 - protein, 33 aa, synthetic construct
GDKTAIAQILAIKASAKGDETELERALRYAVKV

SEQ ID NO: 33 - protein, 33 aa, synthetic construct
GDKTAIAQILAIKASAKGDETELEQALRYAKFV

SEQ ID NO: 34 - protein, 33 aa, synthetic construct
GDKTAIAQILAIKASAKGDELELTRALAYAKKV

SEQ ID NO: 35 - protein, 33 aa, synthetic construct
GDKTAIAQILAIKASAKGDETELERALRYAKLV

SEQ ID NO: 36 - protein, 33 aa, synthetic construct
GDKTAIAQILAIKASAKGDETELERALRYAKYV

SEQ ID NO: 37 - protein, 33 aa, synthetic construct
GDKTAIAQILAIKASAKGDEPELEYALAYAKKV

SEQ ID NO: 38 - protein, 221 aa, synthetic construct
LVSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLGVSLEQALKILKVAAELGTTVEEAVKRALKLKTKLGVSLEQALKILEVAAELGTTVEEAVKRAMKLKTKLGVSLEQALKILEVAAKLGTTVEEAVKRALKLKTKLGGWLEHHHHHH

SEQ ID NO: 39 - protein, 14 aa, synthetic construct
LEQALKILKVAAEL

SEQ ID NO: 40 - protein, 15 aa, synthetic construct
VEEAVKRALKLKTKL

SEQ ID NO: 41 - protein, 14 aa, synthetic construct
LEQALKILEVAAEL

SEQ ID NO: 42 - protein, 14 aa, synthetic construct
LEQALKILEVAAKL

SEQ ID NO: 43 - protein, 15 aa, synthetic construct
VEEAVKRAMKLKTKL

SEQ ID NO: 44 - protein, 35 aa, synthetic construct
LVSLEQALKILKVAAELGTTVEEAVKRALKLKTKL

SEQ ID NO: 45 - protein, 35 aa, synthetic construct
GVSLEQALKILKVAAELGTTVEEAVKRALKLKTKL

SEQ ID NO: 46 - protein, 35 aa, synthetic construct
GVSLEQALKILEVAAELGTTVEEAVKRALKLKTKL

SEQ ID NO: 47 - protein, 35 aa, synthetic construct
GVSLEQALKILEVAAKLGTTVEEAVKRALKLKTKL

SEQ ID NO: 48 - protein, 35 aa, synthetic construct
GVSLEQALKILEVAAELGTTVEEAVKRAMKLKTKL

SEQ ID NO: 49 - protein, 286 aa, synthetic construct
GSSMASGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLG

SEQ ID NO: 50 - protein, 379 aa, synthetic construct
GSSMASGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLG

SEQ ID NO: 51 - protein, 100 aa, synthetic construct
GSSMASGISVEELLKLAKAAYYSGTTVEEAYKLALKLGISVEELLKLAEAAYYSGTTVEEAYKLALKLGISVEELLKLAKAAYYSGTTVEEAYKLALKLG

SEQ ID NO: 52 - protein, 14 aa, synthetic construct
VEELLKLAKAAYYS

SEQ ID NO: 53 - protein, 11 aa, synthetic construct
VEEAYKLALKL

SEQ ID NO: 54 - protein, 14 aa, synthetic construct
VEELLKLAEAAYYS

SEQ ID NO: 55 - protein, 31 aa, synthetic construct
GISVEELLKLAKAAYYSGTTVEEAYKLALKL

SEQ ID NO: 56 - protein, 31 aa, synthetic construct
GISVEELLKLAEAAYYSGTTVEEAYKLALKL

SEQ ID NO: 57 - protein, 795 aa, synthetic construct
GLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGGW

SEQ ID NO: 58 - protein, 13 aa, synthetic construct
PEAIKAAAELGKA

SEQ ID NO: 59 - protein, 14 aa, synthetic construct
SEEILELLRAAHEL

SEQ ID NO: 60 - protein, 14 aa, synthetic construct
SEEILELLRAAHAL

SEQ ID NO: 61 - protein, 14 aa, synthetic construct
SAEILELLMAAHEL

SEQ ID NO: 62 - protein, 14 aa, synthetic construct
SEEILELLRAAHGL

SEQ ID NO: 63 - protein, 14 aa, synthetic construct
SEEILELLRAAHGG

SEQ ID NO: 64 - protein, 33 aa, synthetic construct
GLNPEAIKAAAELGKAGISSEEILELLRAAHEL

SEQ ID NO: 65 - protein, 33 aa, synthetic construct
GLNPEAIKAAAELGKAGISSEEILELLRAAHAL

SEQ ID NO: 66 - protein, 33 aa, synthetic construct
GLNPEAIKAAAELGKAGISSAEILELLMAAHEL

SEQ ID NO: 67 - protein, 33 aa, synthetic construct
GLNPEAIKAAAELGKAGISSEEILELLRAAHGL

SEQ ID NO: 68 - protein, 33 aa, synthetic construct
GLNPEAIKAAAELGKAGISSEEILELLRAAHGG

SEQ ID NO: 69 - protein, 271 aa, synthetic construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHVLGLDPEAIKAAAELGKAGISSEEILELLVAAHLLGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSIEILELLRAAHELGGW

SEQ ID NO: 70 - protein, 203 aa, synthetic construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELG

SEQ ID NO: 71 - protein, 137 aa, synthetic construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELG

SEQ ID NO: 72 - protein, 104 aa, synthetic construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHELG

SEQ ID NO: 73 - protein, 14 aa, synthetic construct
SEEILELLRAAHVL

SEQ ID NO: 74 - protein, 14 aa, synthetic construct
SIEILELLRAAHEL

SEQ ID NO: 75 - protein, 33 aa, synthetic construct
GLDPEAIKAAAELGKAGISSEEILELLRAAHEL

SEQ ID NO: 76 - protein, 33 aa, synthetic construct
GLDPEAIKAAAELGKAGISSEEILELLRAAHVL

SEQ ID NO: 77 - protein, 33 aa, synthetic construct
GLDPEAIKAAAELGKAGISSEEILELLVAAHLL

SEQ ID NO: 78 - protein, 33 aa, synthetic construct
GLDPEAIKAAAELGKAGISSIEILELLRAAHEL

SEQ ID NO: 79 - protein, 514 aa, synthetic construct
GNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEG

SEQ ID NO: 80 - protein, 24 aa, synthetic construct
ELALKALQILVNAAYVLAEIARDR

SEQ ID NO: 81 - protein, 27 aa, synthetic construct
ELLEKAARLAEEAARQAEEIARQARKE

SEQ ID NO: 82 - protein, 57 aa, synthetic construct
GNLELALKALQILVNAAYVLAEIARDRGNEELLEKAARLAEEAARQAEEIARQARKE

SEQ ID NO: 83 - protein, 93 aa, synthetic construct
GISVEELLKLAKAAYYSGTTVEEAYKLALKLGISVEELLKLAEAAYYSGTTVEEAYKLALKLGISVEELLKLAKAAYYSGTTVEEAYKLALKL

SEQ ID NO: 84 - protein, 14 aa, synthetic construct
SEEILELLVAAHLL

SEQ ID NO: 85 - protein, 14 aa, synthetic construct
SEEIKELLRAAHEL

SEQ ID NO: 86 - protein, 14 aa, synthetic construct
SEEILELLRAAHEK

SEQ ID NO: 87 - protein, 13 aa, synthetic construct
PEAEKAAAELGKA

SEQ ID NO: 88 - protein, 13 aa, synthetic construct
PEAIKKAAELGKA

SEQ ID NO: 89 - protein, 13 aa, synthetic construct
PECIKAAAELGKA

SEQ ID NO: 90 - protein, 13 aa, synthetic construct
PELEYALAYAKKV

SEQ ID NO: 91 - protein, 25 aa, synthetic construct
SELAARILIILFQQLVELARLAIES

SEQ ID NO: 92 - protein, 28 aa, synthetic construct
EELLRRVSEWLEEVIKDMRRVVEQALRE

SEQ ID NO: 93 - protein, 27 aa, synthetic construct
ELLRRVSEWLEEVIKDMRRVVEQALRE

SEQ ID NO: 94 - protein, 25 aa, synthetic construct
LELALKALQILVNAAYVLAEIARDR

SEQ ID NO: 95 - protein, 13 aa, synthetic construct
CEAIKAAAELGKA

SEQ ID NO: 96 - protein, 25 aa, synthetic construct
SELAARCLIILFQQLVELARLAIES

SEQ ID NO: 97 - protein, 13 aa, synthetic construct
CEAEKAAAELGKA

SEQ ID NO: 98 - protein, 13 aa, synthetic construct
CEAIKKAAELGKA

SEQ ID NO: 99 - protein, 13 aa, synthetic construct
CECIKAAAELGKA

SEQ ID NO: 100 - protein, 13 aa, synthetic construct
CTEVLLELIAEAS

SEQ ID NO: 101 - protein, 13 aa, synthetic construct
CELEYALAYAKKV

SEQ ID NO: 102 - skipped

SEQ ID NO: 103 - protein, 25 aa, synthetic construct
SELACRILIILFQQLVELARLAIES

SEQ ID NO: 104 - protein, 13 aa, synthetic construct
PECEKAAAELGKA

SEQ ID NO: 105 - protein, 13 aa, synthetic construct
PECIKKAAELGKA

SEQ ID NO: 106 - skipped

SEQ ID NO: 107 - protein, 16 aa, synthetic construct
GLDPEAIKAAAELGKA

SEQ ID NO: 108 - protein, 27 aa, synthetic construct
GNSELAARILIILFQQLVELARLAIES

SEQ ID NO: 109 - protein, 16 aa, synthetic construct
GDEPELEYALAYAKKV

SEQ ID NO: 110 - protein, 16 aa, synthetic construct
GLNPEAIKAAAELGKA

SEQ ID NO: 111 - protein, 16 aa, synthetic construct
GLDCEAIKAAAELGKA

SEQ ID NO: 112 - protein, 27 aa, synthetic construct
GNSELAARCLIILFQQLVELARLAIES

SEQ ID NO: 113 - protein, 16 aa, synthetic construct
GDECELEYALAYAKKV

SEQ ID NO: 114 - protein, 16 aa, synthetic construct
GLNCEAIKAAAELGKA

SEQ ID NO: 115 - protein, 16 aa, synthetic construct
GLNCEAEKAAAELGKA

SEQ ID NO: 116 - protein, 16 aa, synthetic construct
GLDCEAEKAAAELGKA

SEQ ID NO: 117 - protein, 16 aa, synthetic construct
GLDCEAIKKAAELGKA

SEQ ID NO: 118 - protein, 16 aa, synthetic construct
GLNCEAIKKAAELGKA

SEQ ID NO: 119 - protein, 16 aa, synthetic construct
GLDPECIKAAAELGKA

SEQ ID NO: 120 - protein, 27 aa, synthetic construct
GNSELACRILIILFQQLVELARLAIES

SEQ ID NO: 121 - protein, 16 aa, synthetic construct
GLNPECIKAAAELGKA

SEQ ID NO: 122 - protein, 27 aa, synthetic construct
GLNELCARILIILFQQLVELARLAIES

SEQ ID NO: 123 - protein, 27 aa, synthetic construct
GLNELCLKALQILVNAAYVLAEIARDR

SEQ ID NO: 124 - protein, 19 aa, synthetic construct
GLDPEAIKAAAELGKAGIS

SEQ ID NO: 125 - protein, 29 aa, synthetic construct
GNSELAARILIILFQQLVELARLAIESGD

SEQ ID NO: 126 - protein, 19 aa, synthetic construct
GKSPTEVLLELIAEASGTT

SEQ ID NO: 127 - protein, 19 aa, synthetic construct
GLNPEAIKAAAELGKAGIS

SEQ ID NO: 128 - protein, 19 aa, synthetic construct
GLDCEAIKAAAELGKAGIS

SEQ ID NO: 129 - protein, 29 aa, synthetic construct
GNSELAARCLIILFQQLVELARLAIESGD

SEQ ID NO: 130 - protein, 19 aa, synthetic construct
GKSCTEVLLELIAEASGTT

SEQ ID NO: 131 - protein, 19 aa, synthetic construct
GLNCEAIKAAAELGKAGIS

SEQ ID NO: 132 - protein, 19 aa, synthetic construct
GLNCEAEKAAAELGKAGTT

SEQ ID NO: 133 - protein, 19 aa, synthetic construct
GLDCEAEKAAAELGKAGIS

SEQ ID NO: 134 - protein, 19 aa, synthetic construct
GLDCEAIKKAAELGKAGTT

SEQ ID NO: 135 - protein, 19 aa, synthetic construct
GLDCEAIKKAAELGKAGIS

SEQ ID NO: 136 - protein, 19 aa, synthetic construct
GLDPECIKAAAELGKAGIS

SEQ ID NO: 137 - protein, 29 aa, synthetic construct
GNSELACRILIILFQQLVELARLAIESGD

SEQ ID NO: 138 - protein, 19 aa, synthetic construct
GLNPECIKAAAELGKAGIS

SEQ ID NO: 139 - protein, 57 aa, synthetic construct
GNSELAARILIILFQQLVELARLAIESGDEELLRRVSEWLEEVIKDMRRVVEQALRE

SEQ ID NO: 140 - protein, 33 aa, synthetic construct
GLDCEAIKAAAELGKAGISSEEILELLRAAHEL

SEQ ID NO: 141 - protein, 57 aa, synthetic construct
GNSELAARCLIILFQQLVELARLAIESGDEELLRRVSEWLEEVIKDMRRVVEQALRE

SEQ ID NO: 142 - protein, 33 aa, synthetic construct
GKSCTEVLLELIAEASGTTREEVKEKFLKELRK

SEQ ID NO: 143 - protein, 33 aa, synthetic construct
GKSCTEVLLELIAEASGTTKEEVKEKFLKELSF

SEQ ID NO: 144 - protein, 33 aa, synthetic construct
GKSCTEVLLELIAEASGTTKEEVKKKFWKELSL

SEQ ID NO: 145 - protein, 33 aa, synthetic construct
GLDCEAIKAAAELGKAGISSEEILELLRAAHVL

SEQ ID NO: 146 - protein, 33 aa, synthetic construct
GLDCEAIKAAAELGKAGISSEEILELLVAAHLL

SEQ ID NO: 147 - protein, 33 aa, synthetic construct
GLDCEAIKAAAELGKAGISSIEILELLRAAHEL

SEQ ID NO: 148 - protein, 33 aa, synthetic construct
GLNCEAIKAAAELGKAGISSEEILELLRAAHEL

SEQ ID NO: 149 - protein, 33 aa, synthetic construct
GLNCEAIKAAAELGKAGISSEEILELLRAAHAL

SEQ ID NO: 150 - protein, 33 aa, synthetic construct
GLNCEAIKAAAELGKAGISSAEILELLMAAHEL

SEQ ID NO: 151 - protein, 33 aa, synthetic construct
GLNCEAIKAAAELGKAGISSEEILELLRAAHGL

SEQ ID NO: 152 - protein, 33 aa, synthetic construct
GLNCEAIKAAAELGKAGISSEEILELLRAAHGG

SEQ ID NO: 153 - protein, 33 aa, synthetic construct
GLDPECIKAAAELGKAGISSEEILELLRAAHEL

SEQ ID NO: 154 - protein, 57 aa, synthetic construct
GNSELACRILIILFQQLVELARLAIESGDEELLRRVSEWLEEVIKDMRRVVEQALRE

SEQ ID NO: 155 - protein, 33 aa, synthetic construct
GLDPECIKAAAELGKAGISSEEILELLRAAHVL

SEQ ID NO: 156 - protein, 33 aa, synthetic construct
GLDPECIKAAAELGKAGISSEEILELLVAAHLL

SEQ ID NO: 157 - protein, 33 aa, synthetic construct
GLDPECIKAAAELGKAGISSIEILELLRAAHEL

SEQ ID NO: 158 - protein, 33 aa, synthetic construct
GLNPECIKAAAELGKAGISSEEILELLRAAHEL

SEQ ID NO: 159 - protein, 33 aa, synthetic construct
GLNPECIKAAAELGKAGISSEEILELLRAAHAL

SEQ ID NO: 160 - protein, 33 aa, synthetic construct
GLNPECIKAAAELGKAGISSAEILELLMAAHEL

SEQ ID NO: 161 - protein, 33 aa, synthetic construct
GLNPECIKAAAELGKAGISSEEILELLRAAHGL

SEQ ID NO: 162 - protein, 33 aa, synthetic construct
GLNPECIKAAAELGKAGISSEEILELLRAAHGG

SEQ ID NO: 163 - DNA, 2454 bp, synthetic construct
  source 1..2454: mol_type = other DNA
atggctagcagccatcatcatcatcatcatagcagcggcctggtgccgcgcggcagctccatgggcctggacccggaagcgatcaaagcggctgcggaactgggcaaagctggtatctcgtcagaagaaatcctggaactgctgcgtgcggcgcatgaactgggcctggatccggaagccattaaagccgcggcggaactgggcaaagcgggtatcagcagcgaagaaatcctggaactgctgcgcgcggcccatgaactgggtctggatccggaagccatcaaagccgcggcggaactgggcaaagcgggcatcagcagcgaagaaatcctggaactgctgcgcgccgcgcatgaactgggtctggatccggaagcgattaaagccgccgccgaactgggtaaagcgggtattagcagcgaagaaatcctggaactgctgcgtgcggcgcatgaactgggcctggatccggaagccatcaaagccgccgccgaactgggtaaagccggtatcagcagcgaagaaattctggaactgctgcgtgccgcccatgaactgggcctggatccggaagcgattaaagccgccgcggaactgggtaaagccggtattagcagcgaagaaatcctggaactgctgcgtgccgcgcatgaactgggtctggatccggaagccatcaaagcggcggccgaactgggtaaagccggcatcagcagcgaagaaatcctggaactgctgcgtgccgcccatgaactgggcctggatccggaagcgatcaaagccgccgcggaactgggtaaagccggcattagcagcgaagaaatcctggaactgctgcgtgccgcgcatgaactgggtctggatccggaagcgattaaagcggcggccgaactgggcaaagccggtattagcagcgaagaaatcctggaactgctgcgtgcggcccatgaactgggcctggatccggaagcgatcaaagcggcggccgaactgggtaaagcgggtatcagcagcgaagaaatcctggaactgctgcgtgccgcccatgaactgggtctggatccggaagccattaaagcggcggcggaactgggtaaagcgggcatcagcagcgaagaaattctggaactgctgcgcgcggcgcatgaactgggtctggatccggaagccattaaagccgccgcggaactgggcaaagcgggtattagcagcgaagaaattctggaactgctgcgcgcggcgcatgaactgggcctggatccggaagcgattaaagccgccgccgaactgggcaaagccggcatcagcagcgaagaaattctggaactgctgcgcgccgcccatgaactgggcctggatccggaagcgatcaaagccgccgcggaactgggcaaagcgggcatcagcagcgaagaaatcctggaactgctgcgtgcggcgcatgaactgggtctggatccggaagccattaaagccgcggcggaactgggcaaagccggcattagcagcgaagaaattctggaactgctgcgtgcggcgcatgaactgggtctggatccggaagccatcaaagccgcggcggaactgggtaaagcgggtattagcagcgaagaaattctggaactgctgcgcgcggcccatgaactgggcctggatccggaagcgattaaagcggcggccgaactgggcaaagcgggcattagcagcgaagaaatcctggaactgctgcgcgcggcgcatgaactgggtctggatccggaagccattaaagccgccgccgaactgggcaaagccggtatcagcagcgaagaaattctggaactgctgcgcgcggcccatgaactgggtctggatccggaagccatcaaagccgccgcggaactgggtaaagccggtatcagcagcgaagaaatcctggaactgctgcgcgccgcccatgaactgggcctggatccggaagcgatcaaagccgcggcggaactgggtaaagcgggcattagcagcgaagaaattctggaactgctgcgtgcggcccatgaactgggtctggatccggaagcgatcaaagccgcggccgaactgggtaaagcgggcatcagcagcgaagaaattctggaactgctgcgtgccgcgcatgaactgggcctggatccggaagcgattaaagccgcggccgaactgggcaaagcgggcattagcagcgaagaaattctggaactgctgcgcgcggcgcatgaactgggcctggatccggaagcgattaaagcggcggccgaactgggcaaagcgggtatcagcagcgaagaaatcctggaactgctgcgcgccgcgcatgaactgggcctggatccggaagcgattaaagccgcggccgaactgggcaaagcgggtatctcgtctgaagaaatcctggaactgctgcgtgctgcccatgaactgggtggttggtaataa

SEQ ID NO: 164 - protein, 816 aa, synthetic construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 165 - DNA, 372 bp, synthetic construct
  source 1..372: mol_type = other DNA
atggctagcagccatcatcatcatcatcatagcagcggcctggtgccgcgcggcagctccatgggcctggatccggaagctattaaagcggcggcagaactgggcaaagccggtattagctctgaagaaatcctggaactgctgcgtgctgcgcatgaactgggtctggacccggaagcaatcaaagccgcagctgaactgggcaaagcgggtattagttccgaagaaattctggaactgctgcgtgccgcacatgaactgggtctggatccggaagccattaaagcagctgcggaactgggtaaagccggtattagcagcgaggagattctggaactgctgcgcgccgcacatgaactgggcggttggtaa

SEQ ID NO: 166 - protein, 123 aa, synthetic construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 167 - DNA, 471 bp, synthetic construct
  source 1..471: mol_type = other DNA
atggctagcagccatcatcatcatcatcatagcagcggcctggtgccgcgcggcagctccatgggcctggatccggaagcgattaaagcggcggcagaactgggcaaagccggtattagctctgaagaaatcctggaactgctgcgtgctgcgcatgaactgggtctggacccggaagcaatcaaagccgcagctgaactgggcaaagctggtattagttccgaagaaattctggaactgctgcgtgccgcacatgaactgggtctggatccggaagccatcaaagcagctgcggaactgggtaaagcgggtatttcatcggaggagatcctggaactgctgcgcgcggcccacgaactgggcctggatccggaagctatcaaagctgcggccgaactgggtaaagctggtattagcagcgaggagattctggaactgctgcgcgcagctcacgaactgggcggttggtaa

SEQ ID NO: 168 - protein, 156 aa, synthetic construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 169 - DNA, 669 bp, synthetic construct
  source 1..669: mol_type = other DNA
atggctagcagccatcatcatcatcatcatagcagcggcctggtgccgcgcggcagctccatgggcctggatccggaagcaattaaagcggcggcagaactgggcaaagctggtattagctctgaagaaatcctggaactgctgcgtgctgcgcatgaactgggtctggacccggaagcaatcaaagccgcagctgaactgggcaaagcgggtattagttccgaagaaattctggaactgctgcgtgccgcacatgaactgggtctggatccggaagccattaaagcagctgcggaactgggcaaagcaggtatttcatcggaggagatcctggaactgctgcgcgcggcccacgaactgggcctggacccggaagcgatcaaagctgcggccgaactgggcaaagccggcatctctagcgaggagattctggaactgctgcgcgcagctcacgaactgggcctggatccggaagcgatcaaggcggcggcagagctgggcaaagccggtattagttcagaggagatactggaactgctgcgcgccgcccacgagttaggtctggatccggaagcgattaaggccgcagctgagctgggtaaagccggtattagctccgaggagatattagaactgctgcgtgccgcgcatgaactgggcggttggtaa

SEQ ID NO: 170 - protein, 222 aa, synthetic construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 171 - protein, 261 aa, synthetic construct
METDTLLLWVLLLWVPGSTGDYKDEGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 172 - DNA, 867 bp, synthetic construct
  source 1..867: mol_type = other DNA
atggctagcagccatcatcatcatcatcatagcagcggcctggtgccgcgcggcagctccatgggcctggatccggaagcgattaaagcggcggcagaactgggcaaagccggtattagctctgaagaaatcctggaactgctgcgtgctgcgcatgaactgggtctggacccggaagcaatcaaagccgcagctgaactgggcaaagctggtattagttccgaagaaattctggaactgctgcgtgccgcacatgaactgggtctggatccggaagcaattaaagcagctgcggaactgggcaaagcgggtatttcatcggaggagatcctggaactgctgcgcgccgcacatgtgctgggcctggatccggaagccatcaaagctgcggccgaactgggcaaagcaggcatctctagcgaggagattctggaactgctggttgcagctcacctgctgggtttagaccctgaagccatcaaggcggcggcagagctgggtaaagcgggcatcagttccgaggagatactggaactgctgcgcgcggcccacgaactgggcctggacccggaagcgattaaggccgcagctgagctgggtaaagcgggtatcagctcggaggagatattagaactgctgcgcgccgcccatgagttaggcctggacccggaagccatcaaggcagctgcggagctgggtaaagctggcatctctagcgaggaaattttagagctgctgcgtgcggcccacgagttaggtctggacccggaagccattaaggctgcggccgagctgggcaaagcgggtatctcatccattgagatattggagctgctgcgtgcagctcacgaactgggcggttggtaa

SEQ ID NO: 173 - protein, 288 aa, synthetic construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHVLGLDPEAIKAAAELGKAGISSEEILELLVAAHLLGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSIEILELLRAAHELGGW

SEQ ID NO: 174 - protein, 327 aa, synthetic construct
METDTLLLWVLLLWVPGSTGDYKDEGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 175 - DNA, 1266 bp, synthetic construct
  source 1..1266: mol_type = other DNA
atggctagcagccatcatcatcatcatcatagcagcggcctggtgccgcgcggcagctccatgggcctggacccggaggcgatcaaggctgcggcggaactgggtaaagcgggcatcagcagcgaggaaattctggagctgctgcgtgcggcgcatgagctgggtctggacccggaagcgattaaggctgcggcggagctgggcaaggcgggtatcagcagcgaagaaattctggaactgctgcgcgcggcgcatgagctgggcctggacccggaagcgatcaaagctgcggcggagctgggtaaggctggtatcagcagcgaggagattctggaattattacgcgcggcgcatgaactgggtctggacccggaggcgattaaagctgcggcggaactgggcaaggcgggcatcagcagcgaagagattctggaattacttcgtgctgcgcatgaactgggcctggacccggaggctattaaggctgcggcggaattaggcaaagctggcatcagcagcgaagaaattctggaattactgcgtgccgctcatgaattaggtctggacccggaagctatcaaagctgcggcggaattaggtaaggccggtatcagcagcgaggaaattctggaattgttacgagctgctcatgaattaggcctggacccggaggccattaaggctgcggcggagttaggcaaagcgggtatcagcagcgaggaaattctggaacttttacgagctgctcatgagttaggtctggacccggaggctatcaaggctgcggcggagttgggcaaagccggcatcagcagcgaggagattctggaacttttacgggctgctcatgaacttggcctggacccggaggcaattaaggctgcggcggagctaggcaaggctggtatcagcagcgaggaaattctggaactactgcgtgccgcccatgaattgggtctggacccggaagcaatcaaggctgcggcggagcttggcaaggccggtatcagcagcgaggaaattctggaactgttacgagctgcccatgaacttggtctggacccggaagccataaaagctgcggcggagttaggtaaggcaggtatcagcagcgaggaaattctggaactcctgcgtgccgcacatgaactcggtctggacccggaggccattaaagctgcggcggagttgggtaaggcgggtatcagcagcgaggaaattctggagttactgcgtgccgcgcatgagctgggtggctggtaataa

SEQ ID NO: 176 - protein, 420 aa, synthetic construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 177 - DNA, 1266 bp, synthetic construct
  source 1..1266: mol_type = other DNA
atggctagcagccatcatcatcatcatcatagcagcggcctggtgccgcgcggcagctccatgggcctggactgcgaggcgatcaaggctgcggcggaactgggtaaagcgggcatcagcagcgaggaaattctggagctgctgcgtgcggcgcatgagctgggtctggacccggaagcgattaaggctgcggcggagctgggcaaggcgggtatcagcagcgaagaaattctggaactgctgcgcgcggcgcatgagctgggcctggacccggaagcgatcaaagctgcggcggagctgggtaaggctggtatcagcagcgaggagattctggaattattacgcgcggcgcatgaactgggtctggacccggaggcgattaaagctgcggcggaactgggcaaggcgggcatcagcagcgaagagattctggaattacttcgtgctgcgcatgaactgggcctggacccggaggctattaaggctgcggcggaattaggcaaagctggcatcagcagcgaagaaattctggaattactgcgtgccgctcatgaattaggtctggacccggaagctatcaaagctgcggcggaattaggtaaggccggtatcagcagcgaggaaattctggaattgttacgagctgctcatgaattaggcctggacccggaggccattaaggctgcggcggagttaggcaaagcgggtatcagcagcgaggaaattctggaacttttacgagctgctcatgagttaggtctggacccggaggctatcaaggctgcggcggagttgggcaaagccggcatcagcagcgaggagattctggaacttttacgggctgctcatgaacttggcctggacccggaggcaattaaggctgcggcggagctaggcaaggctggtatcagcagcgaggaaattctggaactactgcgtgccgcccatgaattgggtctggacccggaagcaatcaaggctgcggcggagcttggcaaggccggtatcagcagcgaggaaattctggaactgttacgagctgcccatgaacttggtctggacccggaagccataaaagctgcggcggagttaggtaaggcaggtatcagcagcgaggaaattctggaactcctgcgtgccgcacatgaactcggtctggacccggaatgcataaaggctgcggcggaattgggcaaagctggtatcagcagcgaggaaattctggagttactgcgtgccgcgcatgagctgggtggctggtaataa

SEQ ID NO: 178 - protein, 420 aa, synthetic construct
MASSHHHHHHSSGLVPRGSSMGLDCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGGW

SEQ ID NO: 179 - DNA, 1266 bp, synthetic construct
  source 1..1266: mol_type = other DNA
atggctagcagccatcatcatcatcatcatagcagcggcctggtgccgcgcggcagctccatgggcctggacccggaggcggaaaaggctgcggcggagctgggtaaagcgggcatcagcagcgaggaaattaaagaactgctgcgtgcggcgcatgagctgggtctggacccggaagcgatcaaagctgcggcggaactgggcaaggcgggtatcagcagcgaagaaattctggaactgctgcgcgcggcgcatgagctgggcctggacccggaagcgattaaagctgcggcggagctgggcaaggcgggcatcagcagcgaagagattctggaattattacgcgcggcgcatgaactgggtctggacccggaggcgatcaaggctgcggcggaactgggtaaggctggtatcagcagcgaggagattctggaattacttcgtgctgcgcatgaactgggcctggacccggaggcgattaaggctgcggcggagttaggcaaagctggcatcagcagcgaagaaattctggaattactgcgtgccgctcatgaattaggtctggacccggaagctatcaaggctgcggcggagttgggcaaagccggcatcagcagcgaggagattctggaacttttacgagctgctcatgaattaggcctggacccggaagctattaaagctgcggcggaattaggtaaggccggtatcagcagcgaggaaattctggaattgttacgagctgctcatgagttaggtctggacccggaggctatcaaagctgcggcggagttaggtaaggcaggtatcagcagcgaggaaattctggaacttttacgggctgctcatgaacttggcctggacccggaagccatcaaagctgcggcggaattagggaaagctggaatcagcagcgaagagattctggaacttcttcgtgccgctcatgaacttggtctggacccggaggccattaaagctgcggcggaacttggtaaggcgggtatcagcagcgaggaaattctggaactactgcgtgccgcccatgaattgggtctggacccggaagcaatcaaggctgcggcggagcttggcaaagcaggtatcagcagcgaggaaattctggagttattgcgtgccgctcatgaactcggtctggacccggaagccatcaagaaagcggcggaactgggcaaagcaggcatcagcagcgaagagattctggagctgctgcgtgcggcgcacgaaaaaggtggctggtaataa

SEQ ID NO: 180 - protein, 420 aa, synthetic construct
MASSHHHHHHSSGLVPRGSSMGLDPEAEKAAAELGKAGISSEEIKELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKKAAELGKAGISSEEILELLRAAHEKGGW

SEQ ID NO: 181 - DNA, 669 bp, synthetic construct
  source 1..669: mol_type = other DNA
atggctagcagccatcatcatcatcatcatagcagcggcctggtgccgcgcggcagctccatgggcctggactgcgaggccattaaggctgcggcggagttaggcaaagcgggtatcagcagcgaggaaattctggaacttttacgagctgctcatgagttaggtctggacccggaggctatcaaggctgcggcggagttgggcaaagccggcatcagcagcgaggagattctggaacttttacgggctgctcatgaacttggcctggacccggaggcaattaaggctgcggcggagctaggcaaggctggtatcagcagcgaggaaattctggaactactgcgtgccgcccatgaattgggtctggacccggaagcaatcaaggctgcggcggagcttggcaaggccggtatcagcagcgaggaaattctggaactgttacgagctgcccatgaacttggtctggacccggaagccataaaagctgcggcggagttaggtaaggcaggtatcagcagcgaggaaattctggaactcctgcgtgccgcacatgaactcggtctggacccggagtgcattaaagctgcggcggagttgggtaaggcgggtatcagcagcgaggaaattctggagttactgcgtgccgcgcatgagctgggtggctggtaa

SEQ ID NO: 182 - protein, 222 aa, synthetic construct
MASSHHHHHHSSGLVPRGSSMGLDCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGGW

SEQ ID NO: 183 - protein, 172 aa, synthetic construct
GNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEG

SEQ ID NO: 184 - protein, 342 aa, synthetic construct
GNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALRE

SEQ ID NO: 185 - protein, 457 aa, synthetic construct
GNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREG

SEQ ID NO: 186 - protein, 457 aa, synthetic construct
GNSELAARCLIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELACRILIILFQQLVELARLAIESGDEELLRRVSEWLEEVIKDMRRVVEQALREG

SEQ ID NO: 187 - protein, 99 aa, synthetic construct
GKSPTEALLKLIAEAKGITETEAKEEAEKALKEGKSPTEALLKLIAEAKGITETEAKEEAEKALKEGKSPTEALLKLIAEAKGITETEAKEEAEKALKE

SEQ ID NO: 188 - protein, 99 aa, synthetic construct
GKSPTEALLKLIAEAKGITSTEAKEEAIKALKEGKSPTEALLKLIAEAKGITELEAKVLAEKALKEGKSPTEALLKLIAEAKGITETEAKLEAEKALKE

SEQ ID NO: 189 - protein, 99 aa, synthetic construct
GKSPTEVLLELIAEASGTTKEEVKEKFLKELSKGKSPTEVLLELIAEASGTTKEEVKEKFLKELSKGKSPTEVLLELIAEASGTTKEEVKEKFLKELSK

SEQ ID NO: 190 - protein, 99 aa, synthetic construct
GKSPTEVLLELIAEASGTTKEEVKRKFLKELSKGKSPTEVLLELIAEASGTTKAEVKREFLWELSLGKSPTEVLLELIAEASGTTKEEVKEKFLAELEK

SEQ ID NO: 191 - protein, 99 aa, synthetic construct
GKSPTEVLLELIAEASGTTREEVKEKFLKELRKGKSPTEVLLELIAEASGTTKEEVKEKFLKELSFGKSPTEVLLELIAEASGTTKEEVKKKFWKELSL

SEQ ID NO: 192 - protein, 99 aa, synthetic construct
GKSPTEVLLELIAEASGTTKEEVKEKFLKELSKGKSPTEVLLELIAEASGTTKEEVKEKFLKELSKGKSPTEVLLELIAEASGTTKREVKRWFLFELRK

SEQ ID NO: 193 - protein, 99 aa, synthetic construct
GKSPTEVLLELIAEASGTTKAEVKLKFLFELSFGKSPTEVLLELIAEASGTTKEEVKEKFLKELFKGKSPTEVLLELIAEASGTTKEEVKEKFLKELSK

SEQ ID NO: 194 - protein, 99 aa, synthetic construct
GYSTTEALLILIAEASGTTVEQQKQRFKELVKKGYSTTEALLILIAEASGTTVEQQKQRFKELVKKGYSTTEALLILIAEASGTTVEQQKQRFKELVKK

SEQ ID NO: 195 - protein, 198 aa, synthetic construct
GDKTAIAQILAIKASAKGDETELERALRYAKKVGDKTAIAQILAIKASAKGDETELERALRYAKKVGDKTAIAQILAIKASAKGDETELERALRYAKKVGDKTAIAQILAIKASAKGDETELERALRYAKKVGDKTAIAQILAIKASAKGDETELERALRYAKKVGDKTAIAQILAIKASAKGDETELERALRYAKKV

SEQ ID NO: 196 - protein, 198 aa, synthetic construct
GDKTAIAQILAIKASAKGDETELERALRYAVKVGDKTAIAQILAIKASAKGDETELEQALRYAKFVGDKTAIAQILAIKASAKGDELELTRALAYAKKVGDKTAIAQILAIKASAKGDETELERALRYAKLVGDKTAIAQILAIKASAKGDETELERALRYAKYVGDKTAIAQILAIKASAKGDEPELEYALAYAKKV

SEQ ID NO: 197 - protein, 198 aa, synthetic construct
GDKTAIAQILAIKASAKGDETELERALRYAKKVGDKTAIAQILAIKASAKGDETELERALIFAEAVGDKTAIAQILAIKASAKGDETELERALRYAKKVNDKTAIAQILAIKASAKGDETELDRALWYAKKVGDKTAIAQILAIKASAKGDETELERALRYAKKVGDKTAIAQILAIKASAKGDETELERALLYAKKV

SEQ ID NO: 198 - protein, 198 aa, synthetic construct
GDKTAIAQILAIKASAKGDETELERALRYAKKVGDKTAIAQILAIKASAKGDETELERALAYARLVGDKTAIAQILAIKASAKGDETELERALRYAEKVGDKTAIAQILAIKASAKGDEQELEAALIYAKKVGDKTAIAQILAIKASAKGDETELERALRYAKKVGDKTAIAQILAIKASAKGDETELERALWYAKKV

SEQ ID NO: 199 - protein, 198 aa, synthetic construct
GDRSAIATAYIALAEYLGDKEALLKAIEIAIKLGDRSAIAEAYIALARYLGDKEALLKAIEIAIKLGDRSAIATAYIALAEYLGDKEALLKAIEIAIKLGDRSAIAEAYIALARYLGDKEALLKAIEIAIKLGDRSAIATAYIALAEYLGDKEALLKAIEIAIKLGDRSAIAEAYIALARYLGDKEALLKAIEIAIKL

SEQ ID NO: 200 - protein, 198 aa, synthetic construct
GDKSALAQILAIYASAYGDTTLFLRALKLAKEVGDKSALAQILAIYASAYGDTTLFLRALKLAKEVGDKSALAQILAIYASAYGDTTLFLRALKLAKEVGDKSALAQILAIYASAYGDTTLFLRALKLAKEVGDKSALAQILAIYASAYGDTTLFLRALKLAKEVGDKSALAQILAIYASAYGDTTLFLRALKLAKEV

SEQ ID NO: 201 - protein, 198 aa, synthetic construct
GDLELYIRVLAIVAEAEGDKTKLELALKLALKKGDLKLYIEVLAIVAEAEGDKTKLELALKLALKKGDLELYIRVLAIVAKAEGDKTKLELALKLALKKGDLKLYIEVLAIVAEAEGDKTKLELALKLALKKGDLELYIRVLAIVAEAEGDKTKLELALKLALKKGDLKLYIEVLAIVAKAEGDKTKLELALKLALKK

SEQ ID NO: 202 - protein, 210 aa, synthetic construct
VSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLGVSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLG

SEQ ID NO: 203 - protein, 210 aa, synthetic construct
VSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLGVSLEQALKILKVAAELGTTVEEAVKRALKLKTKLGVSLEQALKILEVAAELGTTVEEAVKRAMKLKTKLGVSLEQALKILEVAAKLGTTVEEAVKRALKLKTKLG

SEQ ID NO: 204 - protein, 279 aa, synthetic construct
GISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKL

SEQ ID NO: 205 - protein, 372 aa, synthetic construct
GISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKL

SEQ ID NO: 206 - protein, 13 aa, synthetic construct
PTEALLKLIAEAK

SEQ ID NO: 207 - protein, 14 aa, synthetic construct
ETEAKEEAEKALKE

SEQ ID NO: 208 - protein, 14 aa, synthetic construct
STEAKEEAIKALKE

SEQ ID NO: 209 - protein, 14 aa, synthetic construct
ELEAKVLAEKALKE

SEQ ID NO: 210 - protein, 14 aa, synthetic construct
ETEAKLEAEKALKE

SEQ ID NO: 211 - protein, 14 aa, synthetic construct
KEEVKEKFLKELSK

SEQ ID NO: 212 - protein, 14 aa, synthetic construct
KEEVKRKFLKELSK

SEQ ID NO: 213 - protein, 14 aa, synthetic construct
KAEVKREFLWELSL

SEQ ID NO: 214 - protein, 14 aa, synthetic construct
KEEVKEKFLAELEK

SEQ ID NO: 215 - protein, 14 aa, synthetic construct
KREVKRWFLFELRK

SEQ ID NO: 216 - protein, 14 aa, synthetic construct
KAEVKLKFLFELSF

SEQ ID NO: 217 - protein, 14 aa, synthetic construct
KEEVKEKFLKELFK

SEQ ID NO: 218 - protein, 13 aa, synthetic construct
TTEALLILIAEAS

SEQ ID NO: 219 - protein, 14 aa, synthetic construct
VEQQKQRFKELVKK

SEQ ID NO: 220 - protein, 13 aa, synthetic construct
TELERALRYAKKV

SEQ ID NO: 221 - protein, 13 aa, synthetic construct
TELERALIFAEAV

SEQ ID NO: 222 - protein, 13 aa, synthetic construct
TELDRALWYAKKV

SEQ ID NO: 223 - protein, 13 aa, synthetic construct
TELERALLYAKKV

SEQ ID NO: 224 - protein, 13 aa, synthetic construct
TELERALAYARLV

SEQ ID NO: 225 - protein, 13 aa, synthetic construct
TELERALRYAEKV

SEQ ID NO: 226 - protein, 13 aa, synthetic construct
TELERALWYAKKV

SEQ ID NO: 227 - protein, 14 aa, synthetic construct
SAIATAYIALAEYL

SEQ ID NO: 228 - protein, 13 aa, synthetic construct
EALLKAIEIAIKL

SEQ ID NO: 229 - protein, 14 aa, synthetic construct
SAIAEAYIALARYL

SEQ ID NO: 230 - protein, 14 aa, synthetic construct
SALAQILAIYASAY

SEQ ID NO: 231 - protein, 13 aa, synthetic construct
TLFLRALKLAKEV

SEQ ID NO: 232 - protein, 14 aa, synthetic construct
ELYIRVLAIVAEAE

SEQ ID NO: 233 - protein, 13 aa, synthetic construct
TKLELALKLALKK

SEQ ID NO: 234 - protein, 14 aa, synthetic construct
KLYIEVLAIVAEAE

SEQ ID NO: 235 - protein, 14 aa, synthetic construct
ELYIRVLAIVAKAE

SEQ ID NO: 236 - protein, 14 aa, synthetic construct
KLYIEVLAIVAKAE

SEQ ID NO: 237-238 - skipped (2 entries)

SEQ ID NO: 239 - protein, 33 aa, synthetic construct
GKSPTEALLKLIAEAKGITETEAKEEAEKALKE

SEQ ID NO: 240 - protein, 33 aa, synthetic construct
GKSPTEALLKLIAEAKGITSTEAKEEAIKALKE

SEQ ID NO: 241 - protein, 33 aa, synthetic construct
GKSPTEALLKLIAEAKGITELEAKVLAEKALKE

SEQ ID NO: 242 - protein, 33 aa, synthetic construct
GKSPTEALLKLIAEAKGITETEAKLEAEKALKE

SEQ ID NO: 243 - protein, 33 aa, synthetic construct
GKSPTEVLLELIAEASGTTKEEVKEKFLKELSK

SEQ ID NO: 244 - protein, 33 aa, synthetic construct
GKSPTEVLLELIAEASGTTKEEVKRKFLKELSK

SEQ ID NO: 245 - protein, 33 aa, synthetic construct
GKSPTEVLLELIAEASGTTKAEVKREFLWELSL

SEQ ID NO: 246 - protein, 33 aa, synthetic construct
GKSPTEVLLELIAEASGTTKEEVKEKFLAELEK

SEQ ID NO: 247 - protein, 33 aa, synthetic construct
GKSPTEVLLELIAEASGTTKREVKRWFLFELRK

SEQ ID NO: 248 - protein, 33 aa, synthetic construct
GKSPTEVLLELIAEASGTTKAEVKLKFLFELSF

SEQ ID NO: 249 - protein, 33 aa, synthetic construct
GKSPTEVLLELIAEASGTTKEEVKEKFLKELFK

SEQ ID NO: 250 - protein, 33 aa, synthetic construct
GYSTTEALLILIAEASGTTVEQQKQRFKELVKK

SEQ ID NO: 251 - protein, 33 aa, synthetic construct
GDKTAIAQILAIKASAKGDETELERALRYAKKV

SEQ ID NO: 252 - protein, 33 aa, synthetic construct
GDKTAIAQILAIKASAKGDETELERALIFAEAV

SEQ ID NO: 253 - protein, 33 aa, synthetic construct
NDKTAIAQILAIKASAKGDETELDRALWYAKKV

SEQ ID NO: 254 - protein, 33 aa, synthetic construct
GDKTAIAQILAIKASAKGDETELERALLYAKKV

SEQ ID NO: 255 - protein, 33 aa, synthetic construct
GDKTAIAQILAIKASAKGDETELERALAYARLV

SEQ ID NO: 256 - protein, 33 aa, synthetic construct
GDKTAIAQILAIKASAKGDETELERALRYAEKV

SEQ ID NO: 257 - protein, 33 aa, synthetic construct
GDKTAIAQILAIKASAKGDEQELEAALIYAKKV

SEQ ID NO: 258 - protein, 33 aa, synthetic construct
GDKTAIAQILAIKASAKGDETELERALWYAKKV

SEQ ID NO: 259 - protein, 33 aa, synthetic construct
GDRSAIATAYIALAEYLGDKEALLKAIEIAIKL

SEQ ID NO: 260 - protein, 33 aa, synthetic construct
GDRSAIAEAYIALARYLGDKEALLKAIEIAIKL

SEQ ID NO: 261 - protein, 33 aa, synthetic construct
GDKSALAQILAIYASAYGDTTLFLRALKLAKEV

SEQ ID NO: 262 - protein, 33 aa, synthetic construct
GDLELYIRVLAIVAEAEGDKTKLELALKLALKK

SEQ ID NO: 263 - protein, 33 aa, synthetic construct
GDLKLYIEVLAIVAEAEGDKTKLELALKLALKK

SEQ ID NO: 264 - protein, 33 aa, synthetic construct
GDLELYIRVLAIVAKAEGDKTKLELALKLALKK

SEQ ID NO: 265 - protein, 33 aa, synthetic construct
GDLKLYIEVLAIVAKAEGDKTKLELALKLALKK

SEQ ID NO: 266 - protein, 11 aa, synthetic construct
GGSGGGSGGSG

SEQ ID NO: 267 - protein, 10 aa, synthetic construct
GGSGGGSGSG

SEQ ID NO: 268 - protein, 8 aa, synthetic construct
GGSGGGSG

SEQ ID NO: 269 - protein, 10 aa, synthetic construct
GGGGSGGGGS

SEQ ID NO: 270 - protein, 8 aa, synthetic construct
GGGSGGGS

SEQ ID NO: 271 - protein, 6 aa, synthetic construct
GGSGGS

SEQ ID NO: 272 - protein, 15 aa, synthetic construct
GLNDIFEAQKIEWHE

SEQ ID NO: 273 - protein, 6 aa, synthetic construct
HHHHHH

SEQ ID NO: 274 - protein, 4 aa, synthetic construct
LPHD

SEQ ID NO: 275 - protein, 4 aa, synthetic construct
NPND

SEQ ID NO: 276 - protein, 4 aa, synthetic construct
DPKD

SEQ ID NO: 277 - protein, 5 aa, synthetic construct
GLEPD

SEQ ID NO: 278 - protein, 5 aa, synthetic construct
GVSLD

SEQ ID NO: 279 - protein, 5 aa, synthetic construct
GVLPD

SEQ ID NO: 280 - protein, 25 aa, synthetic construct
MDAMKRGLCCVLLLCGAVFVSPSAS

SEQ ID NO: 281 - protein, 114 aa, synthetic construct
WAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 282 - protein, 661 aa, synthetic construct
SLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRTHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSYAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAH

SEQ ID NO: 283 - protein, 198 aa, synthetic construct
CEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLG

SEQ ID NO: 284 - protein, 20 aa, synthetic construct
METDTLLLWVLLLWVPGSTG

SEQ ID NO: 285 - protein, 5 aa, synthetic construct
DYKDE

SEQ ID NO: 286 - protein, 710 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNP

SEQ ID NO: 287 - protein, 686 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSIT

SEQ ID NO: 288 - protein, 661 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVS

SEQ ID NO: 289 - protein, 686 aa, synthetic construct
MDAMKRGLCCVLLLCGAVFVSPSASSLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRTHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSYAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAH

SEQ ID NO: 290 - protein, 16 aa, synthetic construct
GGGSGGGSGGGSGGGS

SEQ ID NO: 291 - protein, 263 aa, synthetic construct
CEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGL

SEQ ID NO: 292 - protein, 4 aa, synthetic construct
GGGS

SEQ ID NO: 293 - protein, 7 aa, synthetic construct
ENLYFQS

SEQ ID NO: 294 - protein, 877 aa, synthetic construct
SLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRTHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSYAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHGGGGSGGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGSHHHHHH

SEQ ID NO: 295 - protein, 1010 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 296 - protein, 986 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 297 - protein, 961 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 298 - protein, 1012 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 299 - protein, 988 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 300 - protein, 963 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 301 - protein, 1024 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 302 - protein, 1000 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 303 - protein, 975 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 304 - protein, 871 aa, synthetic construct
SLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRTHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSYAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHGGGGSGGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGS

SEQ ID NO: 305 - protein, 979 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 306 - protein, 955 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 307 - protein, 930 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 308 - protein, 981 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 309 - protein, 957 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 310 - protein, 932 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 311 - protein, 993 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 312 - protein, 969 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 313 - protein, 944 aa, synthetic construct
QTPEQPAPPATTVQPTATRQQTSFPFRVCELSSHGDLFRFSSDIQCPSFGTRENHTEGLLMVFKDNIIPYSFKVRSYTKIVTNILIYNGHRADSVTNRHEEKFSVDSYETDQMDTIYQCYNAVKMTKDGLTRVYVDRDGVNITVNLKPTGGLANGVRRYASQTELYDAPGRVEATYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDVGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 314 - protein, 183 aa, synthetic construct
GGGGSGGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGSHHHHHH

SEQ ID NO: 315 - protein, 58 aa, synthetic construct
GSSKKQGDADVCGEVAYIQSVVSDCHVPTAELRTLLEIRKLFLEIQKLKVELQGLSKE

SEQ ID NO: 316 - protein, 189 aa, synthetic construct
GGGGSGGGGSGGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 317 - protein, 8 aa, synthetic construct
DLYDDDDK

SEQ ID NO: 318 - protein, 26 aa, synthetic construct
KRRWKKNFIAVSAANRFKKISSSGAL

SEQ ID NO: 319 - protein, 9 aa, synthetic construct
YPYDVPDYA

SEQ ID NO: 320 - protein, 10 aa, synthetic construct
EQKLISEEDL

SEQ ID NO: 321 - protein, 8 aa, synthetic construct
WRHPQFGG

SEQ ID NO: 322 - protein, 8 aa, synthetic construct
WSHPQFEK

SEQ ID NO: 323 - protein, 13 aa, synthetic construct
SLAELLNAGLGGS

SEQ ID NO: 324 - protein, 8 aa, synthetic construct
TQDPSRVG

SEQ ID NO: 325 - protein, 14 aa, synthetic construct
GKPIPNPLLGLDST

SEQ ID NO: 326 - protein, 7 aa, synthetic construct
ENLYFQG

SEQ ID NO: 327 - protein, 7 aa, synthetic construct
ENLYFQS

SEQ ID NO: 328 - protein, 7 aa, synthetic construct
ETVRFQS

SEQ ID NO: 329 - protein, 7 aa, synthetic construct
ETVRFQG

SEQ ID NO: 330 - protein, 68 aa, synthetic construct
SGRAHAGWETPEGCEQVLTGKRLMQCLPNPEDVKMALEVYKLSLEIEQLELQRDSARQSTLDKELVPR

SEQ ID NO: 331 - protein, 55 aa, synthetic construct
KKQGDADVCGEVAYIQSVVSDCHVPTAELRTLLEIRKLFLEIQKLKVELQGLSKE

SEQ ID NO: 332 - protein, 57 aa, synthetic construct
ETPEGCEQVLTGKRLMQCLPNPEDVKMALEVYKLSLEIEQLELQRDSARQSTLDKEL

SEQ ID NO: 333 - protein, 58 aa, synthetic construct
WETPEGCEQVLTGKRLMQCLPNPEDVKMALEVYKLSLEIEQLELQRDSARQSTLDKEL

SEQ ID NO: 334 - protein, 52 aa, synthetic construct
CEQVLTGKRLMQCLPNPEDVKMALEVYKLSLEIEQLELQRDSARQSTLDKEL

SEQ ID NO: 335 - protein, 57 aa, synthetic construct
ETPEGCEQVLTGKRLMQCLPNPEDVKMALEVYKLSLEIEQLELQRDSARQYTLDKEL

SEQ ID NO: 336 - protein, 57 aa, synthetic construct
ETPEGCEQVLAGKRLMQCLPNPEDVKMALEVYKLSLEIEQLELQRDRARQSTLDKEL

SEQ ID NO: 337 - protein, 57 aa, synthetic construct
ETPEGCEQVLAGKRLMQCLPNPEDVKMALEVYKLSLEIEQLELQRDRARQSTWDKEL

SEQ ID NO: 338 - protein, 57 aa, synthetic construct
EVPEGCEQVQAGRRLMQCLADPYEVKMALEVYKLSLEIELLELQRDKARKSSVLRQL

SEQ ID NO: 339 - protein, 57 aa, synthetic construct
VVPEGCEHILKGRKTMQCLPNPEDVKMALEIYKLSLDIELLELQRDRAKESTVQSPV

SEQ ID NO: 340 - protein, 55 aa, synthetic construct
EVPKDCEHVFAGKKLMQCLPNSNDVKMALEVYKLTLEIKQLQLQIDKAKHVDREL

SEQ ID NO: 341 - protein, 57 aa, synthetic construct
EYPEDCEQVHEGKKLMQCLPNLEEIKLALELYKLSLETKLLELQIDKEKKAKAKYSI

SEQ ID NO: 342 - protein, 57 aa, synthetic construct
EYPEDCEQVHEGKKLMECLPTLEEIKLALALYKLSLETNLLELQIDKEKKAKAKYST

SEQ ID NO: 343 - protein, 57 aa, synthetic construct
EIAEGCEQVLAGRKIMQCLPKPEDVRTALELYKLSLEIKQLEKKLEKEEKCTPEVQE

SEQ ID NO: 344 - protein, 67 aa, synthetic construct
EYPEGCEQVVTGRKLLQCLSRPEEVKLALEVYKLSLEIEILQTNKLKKEAFLLREREKNVTCDFNPE

SEQ ID NO: 345 - protein, 52 aa, synthetic construct
EYPEGCEQVVTGRKLLKCLSRPEEVKLALEVYKLSLEIALLELQIDKPKDAS

SEQ ID NO: 346 - protein, 52 aa, synthetic construct
EVPENCEQVIVGKKLMKCLSNPDEAQMALQLYKLSLEAELLRLQIVKARQGS

SEQ ID NO: 347 - protein, 54 aa, synthetic construct
EASEDLKPALTGNKTMQYVPNSHDVKMALEIYKLTLEVELLQLQIQKEKHTEAH

SEQ ID NO: 348 - protein, 51 aa, synthetic construct
VSAEVCEAVFKGQKLLKCLPNAMEVKMALEVYKLSLEIEKLEQEKRKLEIA

SEQ ID NO: 349 - protein, 66 aa, synthetic construct
EVPEECKQVAAGRKLLECLPNPSDVKMALEVYKLSLEIEQLEKEKYVKIQEKFSKKEMKQLTSALH

SEQ ID NO: 350 - protein, 59 aa, synthetic construct
EVLEDCRIVSRGAQLLHCLSSPEDVHRALKVYKLFLEIERLEHQKEKWIQLHRKPQSMK

SEQ ID NO: 351 - protein, 63 aa, synthetic construct
EGPEDCEIVNKGRQLLQCLSSPEDVQRALEVYKLSLEIERLEQQREKRTSVHRKAHYTKVDGP

SEQ ID NO: 352 - protein, 67 aa, synthetic construct
EAPEGCEQVLTGRKLMQCLPSPEDVKVALEVYKLSLEIKQLEKERDKLMNTHQKFSEKEEMKDLFFP

SEQ ID NO: 353 - protein, 52 aa, synthetic construct
EVPEGCEQVLTGKKLMQCLPNPEDVKMALEVYKLSLEIELLELQIDKARQGS

SEQ ID NO: 354 - protein, 47 aa, synthetic construct
GCEQVLTGKRLMQCLPNPEDVKMALEVYKLSLEIEQLELQRDSARQS

SEQ ID NO: 355 - protein, 51 aa, synthetic construct
ETPEGCEQVLTGKRLMQCLPNPEDVKMALEVYKLSLEIEQLELQRDSARQS

SEQ ID NO: 356 - protein, 53 aa, synthetic construct
GSEQVLTGKRLMQSLPNPEDVKMALEVYKLSLEIEQLELQRDSARQSTLDKEL

SEQ ID NO: 357 - protein, 57 aa, synthetic construct
ETPEGCEQVLTGKRLMQCLPNPEDVKMALEIYKLTLEIEQLELQRDSARQSTLDKEL

SEQ ID NO: 358 - protein, 57 aa, synthetic construct
ETPEGCEQVLTGKRLMQCLPNPEDVKMALEIYKLSLEIKQLELQRDSARQSTLDKEL

SEQ ID NO: 359 - protein, 50 aa, synthetic construct
EGCEQILTGKRLMQCLPDPEDVKMALEIYKLSLEIKQLELQRDRARQSTL

SEQ ID NO: 360 - protein, 57 aa, synthetic construct
ETPEGCEQVLTGKRLMQCLPNPEDVKMALEVYKLSLEIKQLELQRDRARQSTLDKEL

SEQ ID NO: 361 - protein, 52 aa, synthetic construct
EGCEQILTGKRLMQCLPNPEDVKMALEIYKLSLEIEQLELQRDRARQSTLDK

SEQ ID NO: 362 - protein, 61 aa, synthetic construct
WETPEGCEQVLTGKRLMQCLPNPEDVKMALEVYKLSLEIEQLELQRDSARQSTLDKELVPR

SEQ ID NO: 363 - protein, 4 aa, synthetic construct
AELR

SEQ ID NO: 364 - protein, 62 aa, Homo sapiens
AHAGWETPEGCEQVLTGKRLMQCLPNPEDVKMALEVYKLSLEIEQLELQRDSARQSTLDKEL

SEQ ID NO: 365 - protein, 56 aa, Gallus gallus
SKKQGDADVCGEVAYIQSVVSDCHVPTEDVKTLLEIRKLFLEIQKLKVELQGLSKE

SEQ ID NO: 366 - protein, 56 aa, synthetic construct
SKKQGDADVCGEVAYIQSVVSDCHVPTAELRTLLEIRKLFLEIQKLKVELQGLSKE

(19) United States
(12) Patent Application Publication
(10) Pub. No.: US 2024/0091346 A1
(43) Pub. Date: Mar. 21, 2024

(54) EPSTEIN BARR VIRUS VACCINES

(71) Applicant: Fred Hutchinson Cancer Center, Seattle, WA (US)

(72) Inventors: Jason Price, Kent, WA (US); James M. Olson, Seattle, WA (US); Andrew McGuire, Seattle, WA (US); Barry L. Stoddard, Seattle, WA (US)

(73) Assignee: Fred Hutchinson Cancer Center, Seattle, WA (US)

(21) Appl. No.: 18/458,108

(22) Filed: Aug. 29, 2023

Related U.S. Application Data
(60) Provisional application No. 63/373,789, filed on Aug. 29, 2022.

(51) Int. Cl. A61K 39/245; A61P 31/22; C12N 7/00

(52) U.S. Cl. CPC A61K 39/245; A61P 31/22; C12N 7/00; A61K 2039/55

(57) ABSTRACT

Anti-Epstein Barr Virus (EBV) vaccines are described herein. The EBV vaccine antigens are linked to a multimerization domain, for example, presented on a circular tandem repeat protein (cTRP) scaffold or linked to a C4b multimerization domain. The vaccines can be used to treat and/or reduce the risk of EBV infection and to treat and/or reduce the risk of complications associated with EBV infection, such as infectious mononucleosis, lymphoproliferative disorders, carcinomas, and smooth muscle tumors.

### CROSS REFERENCE TO RELATED APPLICATION

This application claims priority to U.S. Provisional Patent Application No. 63/373,789, filed on Aug. 29, 2022, the contents of which are incorporated herein by reference in their entirety.

### FIELD OF THE DISCLOSURE

Anti-Epstein Barr Virus (EBV) vaccines and uses of the same are described. The vaccine antigens are linked to a multimerization domain, for example presented on a circular tandem repeat protein (cTRP) scaffold or linked to a C4b multimerization domain. The multimerized vaccine antigens can be used to treat and/or reduce the risk of EBV infection and also to treat and/or reduce the risk of complications associated with EBV infection, such as infectious mononucleosis, lymphoproliferative disorders, carcinomas, and smooth muscle tumors.

### REFERENCE TO SEQUENCE LISTING

The Sequence Listing associated with this application is provided in XML format in lieu of a paper copy and is hereby incorporated by reference into the specification. The name of the file containing the Sequence Listing is 2YD9502-Sequence Listing.xml. The file is 376,046 bytes, was created on Aug. 29, 2023, and is being submitted electronically via Patent Center.

### BACKGROUND OF THE DISCLOSURE

Epstein Barr virus (EBV) is an orally transmitted gamma herpesvirus that infects B cells and epithelial cells in the majority of adults worldwide. Most primary infections are asymptomatic, however, EBV is a causative agent of infectious mononucleosis (IM) in children and young adults. Following primary infection, infected individuals become lifelong carriers of EBV, which can lie dormant (i.e., latent) in cells. However, in certain individuals the latent virus can begin to express genes that alter cellular replication, leading to cancer or cancer-like diseases. EBV is also associated with nasopharyngeal carcinoma and lymphoproliferative disorders in immunocompromised patients such as those with HIV/AIDS or in patients undergoing immune suppression for organ transplantation. Thus, vaccines that treat, reduce, or prevent EBV infection of cells or treat EBV-associated diseases would be a major benefit to public health.

A primary goal of most vaccine design strategies is to elicit production of neutralizing antibodies, which are a type of antibody that can inhibit the biological function of its target. Neutralizing antibodies against viruses such as EBV typically function by blocking a virus from entering a cell.

To enter a cell, EBV, like other herpesviruses, first attaches to the cell surface through an interaction between a protein on the surface of the virus and a receptor binding site of a cell surface protein. Following this attachment, the virus membrane can fuse with the cell membrane, allowing the contents of the virus to be inserted into the cell. Viral fusion also occurs through interaction of a viral protein with an epitope on an antigen of a cell protein. The interactions resulting in viral attachment to a cell and the interactions resulting in viral fusion to a cell are distinct, each involving different viral proteins and different cellular proteins. Thus, neutralizing antibodies could block EBV entry into cells by preventing virus/cell protein interactions leading to attachment and/or fusion.

Many previous efforts to design EBV vaccines have included vaccines that target the EBV protein gp350, which is involved in EBV attachment to cells. However, while a phase 2 clinical trial showed that the vaccine could reduce the incidence of IM, it did not protect from EBV infection.

### SUMMARY OF THE DISCLOSURE

The current disclosure provides anti-Epstein Barr virus (EBV) vaccines and uses of the same. The vaccine antigens are linked to a multimerization domain, for example, presented on a circular tandem repeat protein (cTRP) scaffold or linked to a C4b multimerization domain. The multimerized vaccine antigens can be used to treat and/or reduce the risk of EBV infection and also to treat and/or reduce the risk of complications associated with EBV infection, such as infectious mononucleosis, lymphoproliferative disorders, carcinomas, and smooth muscle tumors.

In particular embodiments the EBV vaccine antigens can be formulated to treat an EBV-infected subject or a subject at risk of EBV infection. Treating EBV can reduce EBV infection and/or treat a condition associated with EBV infection, such as infectious mononucleosis or lymphoproliferative disorder. Moreover, high levels of antibodies that neutralize EBV B-cell infection are associated with lowered risks for nasopharyngeal carcinoma.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A-1C. (1A) HSV-1 gB pre-fusion and post-fusion structures. (1B) cTRP (Toroid) scaffold. (1C) cTRP scaffolded gB Model. Rationale: Ideal geometry between cTRPs and gB

FIG. 2. Linker length variants and EBV gB ectodomain variants.

FIGS. 3A-3K. (3A) Exemplary EBV antigen proteins and signal peptides; (3B) Antigen Fragments; (3C) EBV gH/gL heterodimer cTRP sequences and EBV gB cTRP sequences; (3D) EBV Vaccine Sequences including signal peptides, tags, and cleavage sites; (3E) EBV Vaccine Sequences without signal peptide and without flag tag; (3F) EBV vaccine antigen sequences linked to cTRP sequence.

FIG. 4. EBV gB-cTRPs—Superose 6 SEC post crude Ni-purification.

FIG. 5. Purified products by SDS page and HPLC SEC.

FIG. 6. EM screening for candidate prefusion structures.

FIG. 7. Viral glycoprotein scaffolding: EBV gH/GI gH/gL-cTRP Tetramer-Model. Top, bottom, and side views.

FIG. 8. Production: NiNTA, S200 SEC Purification.

FIG. 9. gH/gL-cTRP raw NS EM data.

FIG. 10. Depiction of antigen presentation on the “top” and “bottom” of a cTRP protein scaffold.

FIG. 11. Table of yields of various gH/gL nanoparticles. See FIGS. 12A-12-H.

FIGS. 12A-12H. Biochemical and Biophysical Characterization of multimeric gH/gL nanoparticles. (12A) Monomeric gH/gL and multimeric gH/gL nanoparticles were analyzed by size-exclusion chromatography (SEC) on a Superose 6 column as indicated. (12B) Reducing SDS-PAGE analysis of 1 μg of monomeric gH/gL or multimeric gH/gL nanoparticles. Bands corresponding to gL, gH, and gH fused to 4-mer, 7-mer, or 24-mer multimerization domains (MD) are indicated with arrows. (12C) Non-Reducing SDS-PAGE analysis of 1 μg of the proteins in FIG. 12B. (12D) Negative stain electron microscopy was performed on 4-mer, 7-mer, or 24-mer gH/gL nanoparticles as indicated. The eight most frequent 2D class averages for each particle are shown in the inlay. Scale bars represent 200 nm. Binding of the anti-gH/gL mAbs E1D1, CL40, CL59 and AMMO1 to monomeric gH/gL (12E) or multimeric gH/gL nanoparticles (12F-12H) were measured by ELISA as indicated. Each data point represents the mean and error bars represent the standard deviation of two technical replicates. The anti-HIV-1 Env mAb VRCO1 was used as a control for non-specific binding. See also FIGS. 11 and 13.

FIG. 13. Table of multimerization domains and observed and expected nanoparticle sizes. See FIGS. 12A-12H. *Includes the weight of the peptide component predicted by https://web.expasy.org/protparam/plus the weight of 8 putative N-linked glycosylation sites on gH/gL, each assigned a molecular weight of 1 kDa. **the column used for size exclusion chromatography appears in parentheses.

FIG. 14A-14D. Immunogenicity of gH/gL nanoparticles. (14A) C57BL/6 mice (n=10 mice for gH/gL monomer, 4-mer, 7-mer, and 24-mer) were immunized with monomeric gH/gL or multimeric gH/gL nanoparticles at weeks 0, 4, and 12. Blood was collected 2 weeks after each immunization. (14B) Endpoint plasma binding titers to gH/gL were measured by ELISA. Each dot represents the reciprocal endpoint titer for an individual mouse measured in duplicate. Box and whisker plots represent the minimum, 25th percentile, median, 75th percentile, and maximum values. The ability of plasma from individual mice to neutralize EBV infection of epithelial cells (14C), or B cells (14D). Each dot represents the reciprocal half-maximal inhibitory dilution (ID50) titer of an individual mouse. Plasma that did not achieve 50% neutralization at the lowest dilution tested (1:20) was assigned a value of 10. Box and whisker plots represent the minimum, 25th percentile, median, 75th percentile, and maximum values. Significant differences were determined using Mann-Whitney tests with Holm-adjusted p-values (*p<0.05, **p<0.01, ***p<0.001). See also FIGS. 15 and 16.

FIG. 15. Inhibition of EBV infection of epithelial cells by gH/gL immune plasma. Plasma from C57BL/6 (n=10-12 per group) mice were serially diluted and evaluated for their ability to inhibit AKATA-GFP EBV infection of SVKCR2 cells. Animals immunized with the same monomeric gH/gL or gH/gL nanoparticles are bound by boxes. The y-axis shows the % of background subtracted GFP+SVKCR2 cells and the y-axis is the plasma dilution. Each data point represents mean and error bars represent the standard deviation of two technical replicates. Different colored symbols represent the same individual mouse in each group at the indicated timepoints. The dashed line indicates the % of GFP+SVKCR2 cells in the absence of plasma. See FIGS. 14A-14D.

FIG. 16. Inhibition of EBV infection in B cells by gH/gL immune plasma. Plasma from C57BL/6 mice (n=10-12 per group) were serially diluted and evaluated for their ability to inhibit EBV B95.8/F infection of Raji cells. Animals immunized with the same monomeric gH/gL or gH/gL nanoparticles are bound by boxes. The y-axis shows the % of background subtracted GFP+Raji cells and the y-axis is the plasma dilution. Each data point represents mean and the error bars represent the standard deviation of two technical replicates. Different colored symbols represent the same individual moue in each group at the indicated timepoints. The dashed line indicates the % of GFP+Raji cells in the absence of plasma. See FIGS. 14A-14D.

FIG. 17A-17D. Plasma competition against monoclonal anti-gH/gL antibodies. The ability of plasma pooled from groups of mice immunized with monomeric gH/gL or multimeric gH/gL nanoparticles to inhibit binding to a panel of anti-gH/gL antibodies to monomeric gH/gL was measured by ELISA. The heatmap depicts the log reciprocal plasma dilution titers resulting in a 50% inhibition of (17A) E1D1, (17B) CL40, (17C) CL59, or (17D) AMMO1 antibodies at each time point. See FIGS. 18A-18C for titration curves.

FIG. 18A-18C. Competitive binding between immune plasma and monoclonal antibodies. Competitive binding ELISAs were performed using pools of plasma from groups of C57BL/6 mice (n=10-12 per group) immunized with monomeric gH/gL or multimeric gH/gL nanoparticles, and a panel of anti-gH/gL antibodies. At each time point, pooled sera from each group were titrated on to gH/gL immobilized on an ELISA plate, after which either AMMO1, CL40, CL59, or E1D1 antibodies were added at a concentration previously determined to achieve half maximal binding. Competitions were performed using plasma pools collected at Post-1st (18A), Post-2^{nd} (18B), and Post-3rd timepoints (18C). Each data point represents mean and error bars represent the standard deviation of two technical replicates. See FIGS. 17A-17D.

FIG. 19A-19L. Depletion of AMMO1-KO insensitive antibodies from pooled plasma. The binding of AMMO1 (19A), CL40 (19B), CL59 (19C), and E1D1 (19D) binding to gH/gL and gH/gL-KO (gH K73W, Y76A/gL) were measured using biolayer interferometry. (19E-19H) Antibodies were depleted from pooled plasma collected following three immunizations with gH/gL or gH/gL nanoparticles using gH/gL-KO conjugated magnetic beads. Pre- and post-depletion plasma samples were assayed for binding to gH/gL and gH/gL-KO by ELISA as indicated. Each data point represents mean and error bars represent the standard deviation of two technical replicates. (19I-19L) The ability of plasma pre- and post-depletion to neutralize EBV infection was measured in B cells and epithelial cells. Each data point represents the mean and error bars represent the standard deviation of two technical replicates.

FIGS. 20A-20G. Negative stain electron microscopy (EM) was performed to assess pre vs post fusion structures in the candidate designs for (20A) MDT-001296 (EBV_gB_EctoM_mut-GS-cTRP (8)ss-TEV-HisAvi), (20B) MDT-001297 (EBV_gB_EctoS_mut-GS-cTRP(8)ss-TEV-HisAvi), (20C) MDT-001298 (EBV_gB_EctoL_mut-GGGS-cTRP(8)ss-TEV-HisAvi), (20D) MDT-001299 (EBV_gB_EctoM_mut-GGGS-cTRP(8)ss-TEV-HisAvi), (20E) MDT-001300 (EBV_gB_EctoS_mut-GGGS-cTRP(8)ss-TEV-HisAvi), (20F) MDT-001302 (EBV_gB_EctoM_mut-GGGS(4)-cTRP(8)ss-TEV-HisAvi), and (20G) MDT-001303 (EBV_gB_EctoS_mut-GGGS(4)-cTRP(8)ss-TEV-HisAvi). MDT-001301 (EBV gB EctoL mut-GGGS (4)-cTRP(8)ss-TEV-HisAvi) did not result in any micrographs and for the aggregated sample, no apparent particles were on the grid. (20B) MDT-001297 and (20C) MDT-001298 are the only constructs that generated molecules with dimensions consistent with anticipated prefusion structure. Fitting of related prefusion gB structures and cTRP model structure revealed that MDT-1297 produced a structure consistent with this modeling.

FIG. 21. Fitting pre and post-fusion structures into the generated maps and prioritization of MDT-001297 was performed. EBV gB post-fusion structure with cTRP model and related hCMV gB prefusion structure with toroid model fit into MDT-001297 map. Fits suggest that MDT-001297 production includes both pre and post-fusion states. Structures were fit to maps using ChimeraX program. Other designed constructs did not form prefusion states suggesting that combination of linker length and selection of gB C-terminal truncation site are important for stabilization for prefusion state.

FIG. 22. Additional sequences supporting the disclosure including exemplary cTRP scaffolds creating alpha helices and circular and closed (also referred to as “stapled”) architectures. The following nomenclature can be used to reference specific cTRPs: “dTor_(number of structural repeats)×(number of amino acid residues within each structural repeat)(protein handedness)”. For example, “dTor_3×33L” describes a cTRP protein including a single protein chain containing 3 repeats of 33 repetitive amino acids, wherein the helical bundles of that cTRP are entirely left-handed. In the case that a cTRP is assembled from multiple protein subunits that each contain a fraction of the total repeats in that cTRP, the nomenclature also may indicate the number of repeats in each subunit of a multimeric assemblage, and thereby can distinguish between multimeric cTRPs that contain the same total number of repeats. For example, “dTor_12×31L_sub3” describes an assembly of 4 identical protein subunits that each have 3 left-handed repeats, that come together to create a multimeric cTRP with 12 total repeats. In contrast, “dTor_12×31L sub4” describes an assembly of 3 identical protein subunits that each have 4 repeats, that again come together to create a multimeric cTRP with 12 total repeats. Within FIG. 22, Group I provides sequences that form alpha-helical segments with N-terminal or C-terminal mutation positions to form a stapled cTRP bolded and underlined (sequences with no potential mutation positions or no naturally occurring cysteines at position 1 or 3 are not included in the 1st alpha helix of N-terminal or C-terminal segments (SEQ ID NOs: 59, 139, 84, 140, 85-88, 58, 89, 17, 90, 60-63, and 18-20); FIG. 22 Group II provides thick cTRP sequences that form alpha-helical segments with N-terminal or C-terminal mutation n positions at 5 and 7 bolded and underlined (SEQ ID NOs: 91-93, 80, 81, and 94); FIG. 22 Group III provides modified N-terminal segments (SEQ ID NOs: 95-101); FIG. 22 Group IV provides modified C-terminal segments (SEQ ID NOs: 89, 99, 103-105); FIG. 22 Group V provides sequences with N-terminal segment mutation positions and/or C-terminal segment mutation positions underlined and bolded with leading linker sequences (SEQ ID NOs: 107-110); FIG. 22 Group VI provides sequences with N-terminal segment mutations with leading linker sequences (SEQ ID NOs: 111-114, 115, 116, 133, and 118); FIG. 22 Group VII provides sequences with C-terminal segment mutations with leading linker sequences (SEQ ID NOs: 119-123); FIG. 22 Group VIII provides sequences with N-terminal segment mutation positions and/or C-terminal segment mutation positions underlined and bolded with leading and following linker sequences (SEQ ID NOs: 124-127); FIG. 22 Group IX provides sequences with N-terminal segment cysteine mutations with leading and following linker sequences (SEQ ID NOs: 128-135); FIG. 22 Group X provides sequences with C-terminal segment cysteine mutations with leading and following linker sequences (SEQ ID NOs: 136-138); FIG. 22 Group XI provides sequences with N-terminal segment mutation positions and/or C-terminal segment mutation positions underlined and bolded with leading and following linker sequences as well as secondary alpha helix forming sequence (SEQ ID NOs: 75 and 76-78, 139, 21-23, 64-68); Group FIG. 22 XII provides sequences with N-terminal segment cysteine mutations underlined and bolded with leading and following linker sequences as well as secondary alpha helix forming sequence (SEQ ID NOs: 140-152); FIG. 22 Group XIII provides sequences with C-terminal segment cysteine mutations to form a stapled cTRP underlined and bolded with leading and following linker sequences as well as secondary alpha helix forming sequence (SEQ ID NOs: 153-162); single chain cTRP with 24 repeats.

### DETAILED DESCRIPTION

Epstein Barr virus (EBV) is an orally transmitted gamma herpesvirus that infects B cells and epithelial cells. Most primary infections are asymptomatic, however, EBV is a causative agent of infectious mononucleosis (IM) in children and young adults. Following primary infection, infected individuals become lifelong carriers of EBV, which can lie dormant (i.e., latent) in cells. However, in certain individuals the latent virus can begin to express genes that alter cellular replication, leading to cancer or cancer-like diseases. EBV is also associated with nasopharyngeal carcinoma and lymphoproliferative disorders in immunocompromised patients such as those with HIV/AIDS or in patients undergoing immune suppression for organ transplantation. Thus, vaccines that treat, reduce, or prevent EBV infection of cells or treat EBV-associated diseases would be a major benefit to public health.

A primary goal of most vaccine design strategies is to elicit production of neutralizing antibodies, which are a type of antibody that can inhibit the biological function of its target. Neutralizing antibodies against viruses such as EBV typically function by blocking a virus from entering a cell.

Many previous efforts to design EBV vaccines have included vaccines that target the EBV protein gp350, which is involved in EBV attachment to cells. However, while a phase 2 clinical trial showed that the vaccine could reduce the incidence of IM, it did not protect from EBV infection. Furthermore, EBV vaccine research to-date has not led to the development of an effective human neutralizing antibody against EBV that is widely and clinically available.

In particular embodiments the current disclosure provides EBV vaccines. The EBV vaccine antigens are linked to a multimerization domain, for example presented on a circular tandem repeat protein (cTRP) scaffold or linked to a C4b multimerization domain. A “multimerization domain” is a domain that causes two or more proteins (monomers) to interact with each other through covalent and/or non-covalent association(s). Multimerization domains present in proteins can result in protein interactions that form dimers, trimers, tetramers, pentamers, hexamers, heptamers, etc., depending on the number of units/monomers incorporated into the multimer.

In certain examples cTRPs include 24 repeats of a computationally designed alpha-helical sequence that self-assembles into a toroidal structure utilized to present/scaffold proteins with a selected valency. cTRPs with 12 repeats form toroidal dimers, cTRPs with 8 repeats form toroidal trimers, cTRPs with 6 repeats form toroidal tetramers, and cTRPs with 4 repeats form toroidal hexamer, allowing for the presentation/scaffolding of dimeric, trimeric, tetrameric, hexameric, etc., vaccine antigens.

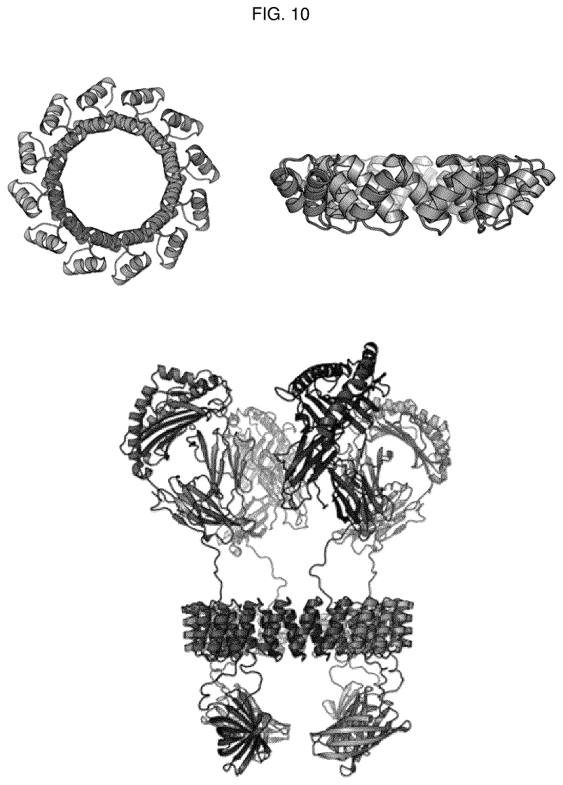

Particular embodiments include tetrameric presentation of the EBV gH/gL heterodimer and trimeric coordination of the EBV gB glycoprotein. Particular embodiments include heptameric presentation of the EBV gH/gL heterodimer. Tetrameric presentation of viral glycoproteins with cTRP scaffolds and heptameric presentation of viral glycoproteins with C4b multimerization domains serve to improve immunogenicity, expression, and/or stability of vaccine antigens. Trimeric presentation can afford the same advantages as well as providing the unique ability over other scaffolds to coordinate/stabilize herpesvirus gB trimer pre-fusion states given the serendipitous alignment of predicted fusion attachment points between the fusion proteins and the scaffold (see, FIG. 10 (50 A)). Presentation of gB in a prefusion state supports the elicitation of strong neutralizing antibody responses.

The designs also may be used as immunogens for subsequent isolation of binding/neutralizing antibodies against the presented glycoproteins with therapeutic/diagnostic/research applications.

EBV vaccines disclosed herein can be formulated to treat an EBV-infected subject or a subject at risk of EBV infection. In particular embodiments, EBV vaccines reduce the risk or severity of EBV infection, and/or induce an immune response against EBV. Treating EBV can reduce EBV infection and/or treat a condition associated with EBV infection, such as infectious mononucleosis or lymphoproliferative disorder. In particular embodiments, use of EBV vaccines disclosed herein lower the risk for nasopharyngeal carcinoma in a subject.

Aspects of the disclosure are now described with additional detail and options as follows: (i) EBV Vaccine Antigens; (ii) cTRP Scaffolds; (iii) Additional Multimerization Domains; (iv) Additional Components; (v) Recombinant Production; (vi) Compositions for Administration; (vii) Methods of Use; (viii) Kits; (ix) Exemplary Embodiments; (x) Experimental Examples; and (xi) Closing Paragraphs. These headings are provided for organization purposes only and do not limit the scope or interpretation of the disclosure.

(i) EBV Vaccine Antigens. EBV vaccines disclosed herein can include an EBV antigen. In particular embodiments, the EBV vaccines include an EBV antigen that includes a gH/gL complex, and/or a D-I/D-II groove and the DI/DII linker helix of a gH/gL complex.

In particular embodiments, the EBV vaccine can include a subunit vaccine. A subunit vaccine can refer to a vaccine that does not contain a whole live or killed pathogen, but only a subunit (e.g., a single protein or protein fragment) of the pathogen that stimulates an immune response against the pathogen. In particular embodiments, the EBV subunits vaccines can include vaccine proteins including (i) gH/gL or a fragment of gH/gL; and/or (ii) gB or a fragment of gB.

Particular examples of vaccine antigens include the EBV envelope glycoprotein H (gH), the EBV envelope glycoprotein L (gL), the EBV envelope glycoprotein B (gB), or the EBV glycoprotein 42 (gp42). Particular examples of vaccine antigens include fragments of gH, gL, gB, or gB42. In particular embodiments, the vaccine antigens include fragments of gB.

In certain examples, the EBV vaccine antigen has the sequence as set forth in SEQ ID NOs: 1, 2, 3, 4, 5, 281, 282, 286, 287, 288, or 289 or has a sequence with at least 90% sequence identity to the sequence as set forth in SEQ ID NOs: 1, 2, 3, 4, 5, 281, 282, 286, 287, 288, or 289. Combinations of the EBV vaccine antigens can also be used. In preferred embodiments, the EBV vaccine antigen includes SEQ ID NOs: 286 or 288.

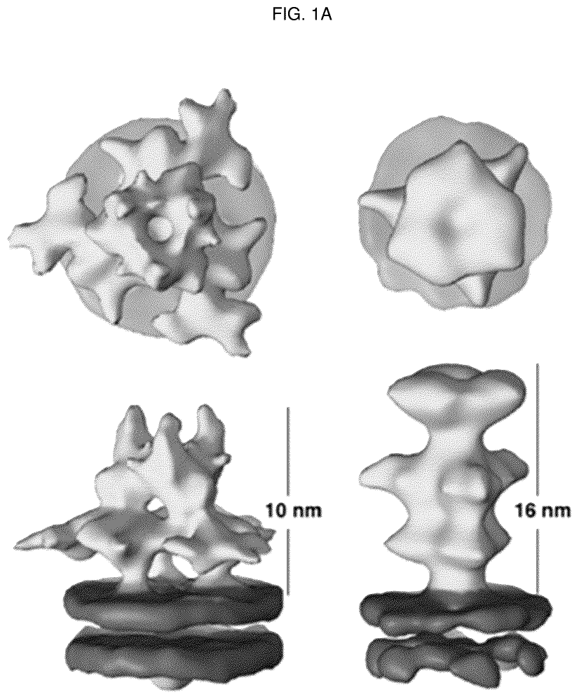

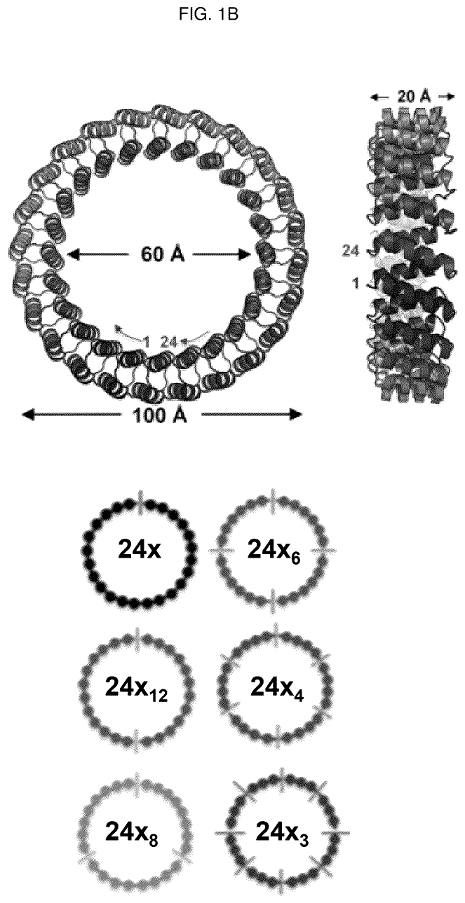

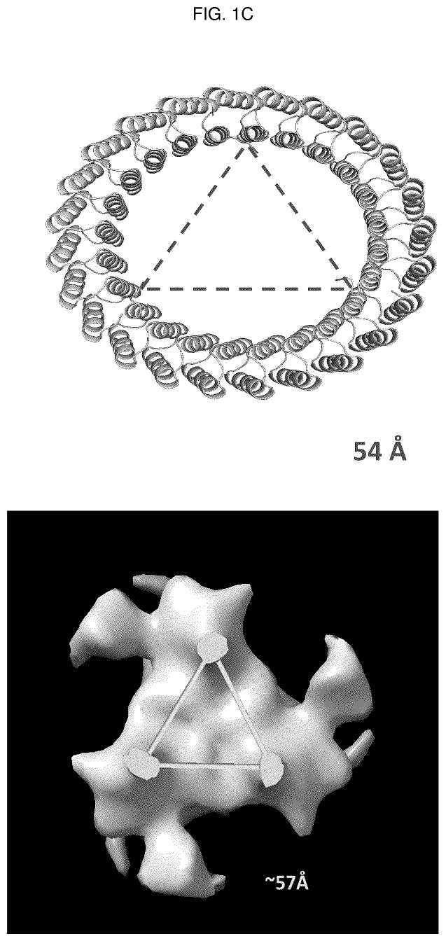

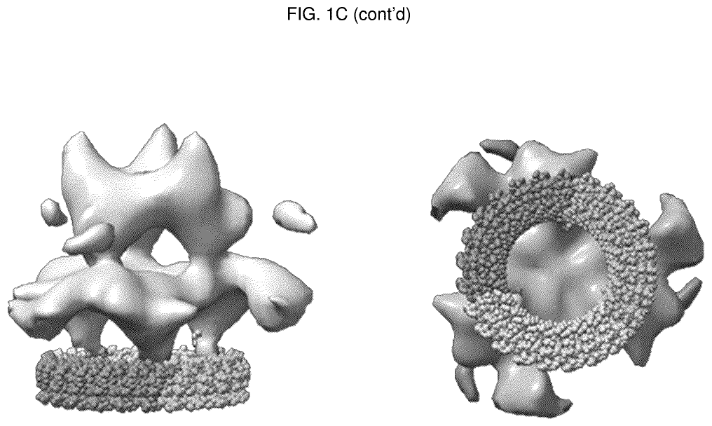

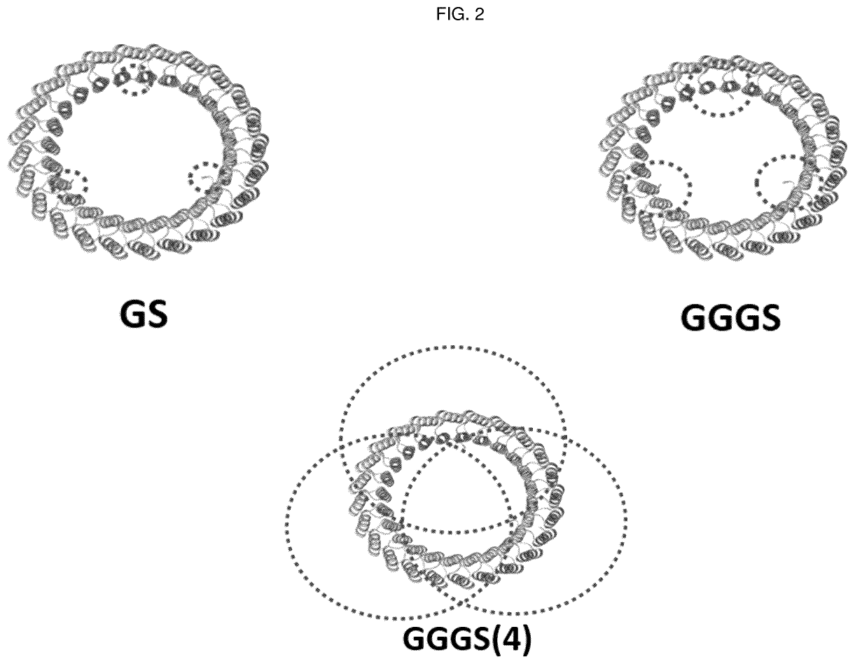

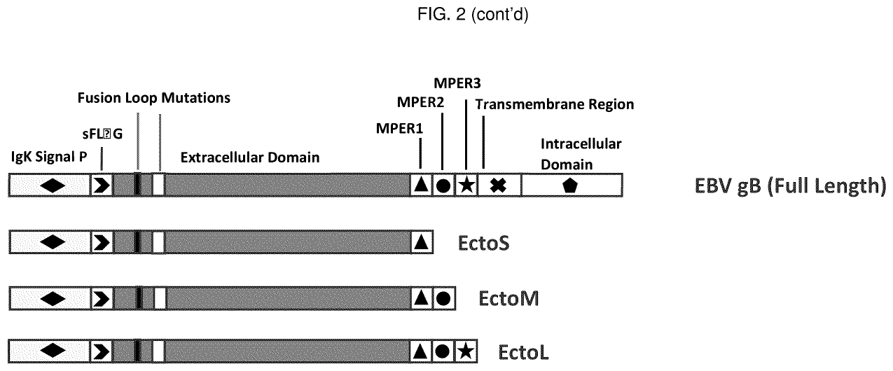

(ii) cTRP Scaffolds. As indicated, EBV vaccine antigens can be presented in multimerized form on a cTRP scaffold. cTRPs include repetitive α-helical structures joined by linkers. Referring to, for example, FIGS. 1B, 1C, and 2, each repetitive α-helical structure includes an outer α-helix and an inner α-helix. In particular embodiments, an α-helical structure is repetitive (e.g., structurally repetitive) when following (i) stacking with an adjacent α-helical structure; and (ii) comparison using root-mean-square-deviation (RMSD), the distance between corresponding atoms of the stacked outer α-helices and the stacked inner α-helices is within 2 angstrom (Å); within 1.5 Å; within 1 Å; within 0.5 Å; within 0.4 Å; or within 0.2 Å.

Exemplary amino acid sequences that generate an α-helix include VEELLKLAKAAYYS (SEQ ID NO: 52); VEEAYKLALKL (SEQ ID NO: 53); VEELLKLAEAAYYS (SEQ ID NO: 54); PTEALLKLIAEAK (SEQ ID NO: 206); ETEAKEEAEKALKE (SEQ ID NO: 207); STEAKEEAIKALKE (SEQ ID NO: 208); ELEAKVLAEKALKE (SEQ ID NO: 209); ETEAKLEAEKALKE (SEQ ID NO: 210); PTEVLLELIAEAS (SEQ ID NO: 17); KEEVKEKFLKELSK (SEQ ID NO: 211); KEEVKRKFLKELSK (SEQ ID NO: 212); KAEVKREFLWELSL (SEQ ID NO: 213); KEEVKEKFLAELEK (SEQ ID NO: 214); REEVKEKFLKELRK (SEQ ID NO: 18); KEEVKEKFLKELSF (SEQ ID NO: 19); KEEVKKKFWKELSL (SEQ ID NO: 20); KREVKRWFLFELRK (SEQ ID NO: 215); KAEVKLKFLFELSF (SEQ ID NO: 216); KEEVKEKFLKELFK (SEQ ID NO: 217); TTEALLILIAEAS (SEQ ID NO: 218); VEQQKQRFKELVKK (SEQ ID NO: 219); TAIAQILAIKASAK (SEQ ID NO: 25); TELERALRYAKKV (SEQ ID NO: 220); TELERALRYAVKV (SEQ ID NO: 26); TELEQALRYAKFV (SEQ ID NO: 27); LELTRALAYAKKV (SEQ ID NO: 28); TELERALRYAKLV (SEQ ID NO: 29); TELERALRYAKYV (SEQ ID NO: 30); PELEYALAYAKKV (SEQ ID NO: 31); TELERALIFAEAV (SEQ ID NO: 221); TELDRALWYAKKV (SEQ ID NO: 222); TELERALLYAKKV (SEQ ID NO: 223); TELERALAYARLV (SEQ ID NO: 224); TELERALRYAEKV (SEQ ID NO: 225); TELERALWYAKKV (SEQ ID NO: 226); SAIATAYIALAEYL (SEQ ID NO: 227); EALLKAIEIAIKL (SEQ ID NO: 228); SAIAEAYIALARYL (SEQ ID NO: 229); SALAQILAIYASAY (SEQ ID NO: 230); TLFLRALKLAKEV (SEQ ID NO: 231); ELYIRVLAIVAEAE (SEQ ID NO: 232); TKLELALKLALKK (SEQ ID NO: 233); KLYIEVLAIVAEAE (SEQ ID NO: 234); ELYIRVLAIVAKAE (SEQ ID NO: 235); KLYIEVLAIVAKAE (SEQ ID NO: 236); LEQALKILKVAAEL (SEQ ID NO: 39); VEEAVKRALKLKTKL (SEQ ID NO: 40); LEQALKILEVAAEL (SEQ ID NO: 41); LEQALKILEVAAKL (SEQ ID NO: 42); VEEAVKRAMKLKTKL (SEQ ID NO: 43); as well as SEQ ID NO: 58-63; 73, 74, 80 and 81.

Each repetitive α-helical structure includes 2 sequences that each form an α helix. The two sequences forming a helices within each repetitive structure can be identical or can have at least 99%, at least 98%, at least 97%, at least 96%, at least 95%, at least 94%, at least 93%, at least 92%, at least 91%, at least 90% or at least 85% sequence identity to the other within the structure. Thus, in particular embodiments, a repetitive α-helical structure of a cTRP disclosed herein would include at least two sequences from SEQ ID NOs. 17-20, 25-31, 39-43, 52-54, 206-236, 58-63, 73, 74, 80 and 81 to generate an outer α helix and an inner α helix, respectively.

Many cTRPs have a circular architecture stabilized by interactions between the first and last repeats, obviating the need for capping repeats to maintain solubility and making them more tolerant than open repeat architectures to imperfections in the designed geometry. Thus, in particular embodiments, a circular protein (e.g., a protein having a circular architecture) is one wherein the N-terminal and C-terminal ends of the protein are naturally found within 10 Å following expression and folding. In particular embodiments, a circular protein is one wherein the N-terminal and C-terminal ends of the protein are naturally found within 10 Å; within 9 Å; within 8 Å; within 7 Å; within 6 Å; within 5 Å; within 4 Å; within 3 Å; within 2 Å; within 1 Å; or within 0.5 Å; following expression and folding. Naturally found means that the cTRP is self-folding. In particular embodiments, a circular protein is one designated as such by the teachings of Kajava, A. V. Tandem repeats in proteins: from sequence to structure. J. Struct. Biol. 179, 279-288, doi:10.1016/j.jsb.2011.08.009 (2012).

Many cTRPs are left-handed proteins. In particular embodiments, to compute the handedness of helical bundles formed by cTRPs, an approximate helical bundle axis curve can be generated by joining the location of repeat-unit centers of mass in a sliding fashion along the protein chain. The handedness can then be determined by computing the directionality of the winding of the polypeptide chain about this axis curve. In particular embodiments, a left-handed protein is one wherein the protein is designated as such by the teachings of Kajava, A. V. Tandem repeats in proteins: from sequence to structure. J. Struct. Biol. 179, 279-288, doi:10.1016/j.jsb.2011.08.009 (2012).

In particular embodiments, the left-handedness of particular cTRPs is due in part to the use of inter-helical turns whose geometry naturally imparts a handedness to the resulting helical bundle. The 3-residue ‘GBB’ (αL-β-β) turn type used in particular embodiments prefers a left-handed dihedral twist between the connected helices, while a ‘GB’ turn can result in a right-handed geometry. Both these turn types are also compatible with canonical helix capping interactions.

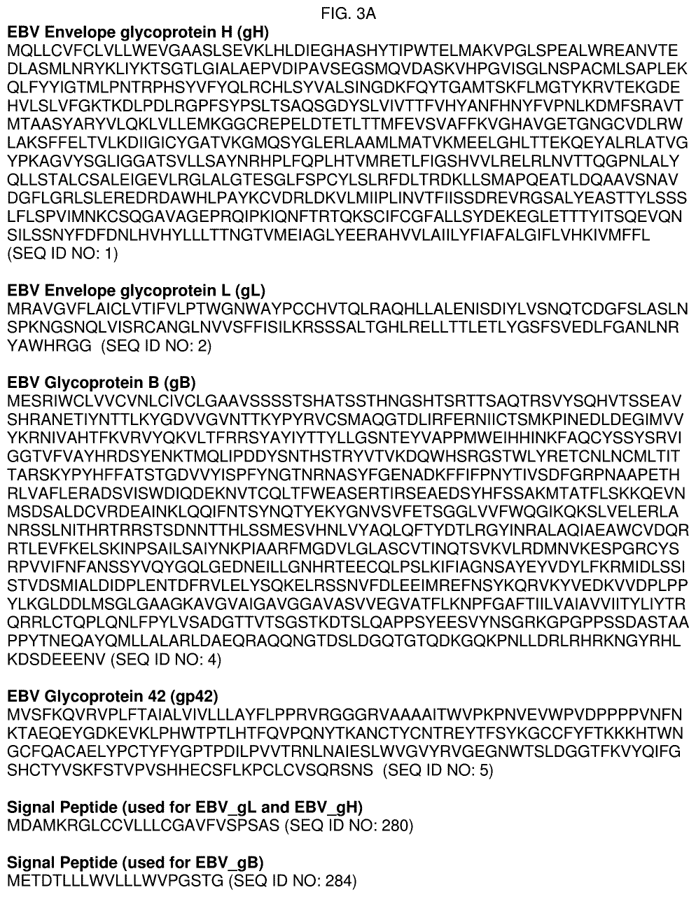

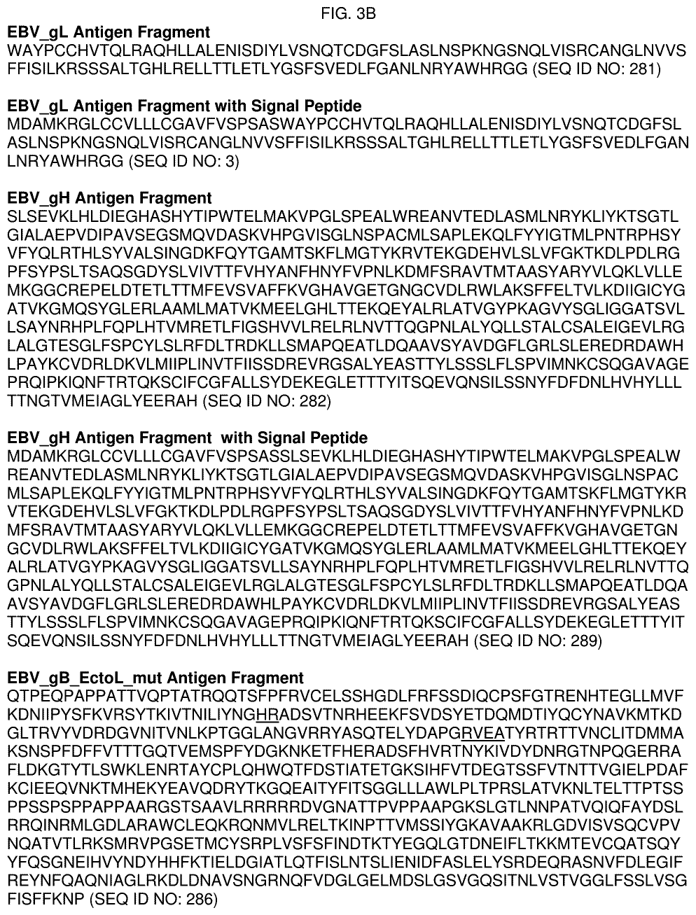

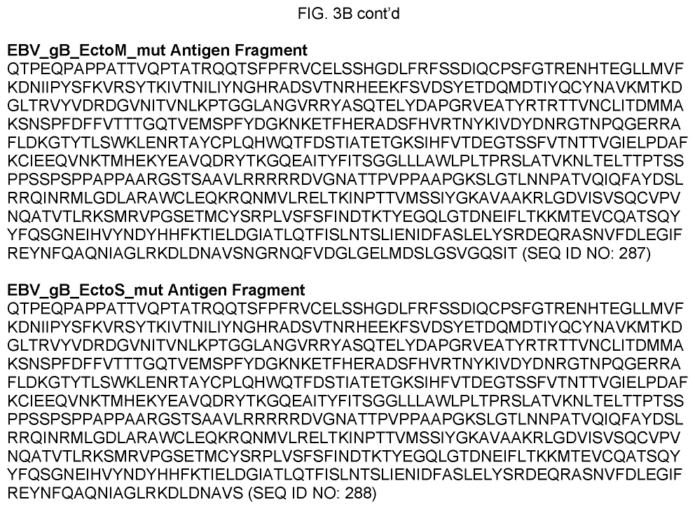

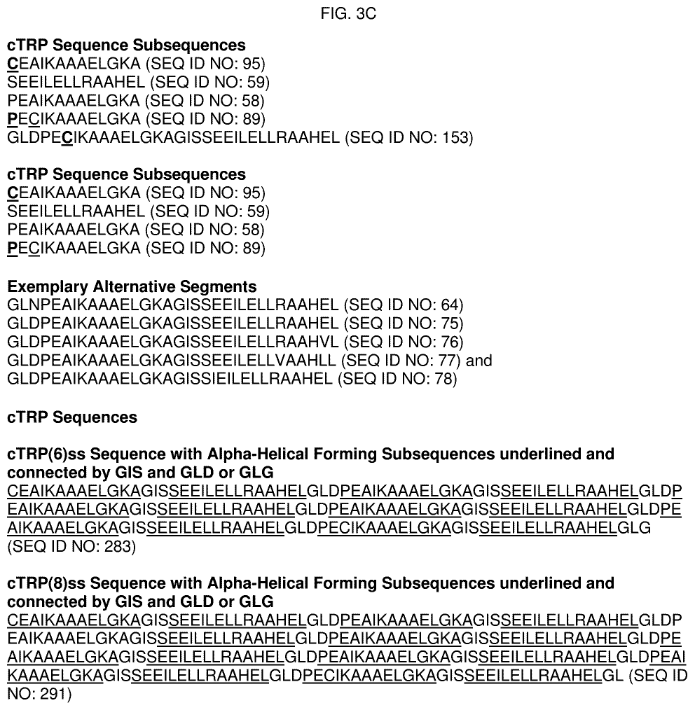

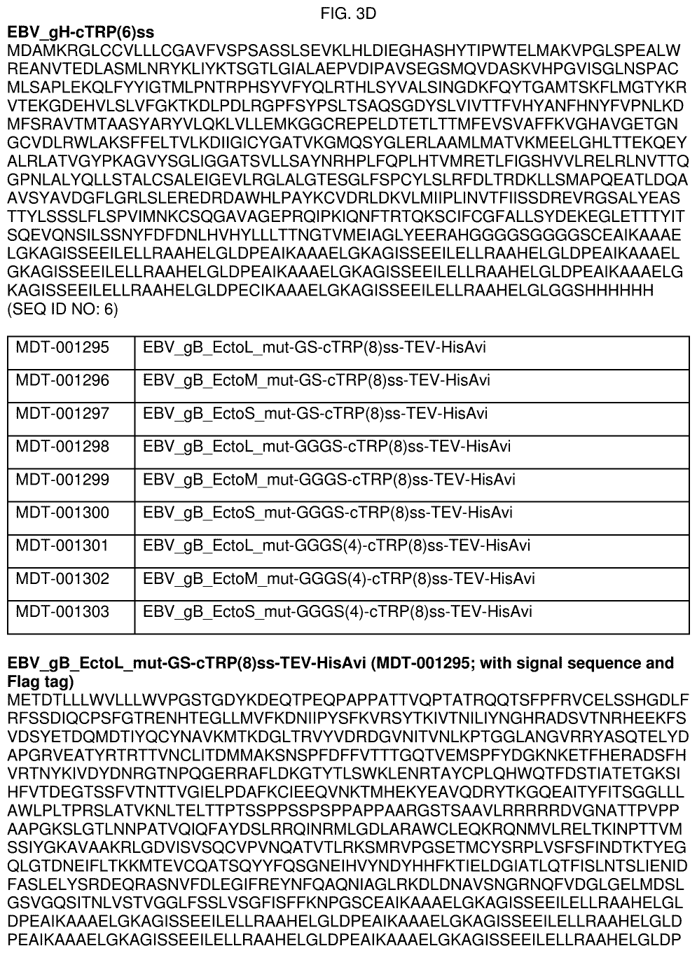

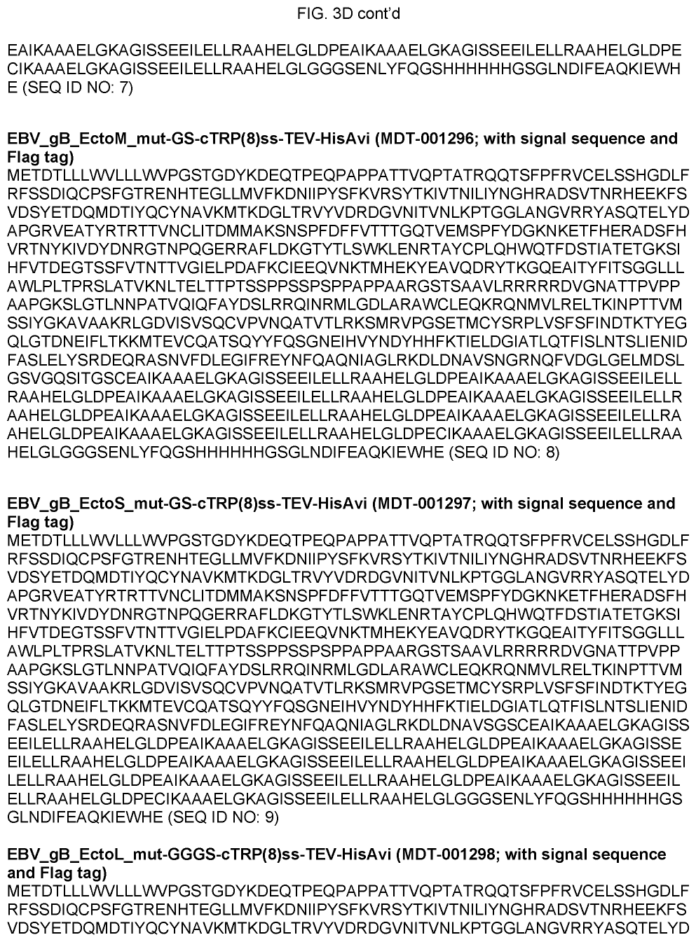

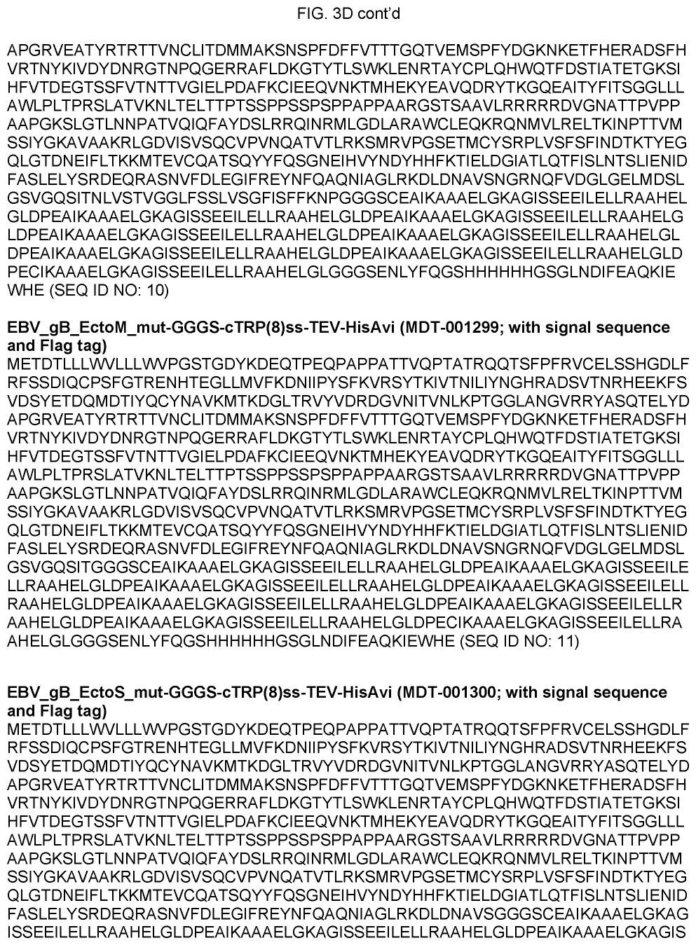

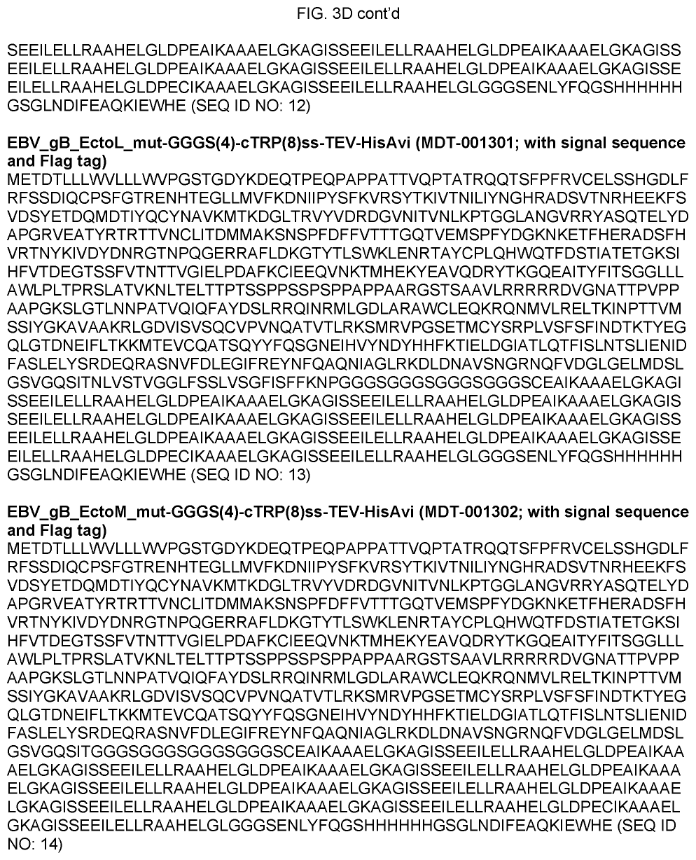

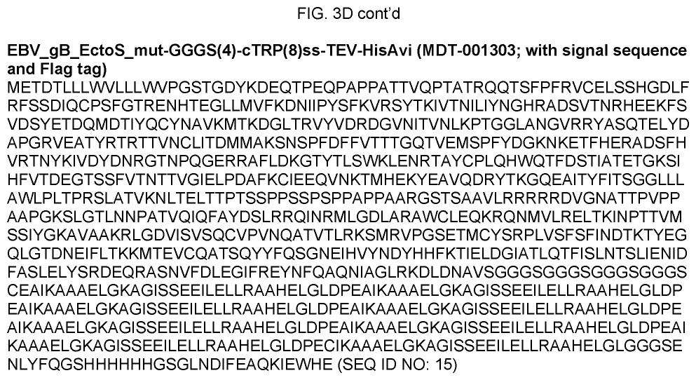

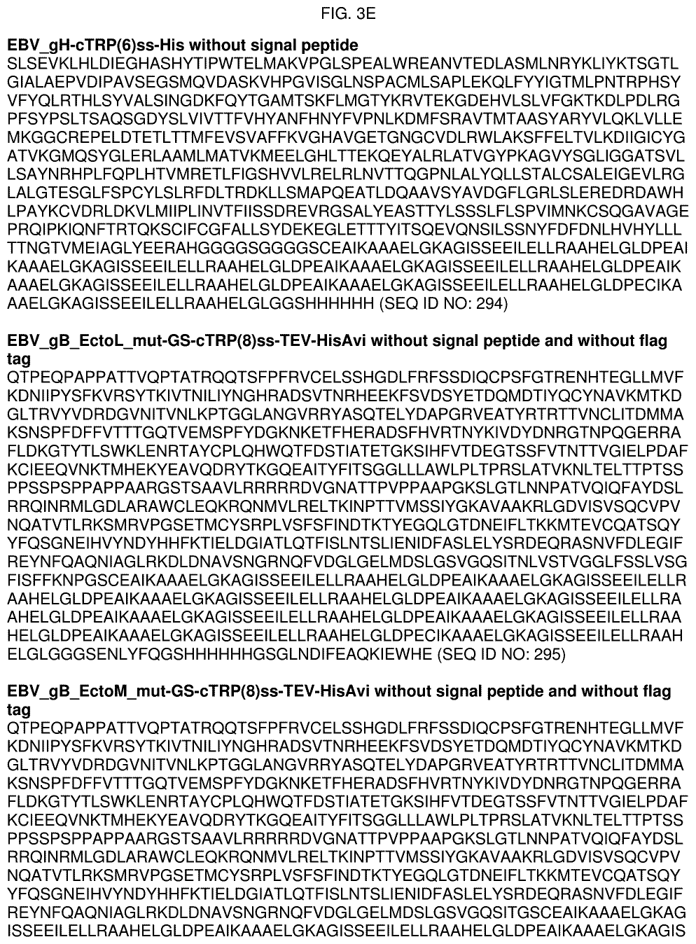

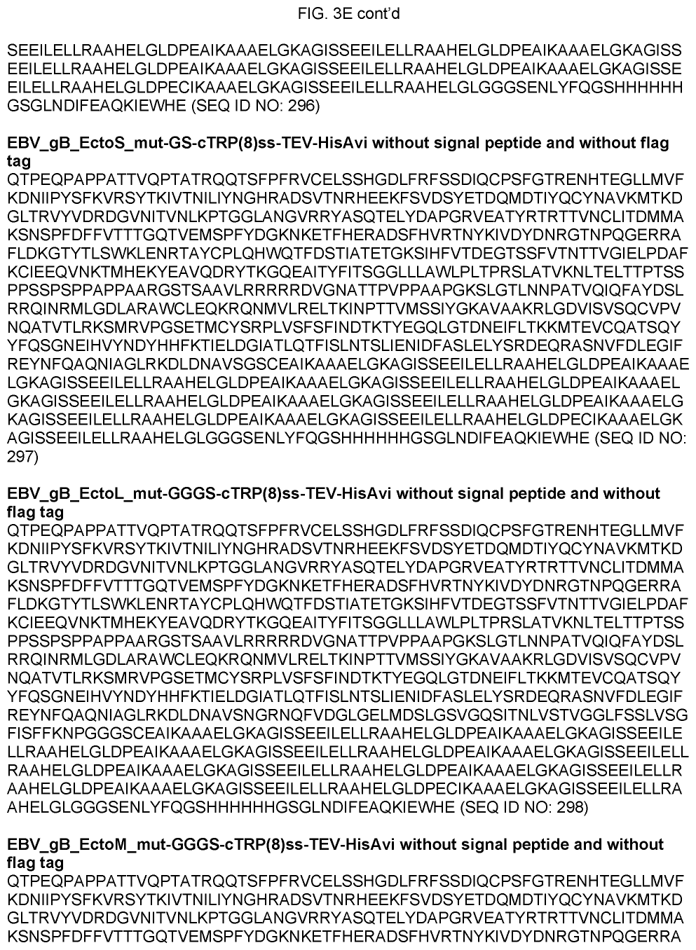

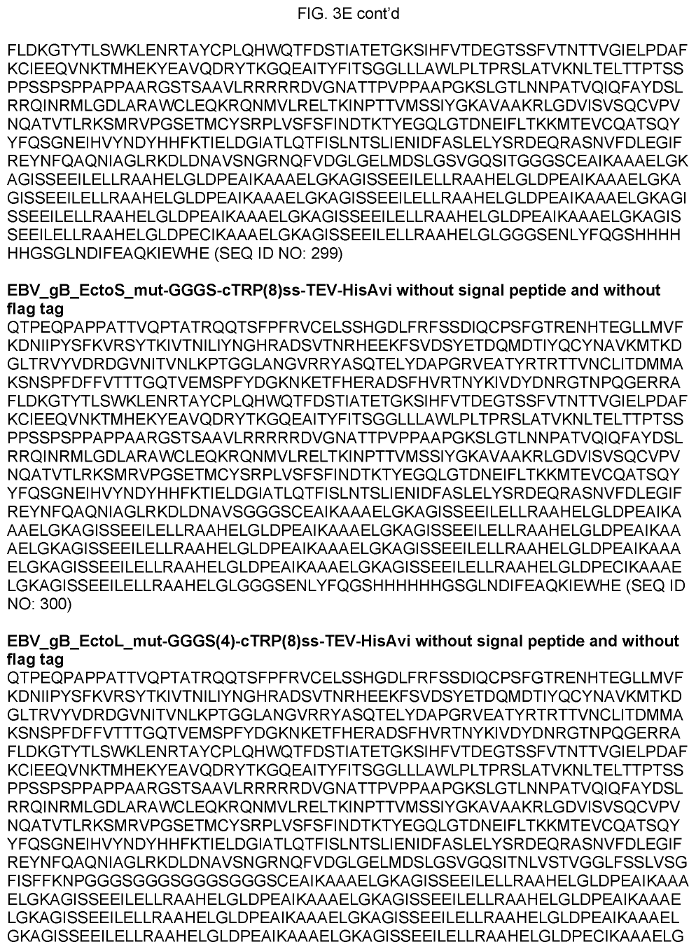

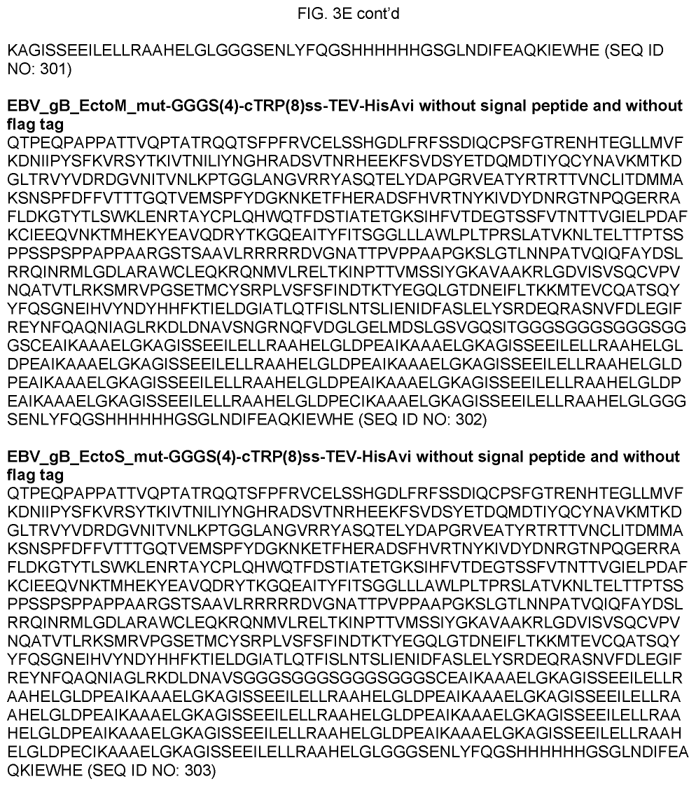

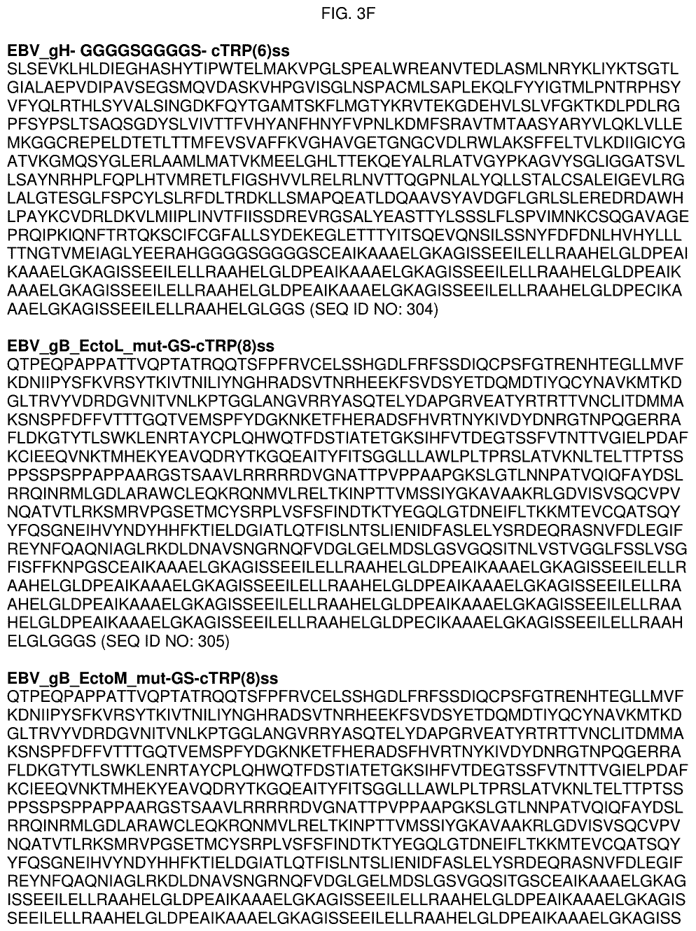

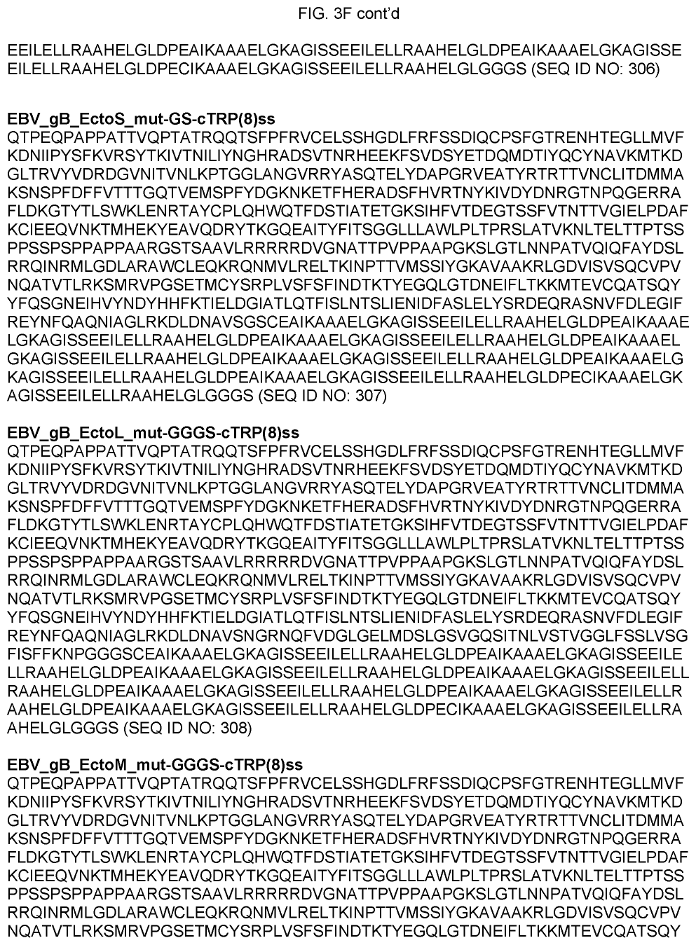

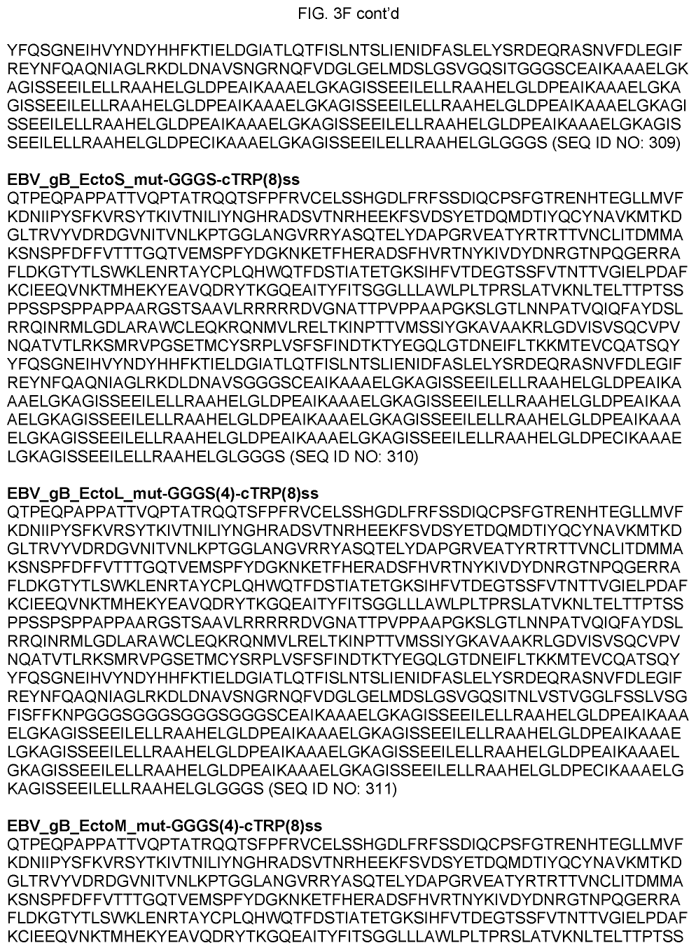

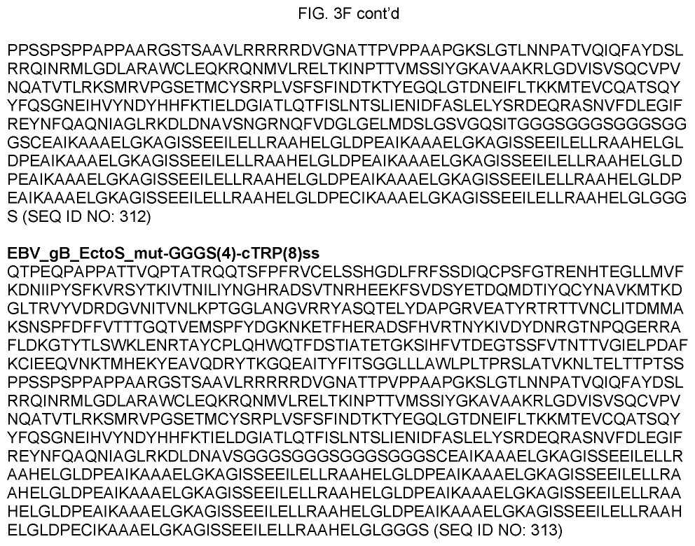

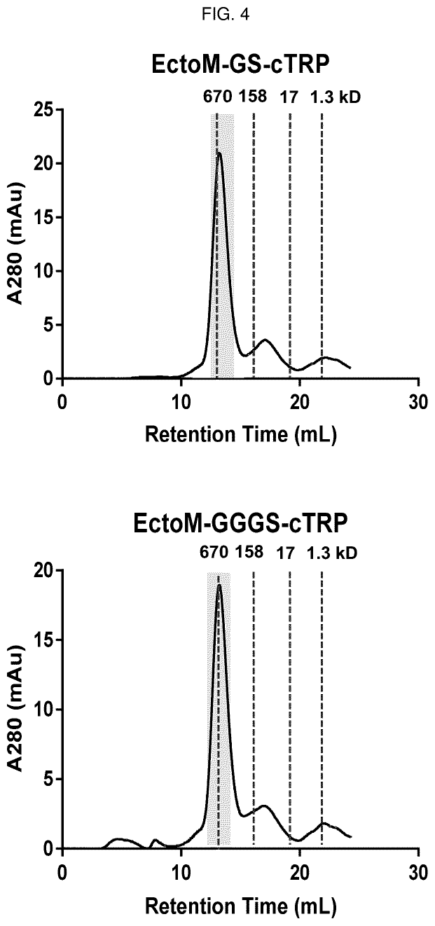

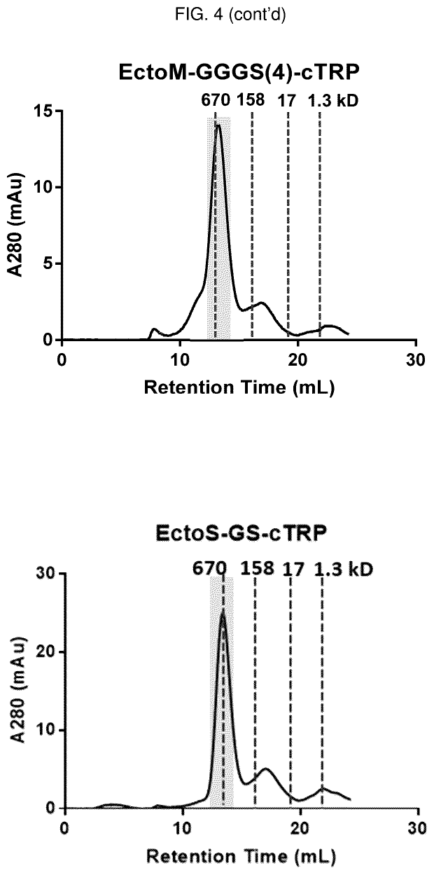

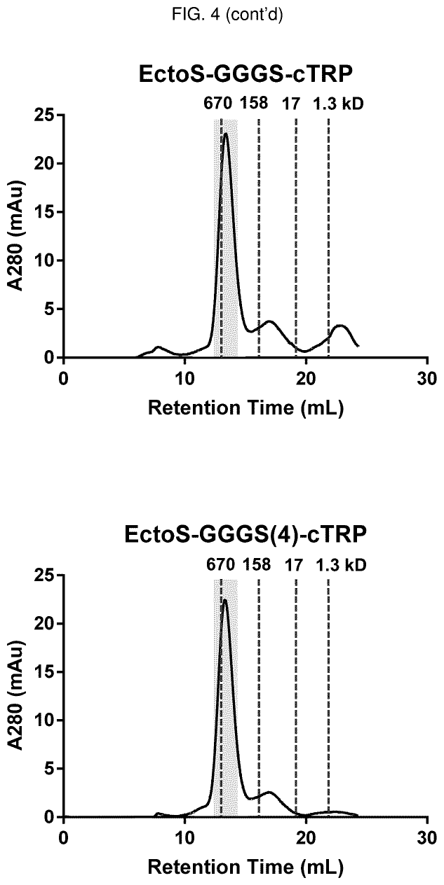

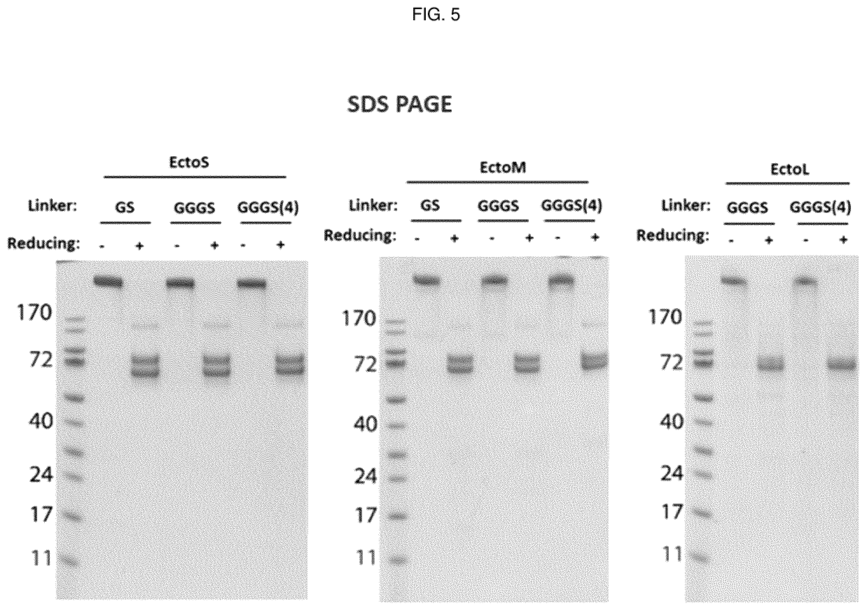

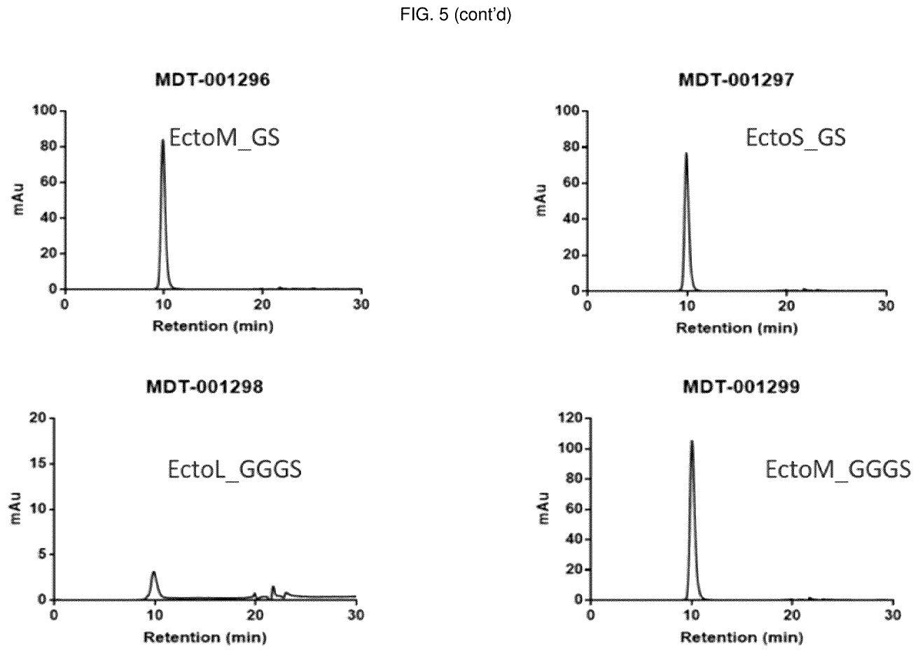

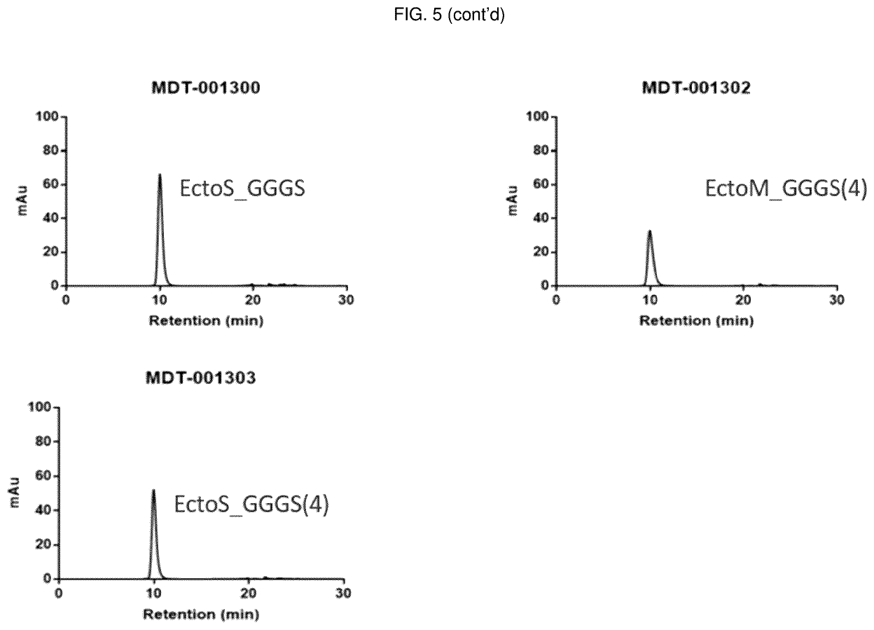

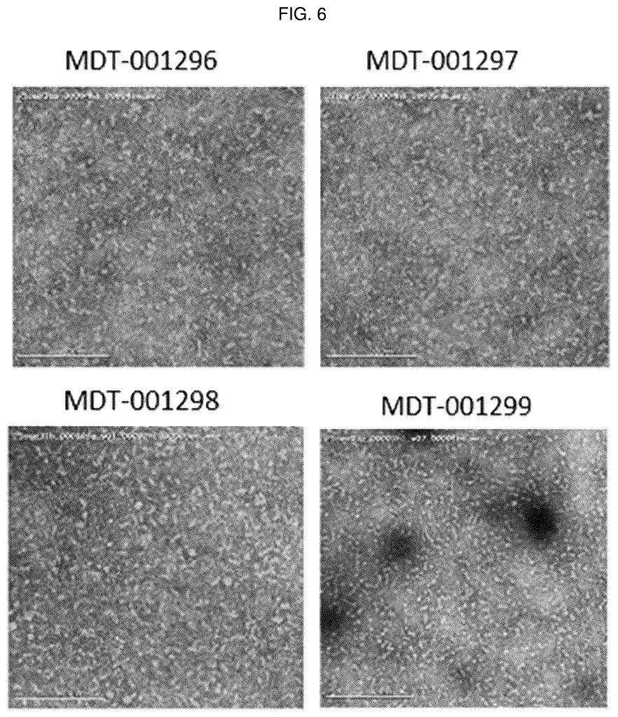

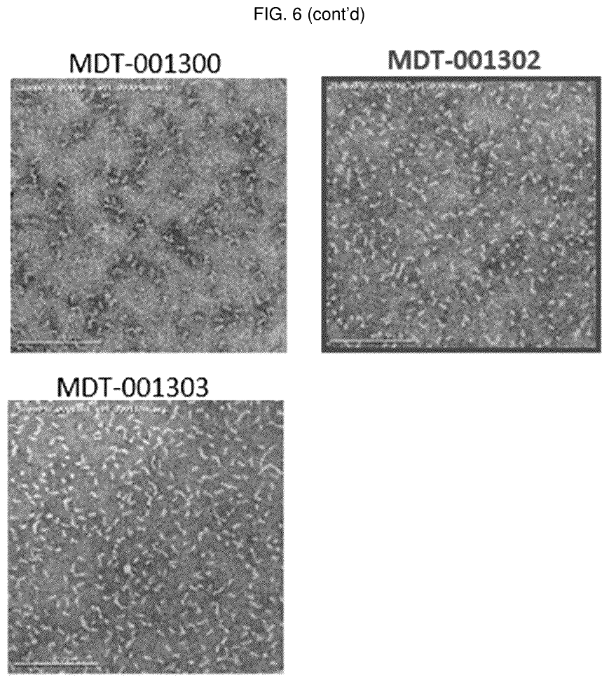

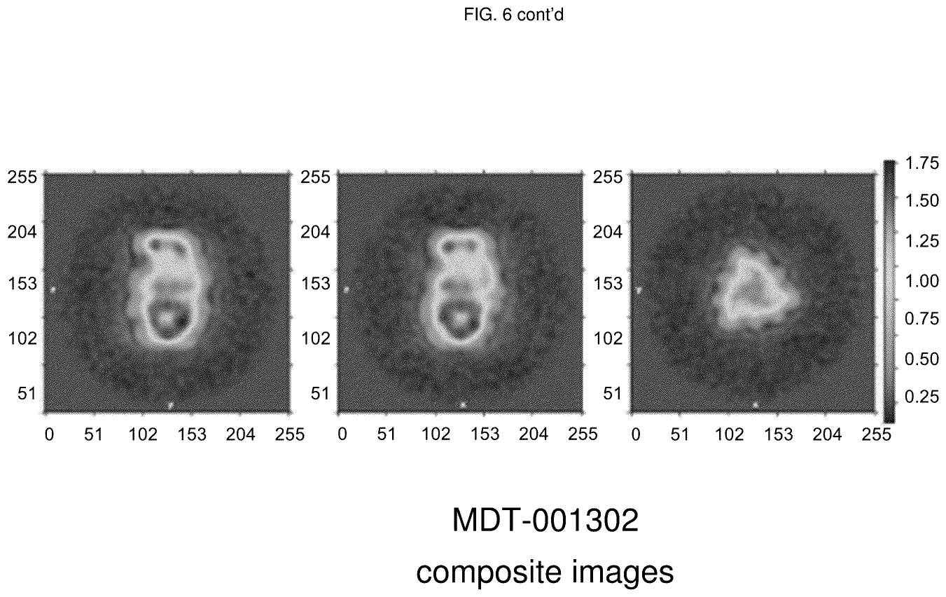

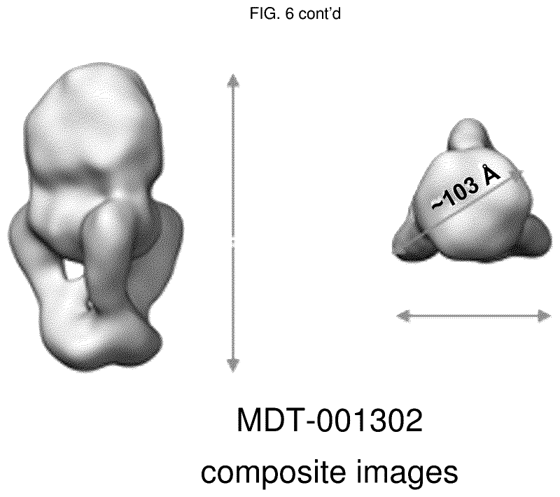

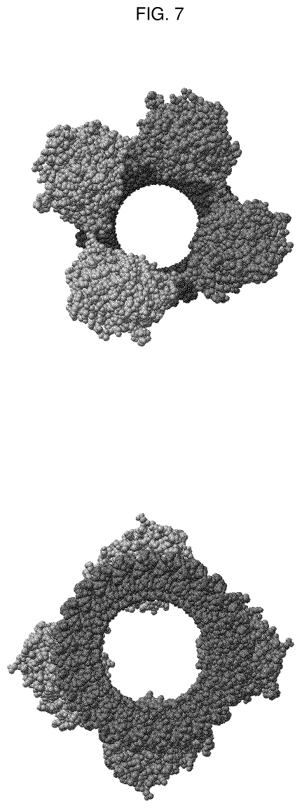

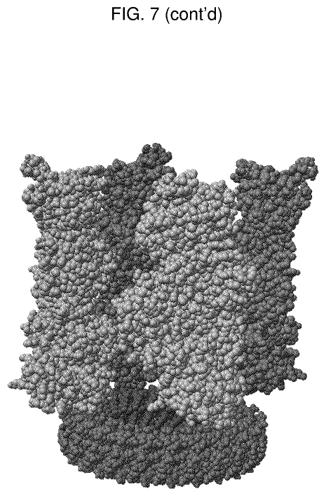

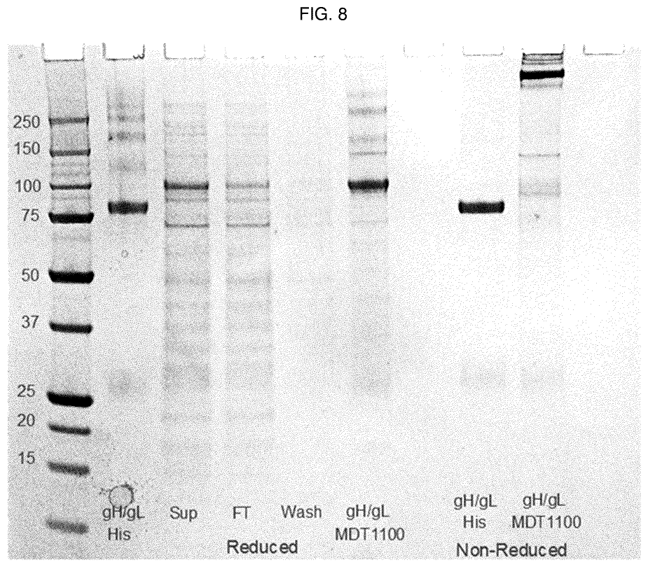

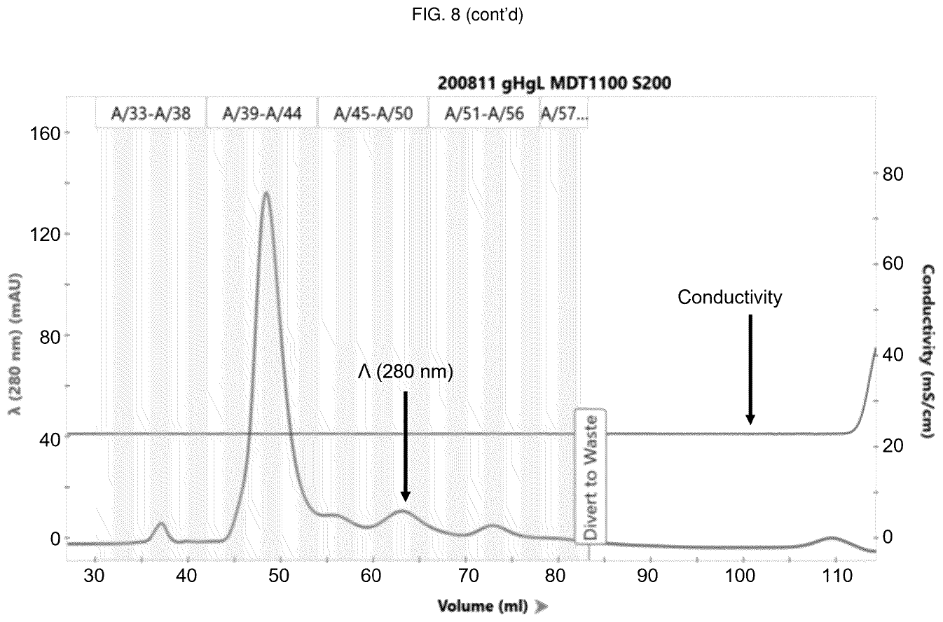

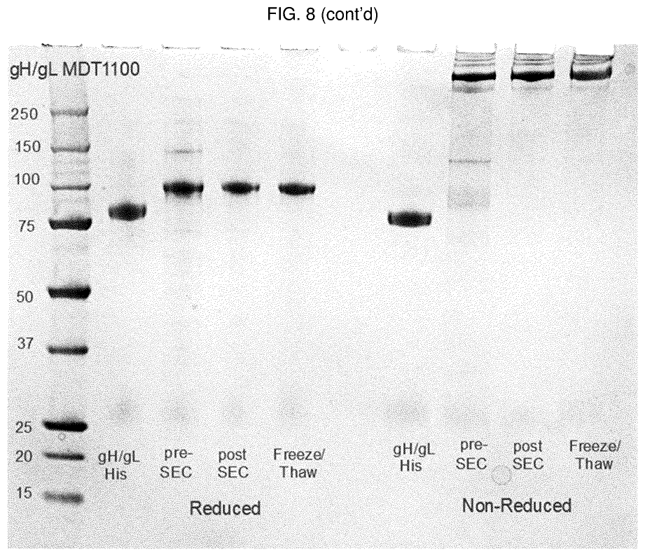

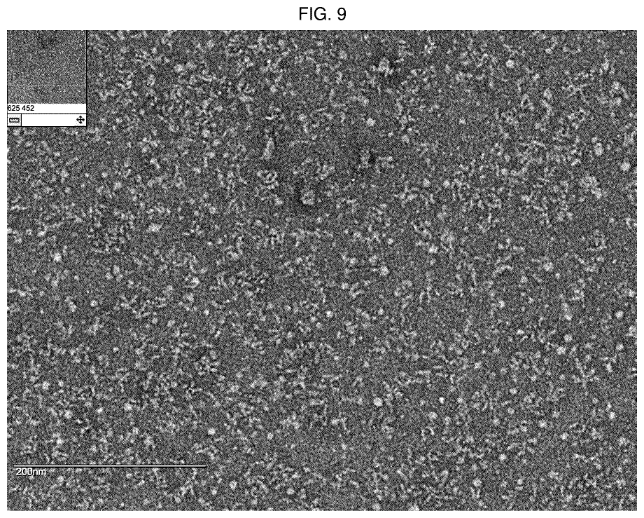

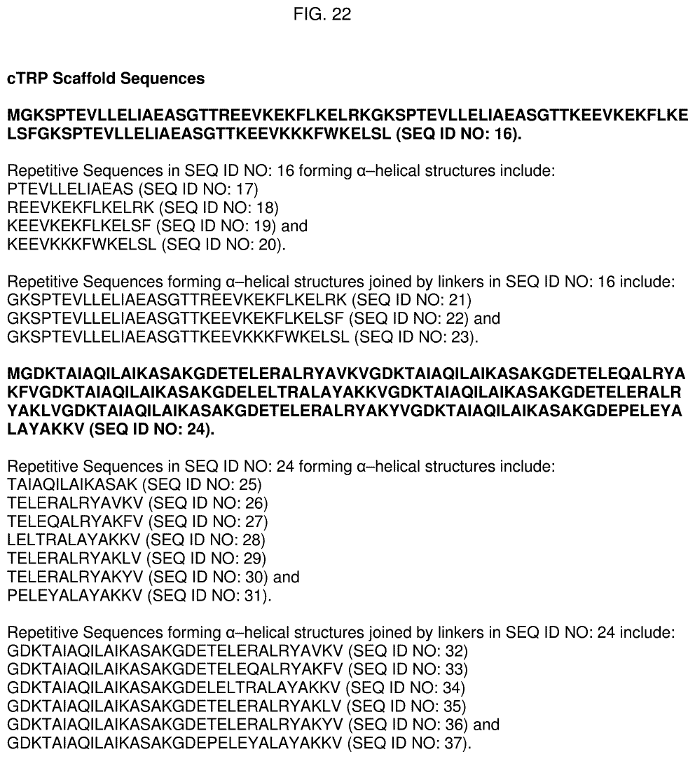

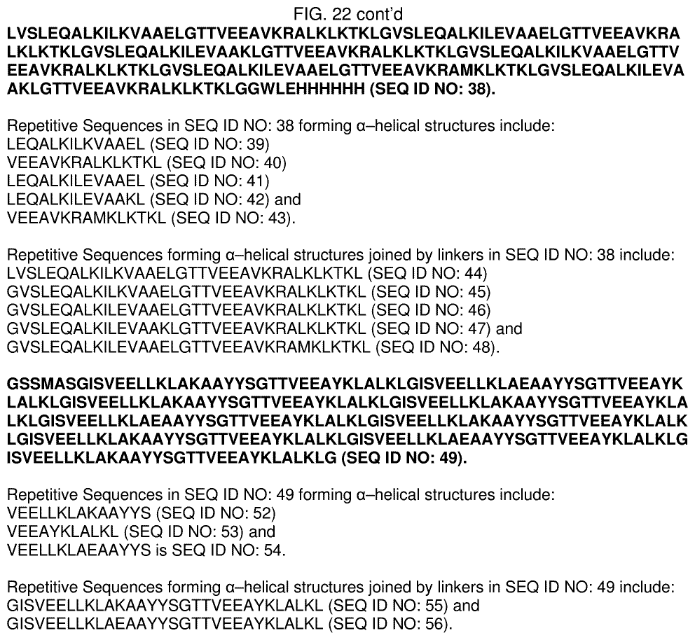

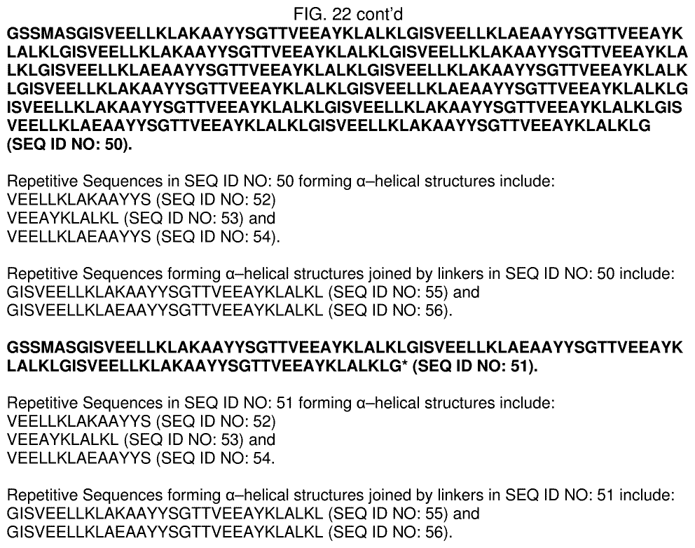

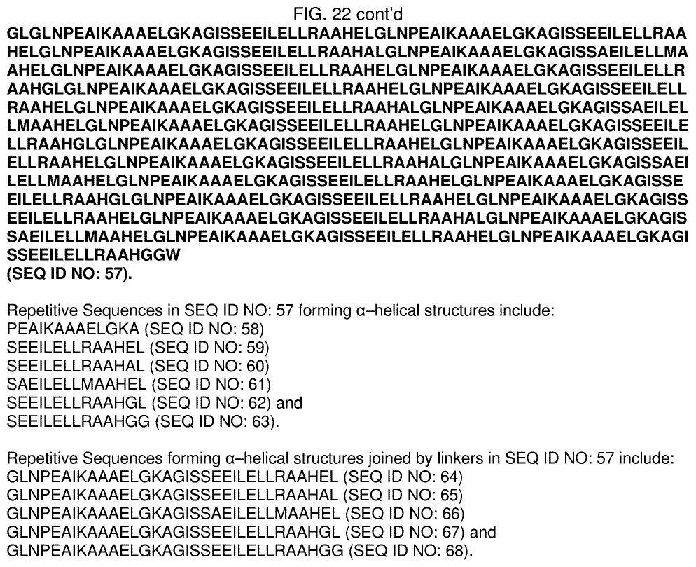

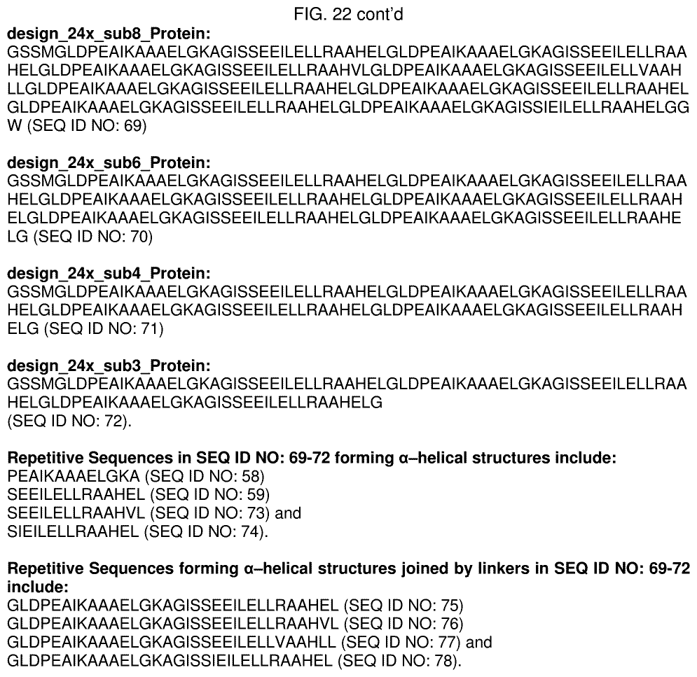

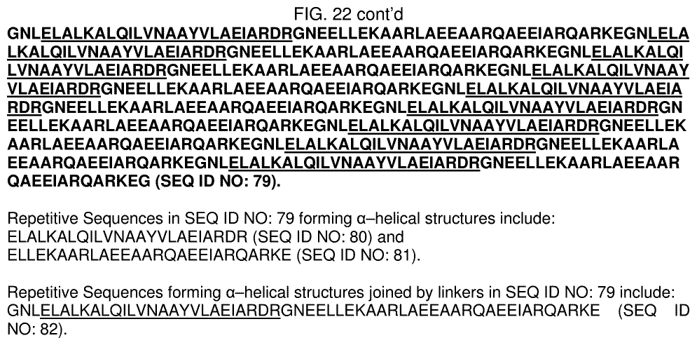

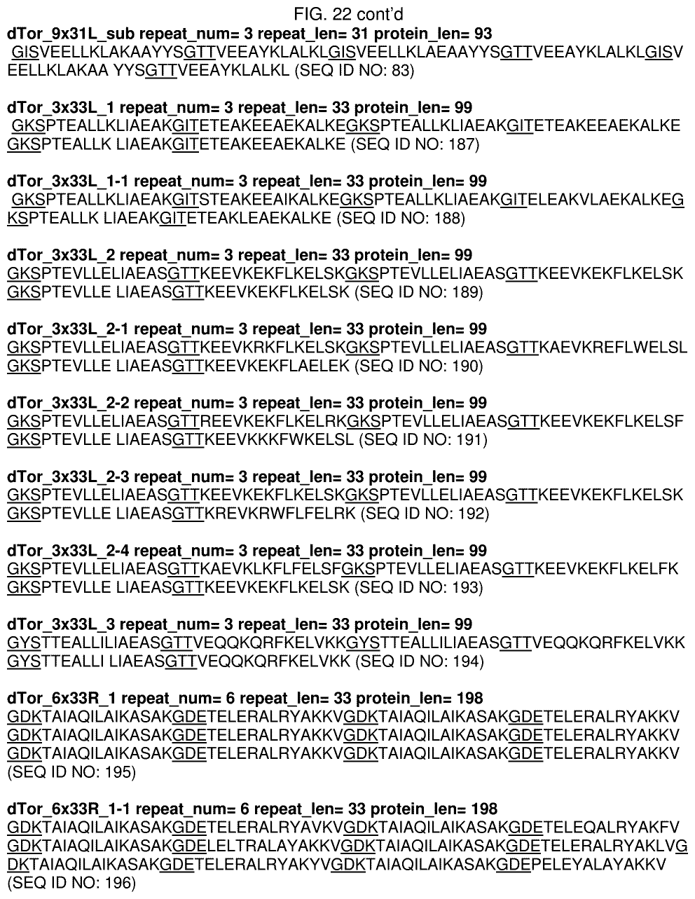

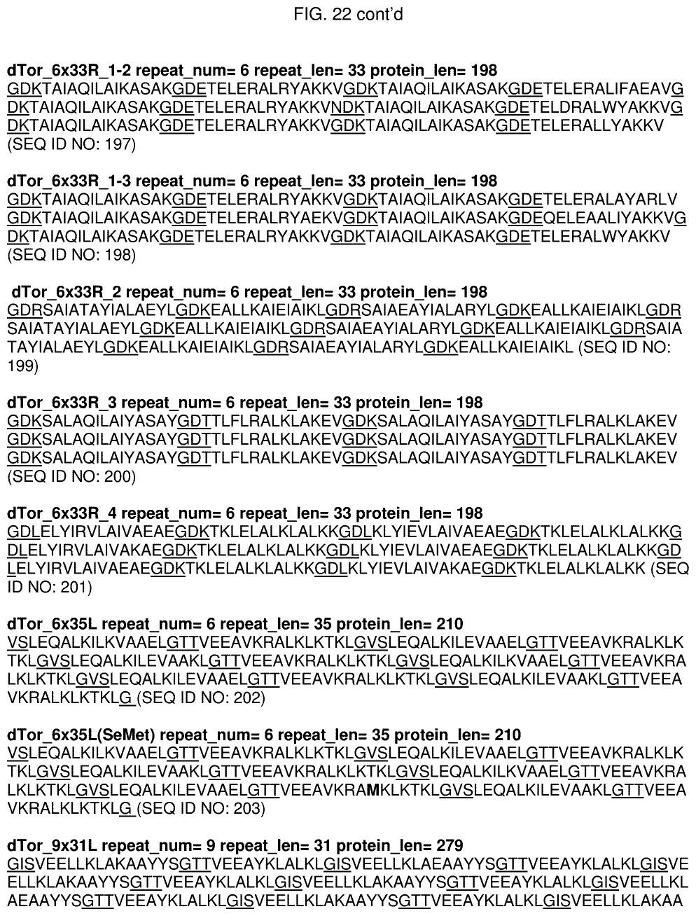

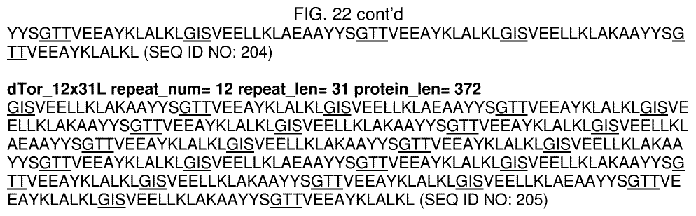

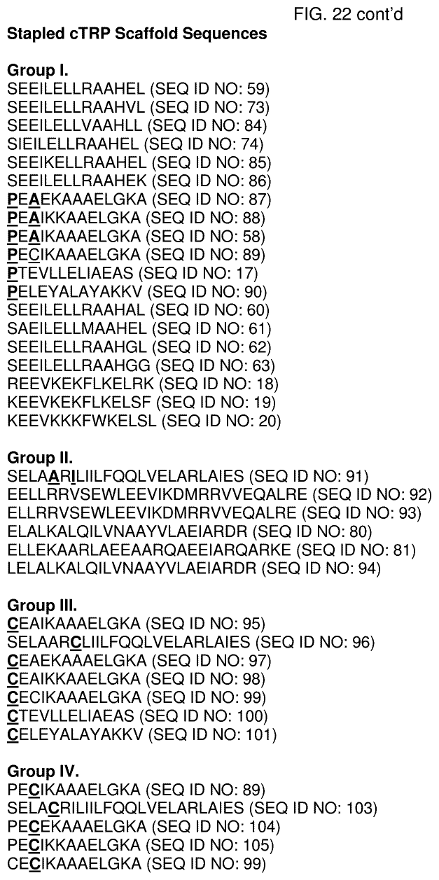

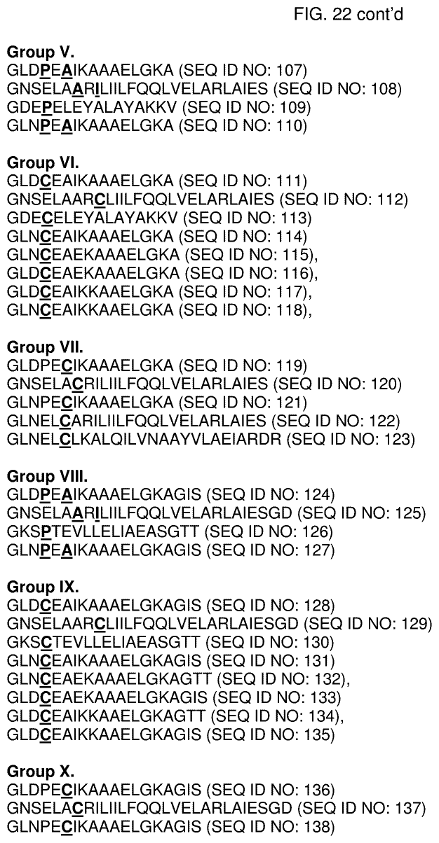

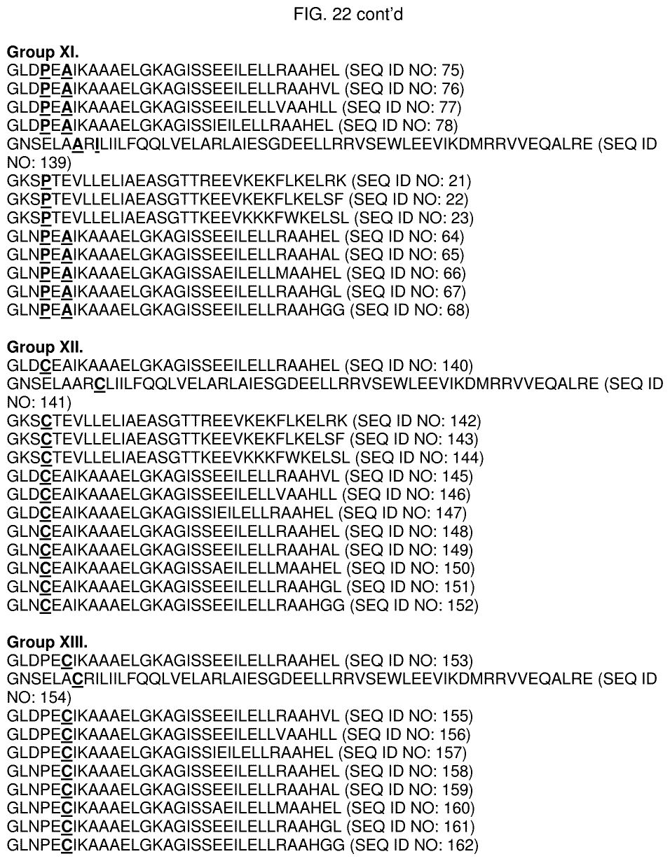

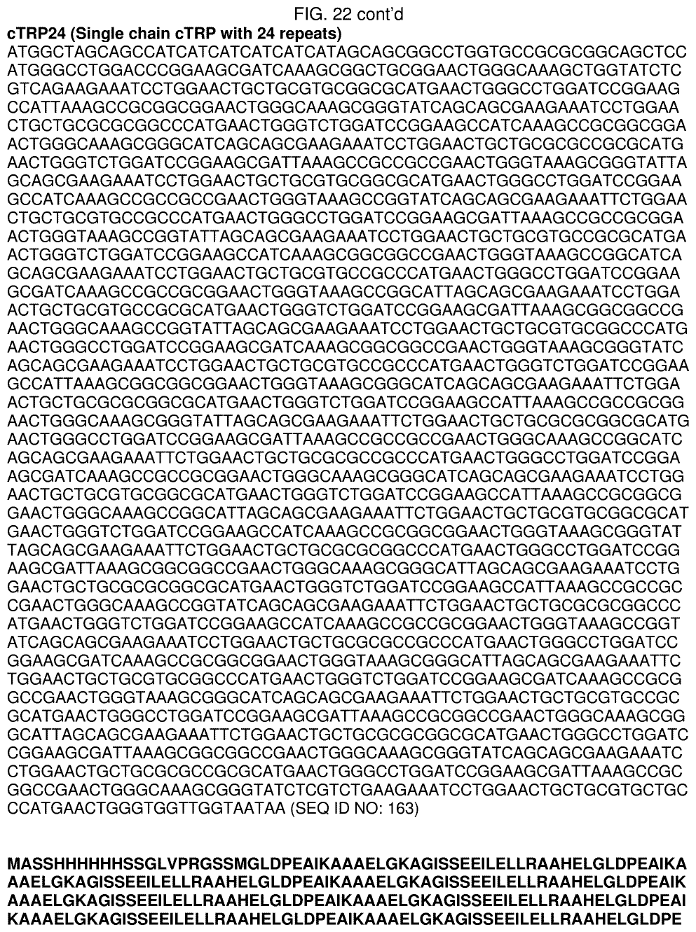

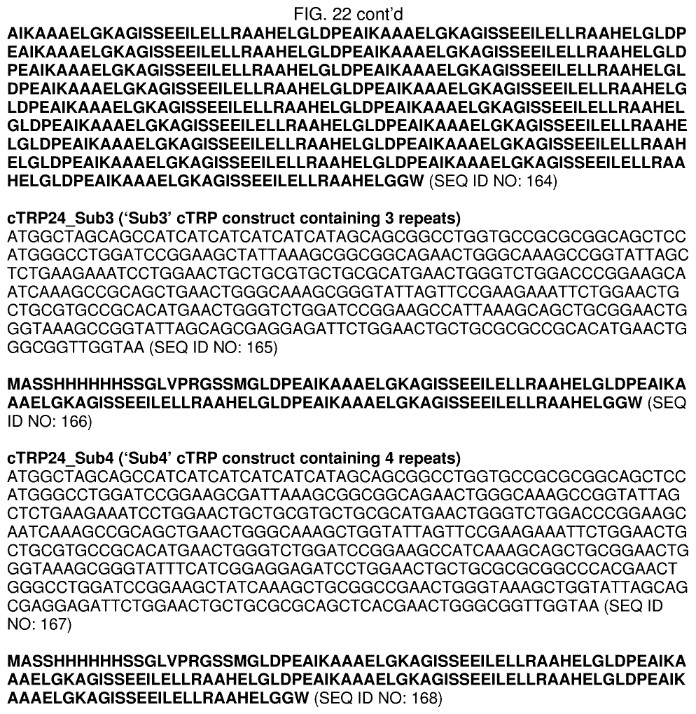

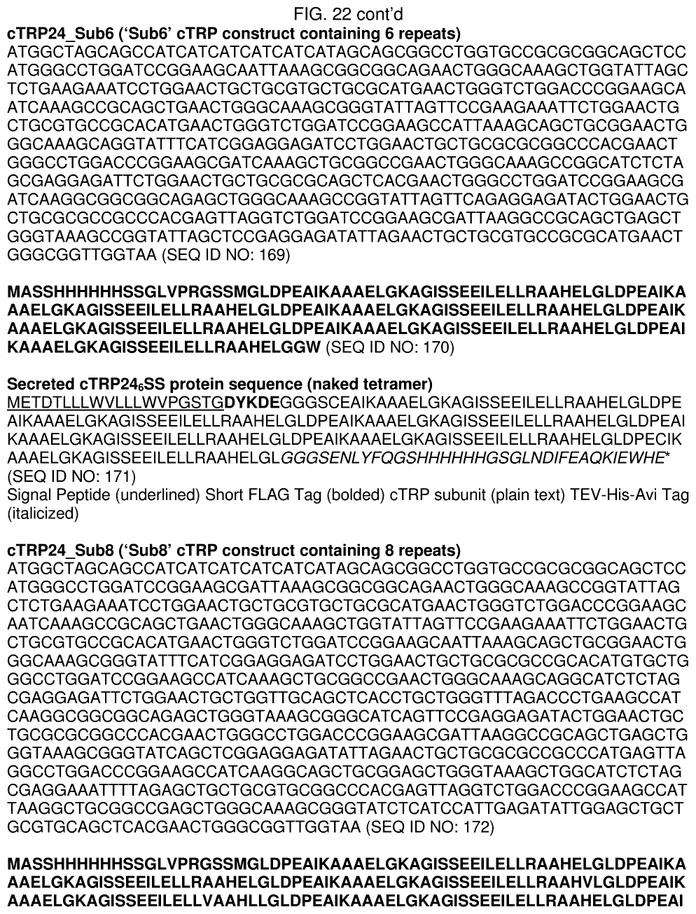

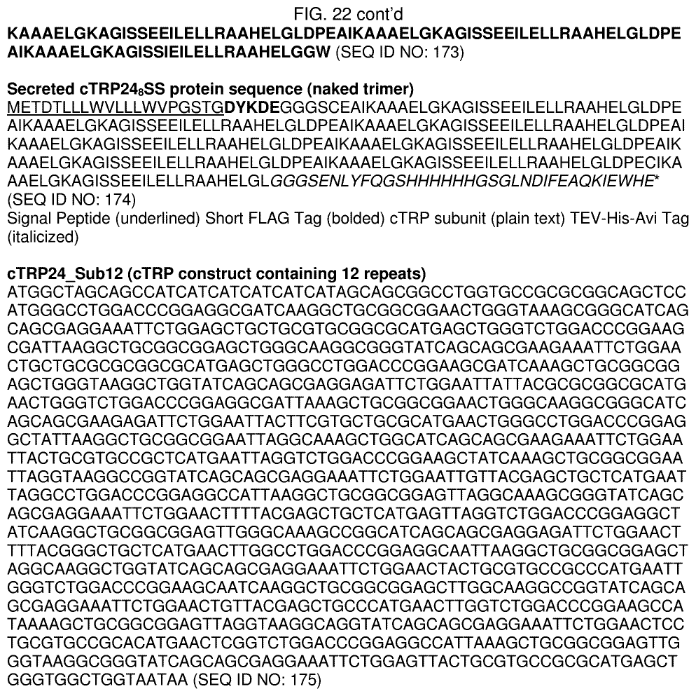

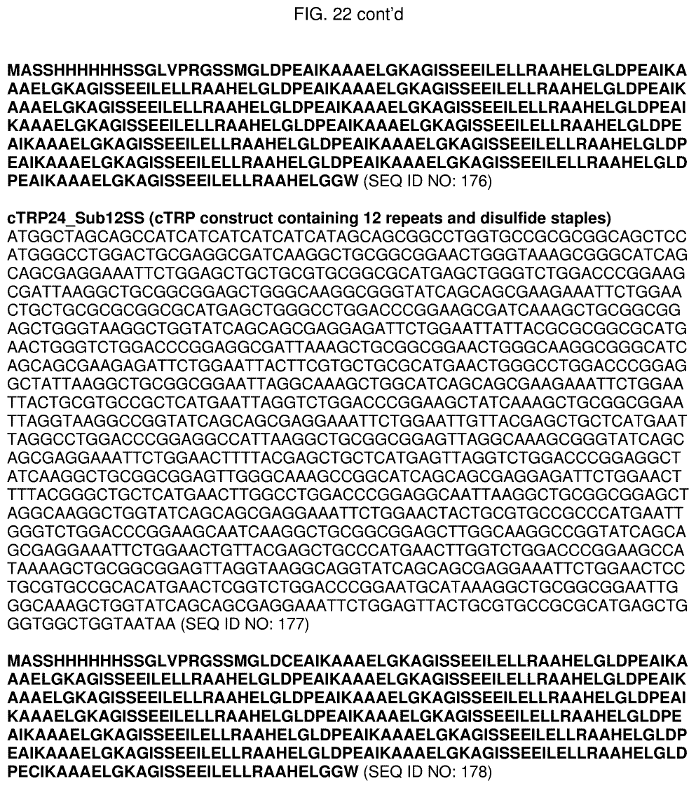

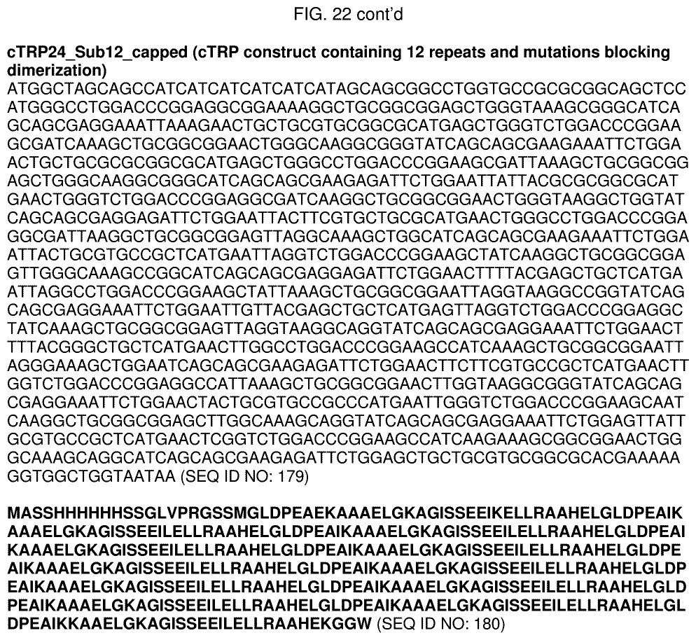

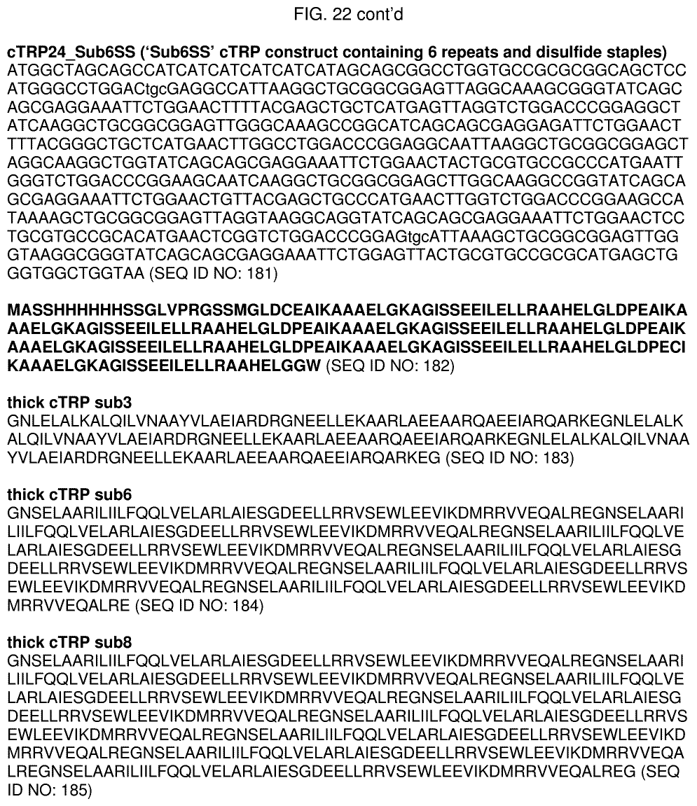

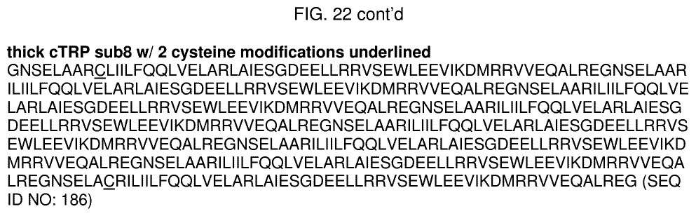

Based on the foregoing, and as stated, in particular embodiments linkers between α helix residues can utilize a GBB format. In particular embodiments, the G residue is glycine. In particular embodiments, the G residue is not isoleucine or valine. In particular embodiments, the B residues are selected from serine, threonine, asparagine, or glutamine. Examples of GBB linkers include GKS; GIT; GTT; GYS; GDK; GDE; NDK; GDR; GDL; and GIS (see, e.g., FIGS. 3B-3K and 22). As will be understood by one of ordinary skill in the art, particular residues that fall within a G or B classification can depend on the particular protein at issue. Therefore, while representative (and common) selection options within these groups are provided, such examples are not exclusive to use of other potential residues. Without being bound by theory, and in particular embodiments, GBB linkers are utilized because they facilitate formation of left-handed proteins. Referring to FIGS. 3B-3K and 22, it is important to note that some cTRP scaffolds are presented as starting with an alpha-helical forming sequence, while others begin with a linker. Due to the circular architecture of cTRPs, these repeat proteins can “begin” or “end” with either segment type at the N- or C-terminus.

In particular embodiments, repetitive α-helical structures joined by linkers can be formed from sequences selected from:

(SEQ ID NO: 55)
GISVEELLKLAKAAYYSGTTVEEAYKLALKL;
(SEQ ID NO: 56)
GISVEELLKLAEAAYYSGTTVEEAYKLALKL;
(SEQ ID NO: 239)
GKSPTEALLKLIAEAKGITETEAKEEAEKALKE;
(SEQ ID NO: 240)
GKSPTEALLKLIAEAKGITSTEAKEEAIKALKE;
(SEQ ID NO: 241)
GKSPTEALLKLIAEAKGITELEAKVLAEKALKE;
(SEQ ID NO: 242)
GKSPTEALLKLIAEAKGITETEAKLEAEKALKE;
(SEQ ID NO: 243)
GKSPTEVLLELIAEASGTTKEEVKEKFLKELSK;
(SEQ ID NO: 244)
GKSPTEVLLELIAEASGTTKEEVKRKFLKELSK;
(SEQ ID NO: 245)
GKSPTEVLLELIAEASGTTKAEVKREFLWELSL;
(SEQ ID NO: 246)
GKSPTEVLLELIAEASGTTKEEVKEKFLAELEK;
(SEQ ID NO: 21)
GKSPTEVLLELIAEASGTTREEVKEKFLKELRK;
(SEQ ID NO: 22)
GKSPTEVLLELIAEASGTTKEEVKEKFLKELSF;
(SEQ ID NO: 23)
GKSPTEVLLELIAEASGTTKEEVKKKFWKELSL;
(SEQ ID NO: 247)
GKSPTEVLLELIAEASGTTKREVKRWFLFELRK;
(SEQ ID NO: 248)
GKSPTEVLLELIAEASGTTKAEVKLKFLFELSF;
(SEQ ID NO: 249)
GKSPTEVLLELIAEASGTTKEEVKEKFLKELFK;
(SEQ ID NO: 250)
GYSTTEALLILIAEASGTTVEQQKQRFKELVKK;
(SEQ ID NO: 251)
GDKTAIAQILAIKASAKGDETELERALRYAKKV;
(SEQ ID NO: 32)
GDKTAIAQILAIKASAKGDETELERALRYAVKV;
(SEQ ID NO: 33)
GDKTAIAQILAIKASAKGDETELEQALRYAKFV;
(SEQ ID NO: 34)
GDKTAIAQILAIKASAKGDELELTRALAYAKKV;
(SEQ ID NO: 35)
GDKTAIAQILAIKASAKGDETELERALRYAKLV;
(SEQ ID NO: 36)
GDKTAIAQILAIKASAKGDETELERALRYAKYV;
(SEQ ID NO: 37)
GDKTAIAQILAIKASAKGDEPELEYALAYAKKV;
(SEQ ID NO: 252)
GDKTAIAQILAIKASAKGDETELERALIFAEAV;
(SEQ ID NO: 253)
NDKTAIAQILAIKASAKGDETELDRALWYAKKV;
(SEQ ID NO: 254)
GDKTAIAQILAIKASAKGDETELERALLYAKKV;
(SEQ ID NO: 255)
GDKTAIAQILAIKASAKGDETELERALAYARLV;
(SEQ ID NO: 256)
GDKTAIAQILAIKASAKGDETELERALRYAEKV;
(SEQ ID NO: 257)
GDKTAIAQILAIKASAKGDEQELEAALIYAKKV;
(SEQ ID NO: 258)
GDKTAIAQILAIKASAKGDETELERALWYAKKV;
(SEQ ID NO: 259)
GDRSAIATAYIALAEYLGDKEALLKAIEIAIKL;
(SEQ ID NO: 260)
GDRSAIAEAYIALARYLGDKEALLKAIEIAIKL;
(SEQ ID NO: 261)
GDKSALAQILAIYASAYGDTTLFLRALKLAKEV;
(SEQ ID NO: 262)
GDLELYIRVLAIVAEAEGDKTKLELALKLALKK;
(SEQ ID NO: 263)
GDLKLYIEVLAIVAEAEGDKTKLELALKLALKK;
(SEQ ID NO: 264)
GDLELYIRVLAIVAKAEGDKTKLELALKLALKK;
(SEQ ID NO: 265)
GDLKLYIEVLAIVAKAEGDKTKLELALKLALKK;
(SEQ ID NO: 45)
GVSLEQALKILKVAAELGTTVEEAVKRALKLKTKL;
(SEQ ID NO: 46)
GVSLEQALKILEVAAELGTTVEEAVKRALKLKTKL;
(SEQ ID NO: 47)
GVSLEQALKILEVAAKLGTTVEEAVKRALKLKTKL;
(SEQ ID NO: 48)
GVSLEQALKILEVAAELGTTVEEAVKRAMKLKTKL;
and
(SEQ ID NO: 44)
LVSLEQALKILKVAAELGTTVEEAVKRALKLKTKL.

Additional examples include SEQ ID NO: 64-68; 75-78; and 82.

SEQ ID NOs: 83,187-205; 16, 24, 38, 49-51, 57, 69-72, and 79 provide exemplary repetitively patterned amino acid sequences that create cTRPs. In particular embodiments, adjacent structural repeats can include sequences that are identical or that have at least 99%, at least 98%, at least 97%, at least 96%, at least 95%, at least 94%, at least 93%, at least 92%, at least 91%, at least 90% or at least 85% sequence identity to the adjacent structural repeat. Methods to determine sequence identity are described below.

As will be understood by one of ordinary skill in the art, variants of the cTRP sequences, that do not alter the circular, handed and repetitive structural nature of the proteins can also be used. Indeed, variants of all protein sequences disclosed herein can be used, so long as the variation does not render the protein unfit for its intended purpose.

In addition to being circular, handed, and structurally repetitive, cTRPs exhibit self-folding, high thermostability, and high solubility. Self-folding means that the cTRPs fold without any need for inclusion of additional folding domains or subunits (e.g., additional protein domains physically appended to the cTRP construct, or independently added protein folding chaperones such as GroEL/GroES or redox-dependent folding cofactors such as thioredoxin). High thermostability means that the proteins retain their overall secondary structure (including alpha-helices) and tertiary structures (defined by their size and shape) at temperatures as high as 95° C. High solubility means that the proteins can be concentrated to levels of 1 mg/mL or higher at physiological pH and salt concentrations without formation of soluble protein aggregates or protein precipitate.

cTRPs can be described as having the formula: (a-b-x-y)ₙ wherein a and x represent linker sequences (e.g., GBB linker sequences), b represents an amino acid sequence that forms an alpha (α) helix, y represents an amino acid sequence that forms a second a helix, n=1 or more, each (a-b-x-y) unit is structurally repetitive to an adjacent (a-b-x-y) unit; the protein is handed (e.g., left-handed); and the N- and C-termini of the protein create a circular architecture.

In certain examples, stability of cTRPs can be increased by adopting one of two approaches. The first approach includes selecting amino acid sequences that form an alpha (α) helix and introducing precisely placed cysteine mutations within the N-terminal and C-terminal segments of the b position of the (a-b-x-y)ₙ formula. These precisely placed cysteine mutations create di-sulfide bonds which serve as a “staple” to fully close the proteins circular architecture. That is, the closed self-assembled cTRPs have an N-terminus and a C-terminus that are physically linked rather than simply constrained by inter-repeat packing geometry.

In particular embodiments, a selected α-helix-forming protein has 13 amino acid residues with a cysteine at position 1 for use in the N-terminal b segment of (a-b-x-y)ₙ. In particular embodiments, a selected α-helix-forming protein has 13 amino acid residues with a cysteine at position 3 for use in the C-terminal b segment of (a-b-x-y)ₙ. In particular embodiments, a selected α-helix-forming protein has 13 amino acid residues with a proline at position 1. The position 1 proline can be modified to cysteine in the N-terminal b segment of (a-b-x-y)ₙ. In particular embodiments, a selected α-helix-forming protein has 13 amino acid residues with an alanine at position 3. The position 3 alanine can be modified to cysteine in the C-terminal b segment of (a-b-x-y)ₙ. These selections and mutations create self-assembled, closed (“stapled”) cTRPs with increased stability over a non-closed circular architecture.

In particular embodiments, a selected α-helix-forming protein has 25 amino acid residues with a cysteine at position 7 for use in the N-terminal b segment of (a-b-x-y)ₙ. In particular embodiments, a selected α-helix-forming protein has 25 amino acid residues with a cysteine at position 5 for use in the C-terminal b segment of (a-b-x-y)ₙ. In particular embodiments, a selected α-helix-forming protein has 25 amino acid residues with an alanine at position 5. The position 5 alanine can be modified to cysteine in the C-terminal b segment of (a-b-x-y)ₙ. In particular embodiments, a selected α-helix-forming protein has 25 amino acid residues with an isoleucine at position 7. The position 7 isoleucine can be modified to cysteine in the N-terminal b segment of (a-b-x-y)ₙ. These selections and mutations create self-assembled, closed cTRPs with increased stability over a non-closed circular architecture (see e.g., FIGS. 3B-3K and 22).

The second approach to creating self-assembling cTRPs with increased stability includes selecting amino acid sequences that form an α helix wherein the selected amino acid sequences each have at least 23 amino acid residues (creating “thick” self-assembling cTRPs).

The first and second approaches can be practiced together to create self-assembling cTRPs that are both closed and thick.

EBV vaccine antigens are presented by a cTRP scaffold. EBV vaccine antigens can be inserted as functional domains, for example, according to the formula (d-a-b-x-y)ₙ, (a-d-b-x-y)ₙ, (a-b-d-x-y)ₙ, (a-b-x-d-y)ₙ or (a-b-x-y-d)ₙ wherein b and y represent linkers, a represents an amino acid sequence that forms an alpha (α) helix, x represents an amino acid sequence that forms a second α helix, d represents an EBV vaccine antigen, and n=1 or more. In certain examples, the d segment EBV antigen can be inserted between residues of a b and/or a y linker sequence.

In particular embodiments, additional linkers can be inserted around a d segment EBV vaccine antigen to further tailor presentation of the vaccine antigen. Linkers can be used that fuse domains together and result in stably expressed, functional proteins. Examples of linkers can be found in Chen et al., Adv Drug Deliv Rev. 2013 Oct. 15; 65(10): 1357-1369. Linkers can be flexible, rigid, or semi-rigid, depending on the desired EBV vaccine antigen presentation. Commonly used flexible linkers include Gly-Ser linkers such as GGSGGGSGGSG (SEQ ID NO: 266), GGSGGGSGSG (SEQ ID NO: 267), GGSGGGSG (SEQ ID NO: 268), GGGGSGGGGS (SEQ ID NO: 269); GGGSGGGS (SEQ ID NO: 270); GGSGGS (SEQ ID NO: 271), GGGSGGGSGGGSGGGS (SEQ ID NO: 290), GGGS (SEQ ID NO: 292), and GS.

In some situations, flexible linkers may be incapable of maintaining a distance or positioning of EBV vaccine antigens needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

The rigidity of protein linkers refers to the degree of flexibility of the protein backbone over the entire length of a short, single chain protein as measured by the average root-mean-square (RMS) (RMS^{fluct}) of all internal torsion angles (Φ, Ψ) over the length of a given single chain protein linker.

RMS^{fluct} of a torsion angle is the standard deviation of the torsion angle value about the time-averaged value in a CHARMm molecular dynamics simulation, wherein RMS^{fluct} is calculated as follows:

$${RMS}^{fluct} = \sqrt{\frac{1}{{N}_{f}} {\sum }_{f} {( {θ}^{f} - {θ}^{ave} )}^{2}}$$

where f refers to the frame number, N is the total number of frames in the trajectory file, and θ^{f} and θ^{ave} are the current value and the average value for the torsion angle, respectively.

“CHARMm” (Chemistry at HARvard Macromolecular Mechanics) refers to a computer simulation engine (see Brooks et al., (1983) J Comp Chem 4: 187-217; MacKerell, et al., (1998) J. Phys. Chem. B 102(18): 3586-3616; and “CHARMM: The Energy Function and Its Parameterization with an Overview of the Program”, by MacKerell et al., in The Encyclopedia of Computational Chemistry, Volume 1, 271-277, by Paul von Raque Schleyer et al., editors (John Wiley & Sons: Chichester, United Kingdom (1998)); and Brooks, et al., (2009) J. Comp. Chem., 30:1545-1615 (2009).

In particular embodiments, the average RMS^{fluct} can be calculated using the formula: (average RMS^{fluct}phi (Φ)+average RMS^{fluct}psi (Ψ))/2. The average RMS fluctuation of all internal backbone torsion angles over the length of the protein can be used to quantify the rigidity of the protein linker. The more rigid the protein is the smaller the average RMS fluctuation should be due to a more limited conformational space accessible to the protein.

In particular embodiments, a rigid protein linker refers to a linker having an average RMS^{fluct} of 25 or less, 20 or less 15 or less when measured using CHARMm modeling over a production run of 200 picoseconds (ps). In particular embodiments, a semi-rigid protein linker refers to a linker having an average RMS^{fluct} of 45-25 when measured using CHARMm modeling over a production run of 200 picoseconds (ps).

As shown in FIG. 10, EBV vaccine antigens can be configured to extend from the “top” and/or the “bottom” of a ring-like cTRP structure. Particular embodiments include different EBV vaccine antigens extending from the top of a cTRP and from the bottom of the same cTRP (FIG. 10). In these “top” and/or the “bottom” embodiments, each “a” linker providing a EBV vaccine antigen on the “top” of a cTRP and each “x” linker position providing a EBV vaccine antigen on the “bottom” of a cTRP (or vice versa). Any portion of a linker loop can serve as the site for insertion of a EBV vaccine antigen so long as the insertion does not impact the integrity of the flanking helices and the folding and function of the inserted EBV vaccine antigen.

As indicated, EBV vaccine antigens can be inserted within an “a” or “x” linker sequence. In particular embodiments, an EBV vaccine antigen can replace an “a” or “x” linker sequence or can replace 1, 2, or 3 residues of an “a” or “x” linker sequence. In particular embodiments, the loops of interest at each position around the top or the bottom of a cTRP can be used as insertion sites for EBV vaccine antigens in a variety of discrete ways, either by interrupting the loops internally (leaving the residues including the first positions and last positions of any loop flanking either side of the cargo) or by inserting adjacent to the loops.

In particular embodiments, the linker sequence is 2 amino acid residues and the EBV vaccine antigen is inserted between the 2 residues. In particular embodiments, the linker sequence is 2 amino acid residues and the EBV vaccine antigen replaces the 1st and/or the 2nd residue of the linker.

In particular embodiments, the linker sequence is 3 amino acid residues and the EBV vaccine antigen is inserted N-terminally after the 1st residue of the linker sequence. In particular embodiments, the linker sequence is 3 amino acid residues and the EBV vaccine antigen is inserted N-terminally after the 2nd residue of the linker sequence. In particular embodiments, the linker sequence is 3 amino acid residues and the EBV vaccine antigen replaces the middle residue of the linker sequence.

In particular embodiments, the linker sequence is 5 amino acid residues and the EBV vaccine antigen replaces the middle residue of linker. In particular embodiments, the linker sequence is 5 amino acid residues and the EBV vaccine antigen is inserted between the 2nd and 3rd residues of the linker or the 3rd and 4th residues of the linker.

In particular embodiments, EBV vaccine antigens can be directly inserted into cTRP sequences for expression and self-assembly. In particular embodiments, antibodies can be captured by EBV vaccine antigens presented on a cTRP scaffold.

(iii) Additional Multimerization Domains. Particular embodiments can utilize additional multimerization domains, such as C4b multimerization domains.

The sequences of a number of C4b domain proteins are available in the art. These include human C4b multimerization domains as well as a number of homologues of human C4b multimerization domain available in the art. There are two types of homologues: orthologues and paralogues. Orthologues are defined as homologous genes in different organisms, i.e. the genes share a common ancestor coincident with the speciation event that generated them. Paralogues are defined as homologous genes in the same organism derived from a gene, chromosome or genome duplication, i.e. the common ancestor of the genes occurred since the last speciation event.

GenBank indicates mammalian C4b multimerization domain homologues in species including chimpanzees, rhesus monkeys, rabbits, rats, dogs, horses, mice, guinea pigs, pigs, chicken, and cattle. Further C4b multimerization domains may be identified by searching databases of DNA or protein sequences, using commonly available search programs such as BLAST.

Particular C4b multimerization domains that can be used include:

| SEQ | |
|---|---|
| ID | |
| NO: | Sequence |
| 330 | SGRAHAGWETPEGCEQVLTGKRLMQCLPNPEDVKMALEVYKLSLEIE |
| | QLELQRDSARQSTLDKELVPR |
| 331 | KKQGDADVCGEVAYIQSVVSDCHVPTAELRTLLEIRKLFLEIQKLKVE |
| | LQGLSKE |
| 332 | ETPEGCEQVLTGKRLMQCLPNPEDVKMALEVYKLSLEIEQLELQRDSA |
| | RQSTLDKEL |
| 333 | WETPEGCEQVLTGKRLMQCLPNPEDVKMALEVYKLSLEIEQLELQRDS |
| | ARQSTLDKEL |
| 334 | CEQVLTGKRLMQCLPNPEDVKMALEVYKLSLEIEQLELQRDSARQSTL |
| | DKEL |
| 335 | ETPEGCEQVLTGKRLMQCLPNPEDVKMALEVYKLSLEIEQLELQRDSA |
| | RQYTLDKEL |
| 336 | ETPEGCEQVLAGKRLMQCLPNPEDVKMALEVYKLSLEIEQLELQRDRA |
| | RQSTLDKEL |
| 337 | ETPEGCEQVLAGKRLMQCLPNPEDVKMALEVYKLSLEIEQLELQRDRA |
| | RQSTWDKEL |
| 338 | EVPEGCEQVQAGRRLMQCLADPYEVKMALEVYKLSLEIELLELQRDKA |
| | RKSSVLRQL |
| 339 | VVPEGCEHILKGRKTMQCLPNPEDVKMALEIYKLSLDIELLELQRDRAK |
| | ESTVQSPV |
| 340 | EVPKDCEHVFAGKKLMQCLPNSNDVKMALEVYKLTLEIKQLQLQIDKAK |
| | HVDREL |
| 341 | EYPEDCEQVHEGKKLMQCLPNLEEIKLALELYKLSLETKLLELQIDKEKK |
| | AKAKYSI |
| 342 | EYPEDCEQVHEGKKLMECLPTLEEIKLALALYKLSLETNLLELQIDKEKK |
| | AKAKYST |
| 343 | EIAEGCEQVLAGRKIMQCLPKPEDVRTALELYKLSLEIKQLEKKLEKEEK |
| | CTPEVQE |
| 344 | EYPEGCEQVVTGRKLLQCLSRPEEVKLALEVYKLSLEIEILQTNKLKKEA |
| | FLLREREKNVTCDFNPE |
| 345 | EYPEGCEQVVTGRKLLKCLSRPEEVKLALEVYKLSLEIALLELQIDKPKD |
| | AS |
| 346 | EVPENCEQVIVGKKLMKCLSNPDEAQMALQLYKLSLEAELLRLQIVKAR |
| | QGS |
| 347 | EASEDLKPALTGNKTMQYVPNSHDVKMALEIYKLTLEVELLQLQIQKEK |
| | HTEAH |
| 348 | VSAEVCEAVFKGQKLLKCLPNAMEVKMALEVYKLSLEIEKLEQEKRKLE |
| | IA |
| 349 | EVPEECKQVAAGRKLLECLPNPSDVKMALEVYKLSLEIEQLEKEKYVKI |
| | QEKFSKKEMKQLTSALH |
| 350 | EVLEDCRIVSRGAQLLHCLSSPEDVHRALKVYKLFLEIERLEHQKEKWI |
| | QLHRKPQSMK |
| 351 | EGPEDCEIVNKGRQLLQCLSSPEDVQRALEVYKLSLEIERLEQQREKRT |
| | SVHRKAHYTKVDGP |
| 352 | EAPEGCEQVLTGRKLMQCLPSPEDVKVALEVYKLSLEIKQLEKERDKL |
| | MNTHQKFSEKEEMKDLFFP |
| 353 | EVPEGCEQVLTGKKLMQCLPNPEDVKMALEVYKLSLEIELLELQIDKAR |
| | QGS |
| 354 | GCEQVLTGKRLMQCLPNPEDVKMALEVYKLSLEIEQLELQRDSARQS |
| 355 | ETPEGCEQVLTGKRLMQCLPNPEDVKMALEVYKLSLEIEQLELQRDSA |
| | RQS |
| 356 | GSEQVLTGKRLMQSLPNPEDVKMALEVYKLSLEIEQLELQRDSARQST |
| | LDKEL |
| 357 | ETPEGCEQVLTGKRLMQCLPNPEDVKMALEIYKLTLEIEQLELQRDSAR |
| | QSTLDKEL |
| 358 | ETPEGCEQVLTGKRLMQCLPNPEDVKMALEIYKLSLEIKQLELQRDSAR |
| | QSTLDKEL |
| 359 | EGCEQILTGKRLMQCLPDPEDVKMALEIYKLSLEIKQLELQRDRARQST |
| | L |
| 360 | ETPEGCEQVLTGKRLMQCLPNPEDVKMALEVYKLSLEIKQLELQRDRA |
| | RQSTLDKEL |
| 361 | EGCEQILTGKRLMQCLPNPEDVKMALEIYKLSLEIEQLELQRDRARQST |
| | LDK |
| 362 | WETPEGCEQVLTGKRLMQCLPNPEDVKMALEVYKLSLEIEQLELQRDS |
| | ARQSTLDKELVPR |

In particular embodiments, the C4b multimerization domain will be a multimerization domain which includes (i) glycine at position 12, (ii) alanine at position 28, (iii) leucines at positions 29, 34, 36, and/or 41; (iv) tyrosine at position 32; (v) lysine at position 33; and/or (vi) cysteine at positions 6 and 18. In particular embodiments, the C4b multimerization domain will be a multimerization domain which includes (i) glycine at position 12, (ii) alanine at position 28, (iii) leucines at positions 29, 34, 36, and 41; (iv) tyrosine at position 32; (v) lysine at position 33; and (vi) cysteine at positions 6 and 18.

C4b multimerization domains can include any of SEQ ID NOs: 330-362 with an N-terminal deletion of at least 1 consecutive amino acid residues (eg. At least 2, 3, 4, 5, 6, 7, 8, 9, 10 consecutive amino acid residues) in length. Additional embodiments can include a C-terminal deletion of at least 1 consecutive amino acid residues (eg. At least 2, 3, 4, 5, 6, 7, 8, 9, 10 consecutive amino acid residues) in length.

Particular C4b multimerization domain embodiments will retain or will be modified to include at least 1 of the following residues: A6; E11; A13; D21; C22; P25; A27; E28; L29; R30; T31; L32; L33; E34; 135; K37; L38; L40; E41; 142; Q43; K44; L45; E48; L49; or Q50. Further embodiments will retain or will be modified to include A6; E11; A13; D21; C22; P25; A27; E28; L29; R30; T31; L32; L33; E34; 135; K37; L38; L40; E41; 142; Q43; K44; L45; E48; L49; and Q50. Particular C4b multimerization domain embodiments will include the amino acid sequence “AELR” (SEQ ID NO: 363).

Particular embodiments can utilize a heptamerization domain such as:

| SEQ ID | |
|---|---|
| NO: | Sequence |
| 364 | AHAGWETPEGCEQVLTGKRLMQCL |
| | PNPEDVKMALEVYKLSLEIEQLEL |
| | QRDSARQSTLDKEL (Human) |
| 365 | SKKQGDADVCGEVAYIQSVVSDCH |
| | VPTEDVKTLLEIRKLFLEIQKLKV |
| | ELQGLSKE (Chicken) |
| 366 | SKKQGDADVCGEVAYIQSVVSDCH |
| | VPTAELRTLLEIRKLFLEIQKLKV |
| | ELQGLSKE (Modified Chicken) |

Additional multimerization domains, such as ferritin multimerization domains may also be used. In certain examples, a multimerization domain, such as SEQ ID NO: 366, is linked to the C-terminus of a vaccine antigen disclosed herein.

Additional examples of multimerization domains include a dimerization and docking domain (DDD) derived from the cAMP-dependent protein kinase (PKA) regulatory subunits paired with an anchoring domain (AD) derived from various A-kinase anchoring proteins (AKAPs) that mediate association with the R subunits of PKA. Additional DDDs and ADs include: the 4-helix bundle type DDD (Newlon, et al. EMBO J. 2001; 20: 1651-1662; Newlon, et al. Nature Struct Biol. 1999; 3: 222-227) domains obtained from p53, DCoH (pterin 4 α carbinolamine dehydratase/dimerization cofactor of hepatocyte nuclear factor 1 α (TCF1)) and HNF-1 (hepatocyte nuclear factor 1) (Rose, et al. Nature Struct Biol. 2000; 7: 744-748). Other AD sequences of potential use may be found in US 2003/0232420A1.

Additional multimerization domains and systems are described in, for example, Hodneland, et al. Proc Natl Acd Sci USA. 2002; 99: 5048-5052; Arakawa et al., J Biol. Chem., 269:27833-27839, 1994; Radziejewski et al., Biochem, 32: 1350, 1993; WO2012001647A2; U.S. Pat. No. 5,821,333; GenBank Accession no. AAF73912.1 (Nishi et al., Mol Cell Biol, 25: 2607-2621, 2005), the SH3 domain of IB1 from GenBank Accession no. AAD22543.1 (Kristensen el al., EMBO J., 25: 785-797, 2006), the PTB domain of human DOK-7 from GenBank Accession no. NP_005535.1 (Wagner et al., Cold Spring Harb Perspect Biol. 5: a008987, 2013), the PDZ-like domain of SATB1 from UniProt Accession No. Q01826 (Galande et al., Mol Cell Biol. August; 21: 5591-5604, 2001), the WD40 repeats of APAF from UniProt Accession No. O14727 (Jorgensen et al., 2009. PLOS One. 4(12):e8463), the PAS motif of the dioxin receptor from UniProt Accession No. I6L9E7 (Pongratz et al., Mol Cell Biol, 18:4079-4088, 1998) and the EF hand motif of parvalbumin from UniProt Accession No. P20472 (Jamalian et al., Int J Proteomics, 2014: 153712, 2014).

(iv) Additional Components. In addition to including EBV vaccine antigens and cTRP scaffolds (including linkers), EBV vaccines disclosed herein can also include signal peptides, tag cassettes, and/or cleavage sites.

A signal peptide, also known as a leader peptide or a signal sequence, is a sequence which functions in directing migration of a nascent protein. A signal peptide can direct the migration of the nascent protein to the cell membrane and/or its extrusion through the membrane. In particular embodiments, a signal peptide is a short peptide sequence having a length of 5-30 amino acids. In particular embodiments, a signal peptide is a short peptide sequence having a length of 5, 10, 15, 20, 25, or 30 amino acids. In particular embodiments, a signal peptide includes a long stretch of hydrophobic amino acids.

In particular embodiments, a signal peptide includes the sequence as set forth in SEQ ID NOs: 280 or 284.

In particular embodiments, EBV vaccines can include one or more tag cassettes. Tag cassettes can be used to detect, enrich for, isolate, track, deplete and/or eliminate recombinant proteins in vitro, in vivo and/or ex vivo. “Tag cassette” refers to a unique synthetic peptide sequence affixed to, fused to, or that is part of a recombinant protein, to which a cognate binding molecule (e.g., ligand, antibody, or other binding partner) is capable of specifically binding where the binding property can be used to detect, enrich for, isolate, track, deplete and/or eliminate the tagged protein.

Tag cassettes that bind cognate binding molecules include, for example, His tag (HHHHHH; SEQ ID NO: 273), Flag tag (DYKDE (SEQ ID NO: 285) or DYKDDDDK (SEQ ID NO: 316)), Xpress tag (DLYDDDDK; SEQ ID NO: 317), Avi tag (GLNDIFEAQKIEWHE; SEQ ID NO: 272), Calmodulin tag (KRRWKKNFIAVSAANRFKKISSSGAL; SEQ ID NO: 318), Polyglutamate tag, HA tag (YPYDVPDYA; SEQ ID NO: 319), Myc tag (EQKLISEEDL; SEQ ID NO: 320), Strep tag (which refers the original STREP® tag (WRHPQFGG; SEQ ID NO: 321), STREP® tag II (WSHPQFEK; SEQ ID NO: 322 (IBA Institut fur Bioanalytik, Germany); see, e.g., U.S. Pat. No. 7,981,632), Softag 1 (SLAELLNAGLGGS; SEQ ID NO: 323), Softag 3 (TQDPSRVG; SEQ ID NO: 324), and V5 tag (GKPIPNPLLGLDST; SEQ ID NO: 325).

Conjugate binding molecules that specifically bind tag cassette sequences disclosed herein are commercially available. For example, His tag antibodies are commercially available from suppliers including Life Technologies, Pierce Antibodies, and GenScript.Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

In particular embodiments, the EBV vaccine includes a cleavage site. A cleavage site can be cleaved by enzymes, for example, proteases (protease cleavage site). A suitable protease for the cleavage of the recombinant protein must be determined.

Proteases specific for certain amino acid sequences are well known to those skilled in the art and include a viral protease including tobacco vein mottling virus (TVMV) protease, Tobacco etch virus (TEV) protease, plum pox virus (PPV) Nla protease, a turnip yellow mosaic virus (TYMV) protease, or to coagulation factor Xa, enterokinase, thrombin, SUMO proteases, ubiquitin proteases, and others. Other proteases with specific cleavage sites may also be used, as known to a person skilled in the art. Sequence specific proteases can be found in a public database in the internet (see for example, MEROPS, the Peptidase Database, http://merops.sangerac.uk). Proteases may be modified to improve activity, solubility, and/or decrease autolysis. Furthermore, proteases may be engineered to be specific for an amino acid sequence which is not recognized by the unmodified protease or recognized with less efficiency. The use of such modified proteases or active portions thereof is also encompassed within this invention.

In particular embodiments, the EBV vaccine (recombinant protein) includes a cleavage site specific for protease. For many proteases, the amino acid sequences for the protease cleavage site are known. In particular embodiments, the cleavage site can be the TEV protease cleavage site (ENLYFQ, ENLYFQG, or ENLYFQS; SEQ ID NO: 293, SEQ ID NO: 326, or SEQ ID NO: 327, respectively) or the TVMV protease cleavage site (ETVRFQS or ETVRFQG: SEQ ID NO: 328 or SEQ ID NO: 329, respectively).

(v) Recombinant Production. In particular embodiments, the EBV vaccines disclosed herein are produced from a gene using a protein expression system. Protein expression systems can utilize DNA constructs (e.g., chimeric genes, expression cassettes, expression vectors, recombination vectors) including a nucleic acid sequence encoding the protein or proteins of interest operatively linked to appropriate regulatory sequences. In particular embodiments, such DNA constructs are not naturally-occurring DNA molecules and are useful for introducing DNA into host-cells to express selected proteins of interest. In particular embodiments, a DNA construct that encodes a vaccine protein can be inserted into cells (e.g., bacterial, mammalian, insect, etc.), which can produce the vaccine protein encoded by the DNA construct.

Operatively linked refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art. See, e.g., Maniatis et al., Molecular Cloning: A Laboratory Manual, Cold Spring Harbor, N. Y., 1982; and Sambrook et al., Molecular Cloning: A Laboratory Manual, Cold Spring Harbor, N. Y., 1989.

Expression control sequences are DNA sequences involved in any way in the control of transcription or translation. Suitable expression control sequences and methods of making and using them are well known in the art. Expression control sequences generally include a promoter. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-2361, 1987. Also, the location of the promoter relative to the transcription start may be optimized. See, e.g., Roberts et al., Proc. Natl. Acad. Sci. USA, 76:760-764, 1979.

The promoter may include, or be modified to include, one or more enhancer elements. In particular embodiments, the promoter will include a plurality of enhancer elements. Promoters including enhancer elements can provide for higher levels of transcription as compared to promoters that do not include them.

For efficient expression, the coding sequences can be operatively linked to a 3′ untranslated sequence. In particular embodiments, the 3′ untranslated sequence can include a transcription termination sequence and a polyadenylation sequence. The 3′ untranslated region can be obtained, for example, from the flanking regions of genes.

In particular embodiments, a 5′ untranslated leader sequence can also be employed. The 5′ untranslated leader sequence is the portion of an mRNA that extends from the 5′ CAP site to the translation initiation codon.

In particular embodiments, a “hisavi” tag can be added to the N-terminus or C-terminus of a gene by the addition of nucleotides coding for the Avitag amino acid sequence, “GLNDIFEAQKIEWHE” (SEQ ID NO: 272), as well as the 6×histidine tag “HHHHHH” (SEQ ID NO: 273). The Avitag avidity tag can be biotinylated by a biotin ligase to allow for biotin-avidin or biotin-streptavidin based interactions for protein purification, as well as for immunobiology (such as immunoblotting or immunofluorescence) using anti-biotin antibodies. The 6×histidine tag allows for protein purification using Ni-²⁺ affinity chromatography. Additional tags are described elsewhere herein.

In particular embodiments, EBV vaccines on a cTRP scaffold can be produced using, for example, human suspension cells and/or the Daedalus expression system as described in Pechman et al., Am J Physiol 294: R1234-R1239, 2008. The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/l of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 2011 (November); 39(21). In some instances, purification by chromatography may not be needed due to the purity of manufacture according the methods described herein.

In particular embodiments, the DNA constructs can be introduced by transfection, a technique that involves introduction of foreign DNA into the nucleus of eukaryotic cells. In particular embodiments, the proteins can be synthesized by transient transfection (DNA does not integrate with the genome of the eukaryotic cells, but the genes are expressed for 24-96 hours). Various methods can be used to introduce the foreign DNA into the host-cells, and transfection can be achieved by chemical-based means including by the calcium phosphate, by dendrimers, by liposomes, and by the use of cationic polymers. Non-chemical methods of transfection include electroporation, sono-poration, optical transfection, protoplast fusion, impalefection, and hydrodynamic delivery. In particular embodiments, transfection can be achieved by particle-based methods including gene gun where the DNA construct is coupled to a nanoparticle of an inert solid which is then “shot” directly into the target-cell's nucleus. Other particle-based transfection methods include magnet assisted transfection and impalefection.

Nucleic acid sequences encoding proteins disclosed herein can be derived by those of ordinary skill in the art. Nucleic acid sequences can also include one or more of various sequence polymorphisms, mutations, and/or sequence variants (e.g., splice variants or codon optimized variants). In particular embodiments, the sequence polymorphisms, mutations, and/or sequence variants do not affect the function of the encoded protein.

Sequence information provided by public databases can be used to identify additional gene and protein sequences that can be used with the systems and methods disclosed.

(vi) Compositions for Administration. EBV therapeutics (vaccines, gene editing systems, viral vectors, or cells modified by gene editing system or viral vectors) can be formulated alone or in combination into compositions for administration to subjects. In particular embodiments, the EBV therapeutics (e.g., EBV vaccines) include immunogenic compositions. An immunogenic composition refers to an agent that stimulates an innate and/or an adaptive immune response in a subject.

Salts and/or pro-drugs of EBV therapeutics can also be used.

A pharmaceutically acceptable salt includes any salt that retains the activity of the EBV therapeutic and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage of an EBV therapeutic or by hydrolysis of a biologically labile group.

In particular embodiments, compositions disclosed herein include an EBV therapeutic of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the EBV therapeutic or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of EBV therapeutic composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts.

Additionally, compositions can be formulated as sustained-release systems utilizing semipermeable matrices of solid polymers including at least one EBV therapeutic. Various sustained-release materials have been established and are well known by those of ordinary skill in the art. Sustained-release systems may, depending on their chemical nature, release one or more EBV therapeutics following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In certain examples, compositions include a vaccine adjuvant. Examples of vaccine adjuvants are described elsewhere herein.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

(vii) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an immunogenic effect. Effective amounts are often administered for research purposes. Effective amounts disclosed herein can cause a statistically-significant effect in an in vitro assay, an animal model or clinical study relevant to the assessment of an infection's development, progression, and/or resolution, as well as the effects of the infection. An immunogenic composition can be provided in an effective amount, wherein the effective amount stimulates an immune response.

A “prophylactic treatment” includes a treatment administered to a subject who does not display signs or symptoms of an infection or displays only early signs or symptoms of an infection such that treatment is administered for the purpose of diminishing or decreasing the risk of developing the infection further. Thus, a prophylactic treatment functions as a preventative treatment against an infection and/or the potential effects of an infection (e.g., IM, a lymphoproliferative disorder (e.g., Hodgkin lymphoma, non-Hodgkin lymphoma, Burkitt lymphoma, T cell lymphoma, lymphoproliferative disease), a carcinoma (e.g., nasopharyngeal and/or gastric) or a smooth muscle tumor).

In particular embodiments, a prophylactic treatment can prevent, delay, or reduce the risk of primary infection with a virus. In particular embodiments, primary infection can refer to when an EBV seronegative individual first becomes infected by EBV and therefore becomes EBV seropositive. In this context, seropositive requires a subject's serum to include different antibodies that bind with multiple and different EBV proteins. Primary infection can result in IM.

In particular embodiments, a prophylactic treatment can be given prior to treatment with an immunosuppressant (such as prior to an organ or cell-based transplant or before chemotherapy or ionizing radiation). In particular embodiments, a prophylactic treatment can prevent or reduce the severity of a lymphoproliferative disorder that may result from EBV primary infection during immunosuppression.

In particular embodiments, prophylactic treatments reduce, delay, or prevent the worsening of an infection. In particular embodiments, a prophylactic treatment can prevent, delay or reduce the severity of EBV reactivation. In particular embodiments, a prophylactic treatment can prevent or reduce the severity of a lymphoproliferative disorder that may result from EBV reactivation during immunosuppression. In particular embodiments, a prophylactic treatment can prevent or reduce the risk of a carcinoma (e.g., nasopharyngeal and/or gastric) or a smooth muscle tumor.

Particular uses of the compositions include use as prophylactic vaccines. Vaccines increase the immunity of a subject against a particular infection. Therefore, “EBV vaccine” can refer to a treatment that increases the immunity of a subject against EBV. Therefore, in particular embodiments, a vaccine may be administered prophylactically, for example to a subject that is immunologically naive (e.g., no prior exposure or experience with EBV). In particular embodiments, a vaccine may be administered therapeutically to a subject who has been exposed to EBV. Thus, a vaccine can be used to ameliorate a symptom associated with EBV, such as a lymphoproliferative disorder. A vaccine can also reduce the risk of a carcinoma (e.g., nasopharyngeal and/or gastric) or a smooth muscle tumor.

In particular embodiments, an EBV vaccine is a therapeutically effective composition including one or more EBV antigens that induces an immune response in a subject against EBV. The skilled artisan will appreciate that the immune system generally is capable of producing an innate immune response and an adaptive immune response. An innate immune response generally can be characterized as not being substantially antigen specific and/or not generating immune memory. An adaptive immune response can be characterized as being substantially antigen specific, maturing over time (e.g., increasing affinity and/or avidity for antigen), and in general can produce immunologic memory. Even though these and other functional distinctions between innate and adaptive immunity can be discerned, the skilled artisan will appreciate that the innate and adaptive immune systems can be integrated and therefore can act in concert.

In particular embodiments, administration of an EBV vaccine can further include administration of one or more adjuvants. The term “adjuvant” refers to material that enhances the immune response to a vaccine antigen and is used herein in the customary use of the term. The precise mode of action is not understood for all adjuvants, but such lack of understanding does not prevent their clinical use for a wide variety of vaccines.

Exemplary vaccine adjuvants, include any kind of Toll-like receptor ligand or combinations thereof (e.g. CpG, Cpg-28 (a TLR9 agonist), polyriboinosinic polyribocytidylic acid (Poly(I:C)), α-galactoceramide, MPLA, Motolimod (VTX-2337, a novel TLR8 agonist developed by VentiRx), IMO-2055 (EMD1201081), TMX-101 (imiquirnod), MGN1703 (a TLR9 agonist), G100 (a stabilized emulsion of the TLR4 agonist glucopyranosyl lipid A), Entolimod (a derivative of Salmonella flagellin also known as CBLB502), Hiltonol (a TLR3 agonist), and Imiquimod), and/or inhibitors of heat-shock protein 90 (Hsp90), such as 17-DMAG (17-dimethylaminoethylamino-17-demethoxygeldanamycin).

In particular embodiments a squalene-based adjuvant can be used. Squalene is part of the group of molecules known as triterpenes, which are all hydrocarbons with 30 carbon molecules. Squalene can be derived from certain plant sources, such as rice bran, wheat germ, amaranth seeds, and olives, as well as from animal sources, such as shark liver oil. In particular embodiments, the squalene-based adjuvant is MF59® (Novartis, Basel, Switzerland). An example of a squalene-based adjuvant that is similar to MF59® but is designed for preclinical research use is Addavax™ (InvivoGen, San Diego, CA). MF59 has been FDA approved for use in an influenza vaccine, and studies indicate that it is safe for use during pregnancy (Tsai T, et al. Vaccine. 2010. 17:28(7):1877-80; Heikkinen T, et al. Am J Obstet Gynecol. 2012. 207(3):177). In particular embodiments, squalene based adjuvants can include 0.1%-20% (v/v) squalene oil. In particular embodiments, squalene based adjuvants can include 5% (v/v) squalene oil.

In particular embodiments the adjuvant alum can be used. Alum refers to a family of salts that contain two sulfate groups, a monovalent cation, and a trivalent metal, such as aluminum or chromium. Alum is an FDA approved adjuvant. In particular embodiments, vaccines can include alum in the amounts of 1-1000μg/dose or 0.1 mg-10 mg/dose.

In particular embodiments, one or more STING agonists are used as a vaccine adjuvant. “STING” is an abbreviation of “stimulator of interferon genes”, which is also known as “endoplasmic reticulum interferon stimulator (ERIS)”, “mediator of IRF3 activation (MITA)”, “MPYS” or “transmembrane protein 173 (TM173)”.

In particular embodiments, STING agonists include cyclic molecules with one or two phosphodiester linkages, and/or one or two phosphorothioate diester linkages, between two nucleotides. This includes (3′,5′)-(3′,5′) nucleotide linkages (abbreviated as (3′,3′)); (3′,5′)-(2′,5′) nucleotide linkages (abbreviated as (3′,2′)); (2′,5′)-(3′,5′) nucleotide linkages (abbreviated as (2′,3′)); and (2′,5′)-(2′,5′) nucleotide linkages (abbreviated as (2′,2′)). “Nucleotide” refers to any nucleoside linked to a phosphate group at the 5′, 3′ or 2′ position of the sugar moiety.

In particular embodiments, STING agonists include c-AIMP; (3′,2′)c-AIMP; (2′,2′)c-AIMP; (2′,3′)c-AIMP; c-AIMP(S); c-(dAMP-dIMP); c-(dAMP-2′FdIMP); c-(2′FdAMP-2′FdIMP); (2′,3′)c-(AMP-2′FdIMP); c-[2′FdAMP(S)-2′FdIMP(S)]; c-[2′FdAMP(S)-2′FdIMP(S)](POM)2; and DMXAA. Additional examples of STING agonists are described in WO2016/145102.

Other immune stimulants can also be used as vaccine adjuvants. Additional exemplary small molecule immune stimulants include TGF-β inhibitors, SHP-inhibitors, STAT-3 inhibitors, and/or STAT-5 inhibitors. Exemplary siRNA capable of down-regulating immune-suppressive signals or oncogenic pathways (such as kras) can be used whereas any plasmid DNA (such as minicircle DNA) encoding immune-stimulatory proteins can also be used.

In particular embodiments, the immune stimulant may be a cytokine and or a combination of cytokines, such as IL-2, IL-12 or IL-15 in combination with IFN-α, IFN-β or IFN-γ, or GM-CSF, or any effective combination thereof, or any other effective combination of cytokines. The above-identified cytokines stimulate T_{H}1 responses, but cytokines that stimulate T_{H}2 responses may also be used, such as IL-4, IL-10, IL-11, or any effective combination thereof. Also, combinations of cytokines that stimulate T_{H}1 responses along with cytokines that stimulate T_{H}2 responses may be used.

“Immune response” refers to a response of the immune system to an EBV antigen disclosed herein. In particular embodiments, an immune response to an EBV antigen can be an innate and/or adaptive response. In particular embodiments, an adaptive immune response can be a “primary immune response” which refers to an immune response occurring on the first exposure of a “naive” subject to an EBV antigen. For example, in the case of a primary antibody response, after a lag or latent period of from 3 to 14 days depending on, for example, the composition, dose, and subject, antibodies to the EBV antigen can be produced. Generally, IgM production lasts for several days followed by IgG production and the IgM response can decrease. Antibody production can terminate after several weeks but memory cells can be produced. In particular embodiments, an adaptive immune response can be a “secondary immune response”, “anamnestic response,” or “booster response” which refer to the immune response occurring on a second and subsequent exposure of a subject to an EBV antigen disclosed herein. Generally, in a secondary immune response, memory cells respond to the EBV antigen and therefore the secondary immune response can differ from a primary immune response qualitatively and/or quantitatively. For example, in comparison to a primary antibody response, the lag period of a secondary antibody response can be shorter, the peak antibody titer can be higher, higher affinity antibody can be produced, and/or antibody can persist for a greater period of time.

In particular embodiments, an immune response against EBV will include antibody production against: the D-I/D-II domain of a gH/gL complex, and/or gB.

A “therapeutic treatment” includes a treatment administered to a subject who displays symptoms or signs of an infection and is administered to the subject for the purpose of diminishing or eliminating those signs or symptoms of the infection or effects of the infection (e.g. IM, a lymphoproliferative disorder (e.g., Hodgkin lymphoma, non-Hodgkin lymphoma, Burkitt lymphoma, T cell lymphoma, lymphoproliferative disease), a carcinoma (e.g., nasopharyngeal and/or gastric) or a smooth muscle tumor). The therapeutic treatment can reduce, control, or eliminate the presence or activity of the infection and/or reduce, control or eliminate side effects of the infection.

In particular embodiments a therapeutic treatment can reduce, control, or eliminate EBV reactivation. In particular embodiments, a reduction in EBV reactivation can be determined by measuring expression of EBV latency genes, wherein detection of fewer latency genes or detection of lower expression levels of latency genes can indicate a reduction in EBV reactivation.

In particular embodiments a therapeutic treatment can reduce, control, or eliminate a primary infection with EBV. In particular embodiments a therapeutic treatment can reduce or eliminate the symptoms of IM.

In particular embodiments, a prophylactic and/or therapeutic treatment can reduce the severity of immunosuppression treatment complications resulting from EBV primary infection of an immunosuppressed individual. In particular embodiments a therapeutically effective treatment to reduce the severity of immunosuppression treatment complications can be given to a pediatric patient. A pediatric patient can refer to patient who is 18 years of age or younger. Pediatric patients are more likely to be EBV seronegative and therefore are at an increased risk of immunosuppression treatment complications from EBV primary infection, as compared to adult patients.

In particular embodiments a therapeutic treatment can eliminate or reduce the severity of a lymphoproliferative disorder. Elimination of or reduced severity of a lymphoproliferative disorder can be indicated by a reduction in lymphocyte count in an individual with the lymphoproliferative disorder.

In particular embodiments a therapeutic treatment can eliminate or reduce the risk or severity of a carcinoma (e.g., nasopharyngeal and/or gastric) or a smooth muscle tumor.

Function as an effective amount, prophylactic treatment or therapeutic treatment are not mutually exclusive, and in particular embodiments, administered dosages may accomplish more than one treatment type.

In particular embodiments, therapeutically effective amounts provide anti-infection effects. Anti-infection effects include a decrease in the number of infected cells, a decrease in volume of infected tissue, reduced infection-associated lymphoproliferation, reduced occurrence of infection-associated carcinoma (e.g., nasopharyngeal and/or gastric) or a smooth muscle tumor, and/or reduction or elimination of a symptom associated with the treated infection.

Effects of EBV infection can include infectious mononucleosis (IM), a lymphoproliferative disorder, and/or a carcinoma (e.g., nasopharyngeal and/or gastric) or a smooth muscle tumor. IM is an illness caused by primary infection with EBV and symptoms of IM can include fever, swollen lymph nodes, swollen tonsils, loss of appetite, fatigue, abdominal pain, and/or spleen enlargement. In particular embodiments, therapeutically effective amounts provide anti-IM effects. Anti-IM effects include a reduction or elimination of an IM symptom.

Lymphoproliferative disorders can refer to the uncontrolled division of lymphocytes. Subjects with compromised immune systems are at increased risk of developing a lymphoproliferative disorder. Examples of lymphoproliferative disorders include Hodgkin lymphoma, non-Hodgkin lymphoma, Burkitt lymphoma, T cell lymphomas, follicular lymphoma, chronic lymphocytic leukemia, acute lymphoblastic leukemia, hairy cell leukemia, B-cell lymphomas, multiple myeloma, Waldenstrom's macroglobulinemia, Wiskott-Aldrich syndrome, lymphocyte-variant hypereosinophilia, post-transplant lymphoproliferative disorder, and autoimmune lymphoproliferative syndrome. In particular embodiments, lymphoproliferative disorders include lymphoproliferative disease, which is an EBV-related lymphoma that occurs in 1-20% of bone marrow and solid organ transplant recipients.

Symptoms of lymphoproliferative disorders can include adenopathy, splenomegaly, and/or an abnormally high lymphocyte count in a subject's blood sample. In particular embodiments, therapeutically effective amounts provide anti-lymphoproliferative disorder effects. Anti-lymphoproliferative disorder effects include a reduction or elimination of a lymphoproliferative disorder or a symptom of a lymphoproliferative disorder.

In particular embodiments, the term “carcinoma” designates any disease involving unregulated proliferation of epithelial cells, and which may result in unregulated cell growth, lack of differentiation, tumors formation, local tissue invasion, and/or metastasis formation. Nasopharyngeal carcinoma (NPC) is a malignant tumor arising from the epithelial lining of the nasopharynx, which is located behind the nose and above the back of the throat. NPC differs significantly from other cancers of the head and neck, based on its causes, occurrence, clinical behavior, and treatment options. NPC is consistently associated with EBV and is the third most frequent virus-associated malignancy in humans.

Gastric carcinomas include gastric cancer, including intestinal and diffuse gastric adenocarcinoma, gastrointestinal stromal tumor (GIST), gastrointestinal leiomyosarcoma, gastrointestinal carcinoid, gastrointestinal lymphoma, esophagogastric adenocarcinoma (OGA), and colorectal carcinoma.

Leiomyomas are tumors composed of smooth muscle cells which can range from clearly benign leiomyoma (fibroids) to malignant leiomyosarcoma. Intermediate variants have also been identified and are termed “smooth muscle tumors of uncertain malignant potential”. These may include cellular leiomyomas. Leiomyomas and leiomyosarcomas are most prevalent in the uterine smooth muscle, however, they can occur in any organ system which possesses smooth muscle. The second highest incidence of occurrence for these tumor types is in the gastrointestinal tract. Leiomyomas represent one of the most common benign tumors of the stomach, while gastric leiomyosarcomas represent 2% of all malignant tumors that occur in the stomach.

In particular embodiments, therapeutically effective amounts provide anti-carcinoma (e.g., anti-NPC; anti-gastric carcinoma) or anti-smooth muscle tumor effects which can include reducing the risk or occurrence of a carcinoma or smooth muscle tumor, limiting the further development of the carcinoma or smooth muscle tumor (e.g., tumor growth and/or metastasis), reversing the severity of the carcinoma or smooth muscle tumor, or other beneficial clinical outcomes, as understood one of ordinary skill in the art. For example, beneficial clinical outcomes include loss of detectable tumor (complete response), decrease in tumor size (partial response, PR), tumor growth or cell number increase arrest (stable disease, SD), enhancement of anti-tumor immune response, and/or relief, to some extent, of one or more symptoms associated with a carcinoma or smooth muscle tumor; increase in the length of survival following treatment; and/or decreased mortality at a given point of time following treatment.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of infection, stage of infection, effects of infection (e.g., IM, lymphoproliferative disorders), previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The pharmaceutical compositions described herein can be administered by, without limitation, injection, inhalation, infusion, perfusion, lavage or ingestion. Routes of administration can include intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual administration and more particularly by intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual injection.

(viii) Kits. Also disclosed herein are kits including one or more containers including one or more of the EBV vaccines, modified cells, and/or compositions and/or adjuvants described herein. Associated with such container(s) can be a notice in the form prescribed by a governmental agency regulating the manufacture, use, or sale of pharmaceuticals or biological products, which notice reflects approval by the agency of manufacture, use, or sale for human administration.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

(ix) Exemplary Embodiments.
1. An EBV vaccine including the sequence as set forth in SEQ ID NOs: 304, 305, 306, 307, 308, 309, 310, 311, 312, or 313 or a sequence having at least 90% sequence identity to the sequence as set forth in SEQ ID NOs: 304, 305, 306, 307, 308, 309, 310, 311, 312, or 313.
2. The EBV vaccine of embodiment 1 including the sequence as set forth in SEQ ID NO: 307 or SEQ ID NO: 308 or a sequence having at least 90% sequence identity to the sequence as set forth in SEQ ID NO: 307 or SEQ ID NO: 308.
3. The EBV vaccine of embodiment 1, including the sequence as set forth in SEQ ID NOs: 294, 295, 296, 297, 298, 299, 300, 301, 302, or 303 or a sequence having at least 90% sequence identity to the sequence as set forth in SEQ ID NOs: 294, 295, 296, 297, 298, 299, 300, 301, 302, or 303.
4. The EBV vaccine of embodiment 1, including the sequence as set forth in SEQ ID NO: 297 or SEQ ID NO: 298 or a sequence having at least 90% sequence identity to the sequence as set forth in SEQ ID NO: 297 or SEQ ID NO: 298.
5. The EBV vaccine of embodiment 1, including the sequence as set forth in SEQ ID NOs: 6, 7, 8, 9, 10, 11, 12, 13, 14, or 15 or a sequence having at least 90% sequence identity to the sequence as set forth in SEQ ID NOs: 6, 7, 8, 9, 10, 11, 12, 13, 14, or 15.
6. The EBV vaccine of embodiment 1, including the sequence as set forth in SEQ ID NO: 9 or SEQ ID NO: 10, or a sequence having at least 90% sequence identity to the sequence as set forth in SEQ ID NO: 9 or SEQ ID NO: 10.
7. An EBV vaccine including a circular, handed protein including at least three α-helical structures wherein each α-helical structure includes an outer α helix and an inner α helix joined by a flexible linker and wherein each α-helical structure has at least 95% sequence identity with an adjacent α-helical structure.
8. The EBV vaccine of embodiment 7, having the formula: (d-a-b-x-y)ₙ, (a-d-b-x-y)ₙ, (a-b-d-x-y)ₙ, (a-b-x-d-y)ₙ or (a-b-x-y-d)ₙ wherein
   b and y each represent a linker,
   a represents an amino acid sequence that forms an alpha (α) helix,
   x represents an amino acid sequence that forms a second α helix,
   d represents an EBV vaccine antigen, and
   the protein is handed.
9. The EBV vaccine of embodiment 7, having the formula: (d-a-b-x-y)ₙ, (a-d-b-x-y)ₙ, (a-b-d-x-y)ₙ, (a-b-x-d-y)ₙ or (a-b-x-y-d)ₙ wherein
   a and x each represent a linker,
   b represents an amino acid sequence that forms an alpha (α) helix,
y represents an amino acid sequence that forms a second α helix,
   d represents an EBV vaccine antigen, and
   the protein is handed.
10. The EBV vaccine of embodiment 9, wherein n=3, 6, 9, 12, or 24.
11. The EBV vaccine of embodiments 9 or 10, wherein each b and y segment has 13 amino acid residues.
12. The EBV vaccine of any one of embodiments 9-11, wherein each a and x linker represent a 2, 3, 4, or 5 amino acid linker.
13. The EBV vaccine of any one of embodiments 9-12, wherein the N-terminal b segment has 13 amino acids and a cysteine at position 1 and the C-terminal segment b segment has 13 amino acids and a cysteine at position 3.
14. The EBV vaccine of embodiment 13, wherein the N-terminal b segment has the sequence as set forth in SEQ ID NO: 80, 81, 91, 92, 93, 94, 95, 96, 97, 98, 99, 100, or 101.
15. The EBV vaccine of embodiment 13, wherein the N-terminal b segment has the sequence as set forth in SEQ ID NO: 95.
16. The EBV vaccine of any one of embodiments 9-15, wherein the C-terminal b segment has the sequence as set forth in SEQ ID NO. 80, 81, 89, 91, 92, 93, 94, 99, 103, 104, or 105.
17. The EBV vaccine of any one of embodiments 9-15, wherein the C-terminal b segment has the sequence as set forth in SEQ ID NO: 89.
18. The EBV vaccine of any one of embodiments 9-17, including the sequence as set forth in SEQ ID NO: 283 or SEQ ID NO: 291 or having at least 90% sequence identity to the sequence as set forth in SEQ ID NO: 283 and/or SEQ ID NO: 291.
19. The EBV vaccine of any one of embodiments 9-18, wherein the EBV vaccine antigen includes gH or a fragment of gH.
20. The EBV vaccine of any one of embodiments 9-19, wherein the EBV vaccine antigen includes gL or a fragment of gL.
21. The EBV vaccine of any one of embodiments 9-20, wherein the EBV vaccine antigen includes gp42 or a fragment of gp42.
22. The EBV vaccine of any one of embodiments 9-21, wherein the EBV vaccine antigen includes gB or a fragment of gB.
23. The EBV vaccine of any one of embodiments 9-18, wherein the EBV vaccine antigen has the sequence as set forth in SEQ ID NO: 1, 2, 3, 4, or 5 or has a sequence with at least 90% sequence identity to the sequence as set forth in SEQ ID NO: 1, 2, 3, 4, or 5.
24. The EBV vaccine of any one of embodiments 9-18, wherein the EBV vaccine antigen has the sequence as set forth in SEQ ID NO: 281, 282, 286, 287, 288, or 289 or has a sequence with at least 90% sequence identity to the sequence as set forth in SEQ ID NO: 281, 282, 286, 287, 288, or 289.
25. The EBV vaccine of any one of embodiments 9-24, wherein b and y have at least 98% sequence identity.
26. The EBV vaccine of any one of embodiments 9-25, wherein the linkers are flexible linkers.
27. The EBV vaccine of embodiment 26, wherein the flexible linkers include GBB linkers.
28. The EBV vaccine of embodiments 26 or 27, wherein the flexible linkers have the sequence GD, GN, GS, GT, GLD, GLN, GLS, GLT, GID, GIN, GIT, GIS, GVD, GVN, GVT, GVS, GTD, GTN, GTT, GTS, GKS, GYS, GNS, LPHD (SEQ ID NO: 274), NPND (SEQ ID NO: 275), DPKD (SEQ ID NO: 276), GLEPD (SEQ ID NO: 277), GVSLD (SEQ ID NO: 278), or GVLPD (SEQ ID NO: 279).
29. The EBV vaccine of embodiments 26 or 27, wherein the flexible linkers have the sequence GLD, GLN, GLS, GLT, GID, GIN, GIT, GIS, GVD, GVN, GVT, GVS, GTD, GTN, GTT, GTS, GKS, GYS, or GNS.
30. The EBV vaccine of embodiments 26 or 27, wherein the flexible linkers have the sequence GKS; GIT; GTT; GYS; GDK; GDE; NDK; GDR; GDL; or GIS.
31. The EBV vaccine of any one of embodiments 9-30, having at least two EBV vaccine antigens wherein each EBV vaccine antigen is inserted into the sequence of the protein between an outer α helix of the protein and an adjacent inner α helix of the protein.
32. The EBV vaccine of embodiment 31, wherein at least one of the EBV vaccine antigens has the sequence as set forth in SEQ ID NO: 1, 2, 3, 4, 5, 281, 282, 286, 287, 288, or 289 or has a sequence with at least 90% sequence identity to the sequence as set forth in SEQ ID NO: 1, 2, 3, 4, 5, 281, 282, 286, 287, 288, or 289.
33. The EBV vaccine of any one of embodiments 9-32, further including a EBV vaccine antigen (d) that replaces 1, 2, or 3 residues of an a or x linker sequence.
34. The EBV vaccine of any one of embodiments 9-33, further including at least two EBV vaccine antigens (d) inserted in a (a-b-x-y) unit within or adjacent to an a or x linker sequence.
35. The EBV vaccine of any one of embodiments 9-34, further including a flexible, rigid, or semi-rigid linker adjacent to an EBV vaccine antigen.
36. The EBV vaccine of embodiment 35, wherein the linker is a flexible linker.
37. The EBV vaccine of embodiment 35, wherein the linker has the sequence GS or the sequence as set forth in SEQ ID NOs: 269, 290, or 292.
38. An EBV vaccine including (i) an antigen selected from gH, a fragment of gH, gL, a fragment of gL, gp42, a fragment of gp42, gB, and a fragment of gB and (ii) a C4b multimerization domain.
39. The EBV vaccine of embodiment 38, wherein the antigen has the sequence as set forth in SEQ ID NO: 1, 2, 3, 4, 5, 281, 282, 286, 287, 288, or 289 or has a sequence with at least 90% sequence identity to the sequence as set forth in SEQ ID NO: 1, 2, 3, 4, 5, 281, 282, 286, 287, 288, or 289.
40. The EBV vaccine of embodiment 39, wherein the EBV vaccine antigen includes gH and gL.
41. The EBV vaccine of any one of embodiments 38-40, wherein the EBV vaccine is a heptamer.
42. The EBV vaccine of any one of embodiments 38-41, wherein the multimerization domain is a heptamerization domain including the sequence as set forth in SEQ ID NOs: 364, 365, or 366.
43. The EBV vaccine of any one of embodiments 38-42, wherein the multimerization domain is linked to the C terminus of the antigen.
44. The EBV vaccine of any one of embodiments 38-43, wherein the antigen includes gH and gL, the multimerization domain includes SEQ ID NOs: 364, 365, or 366, and the multimerization domain is linked to the C terminus of the gH.
45. A nucleotide sequence encoding an EBV vaccine of embodiment 1.
46. A cell including a nucleotide sequence of embodiment 45.
47. A composition formulated for administration to a subject including an EBV vaccine of embodiment 1, a nucleotide sequence of embodiment 47, or a cell of embodiment 46.
48. The composition of embodiment 47, further including an adjuvant.
49. The composition of embodiment 48, wherein the adjuvant includes alum, a squalene-based adjuvant, a STING agonist, or a liposome-based adjuvant.
50. A method of stimulating an anti-EBV immune response in a subject including administering to the subject a therapeutically effective amount of a composition of embodiment 47 to the subject, thereby stimulating an EBV immune response in the subject.
51. The method of embodiment 50, wherein the subject is EBV seropositive.
52. The method of embodiment 50, wherein the subject is an EBV seronegative subject, and wherein the therapeutically effective amount reduces the risk of EBV infection.
53. The method of embodiment 52, wherein the EBV seronegative subject is a pediatric patient.
54. The method of any one of embodiments 50-53, wherein the administering is prior to treatment with an immunosuppressant.
55. The method of any one of embodiments 50-54, wherein the subject is a transplant patient.
56. The method of any one of embodiments 50-55, wherein the therapeutically effective amount reduces the risk or severity of infectious mononucleosis (IM).
57. The method of any one of embodiments 50-56, wherein the method treats the subject for an EBV infection.
58. The method of embodiment 57, wherein the treating reduces or eliminates infectious mononucleosis (IM) and/or symptoms of IM.
59. The method of embodiment 57, wherein the treating (i) reduces or eliminates a lymphoproliferative disorder and/or symptoms of the lymphoproliferative disorder, (ii) reduces the risk or occurrence of a carcinoma, and/or (iii) reduces the risk or occurrence of a smooth muscle tumor.
60. The method of embodiment 59, wherein lymphoproliferative disorder includes Hodgkin lymphoma, non-Hodgkin lymphoma, Burkitt lymphoma, T cell lymphoma, or lymphoproliferative disease.
61. The method of embodiment 60, wherein the carcinoma includes nasopharyngeal carcinoma or gastric carcinoma.
62. The method of any one of embodiments 50-61, further including administering a second therapeutically effective amount of a composition of embodiment 49 to the subject.
63. The method of any one of embodiments 50-62, further including administering a third therapeutically effective amount of a composition of embodiment 49 to the subject.

(x) Experimental Examples. Example 1. Immunization with a self-assembling nanoparticle vaccine displaying EBV gH/gL protects humanized mice against lethal viral challenge.

Summary. Epstein-Barr virus (EBV) is a cancer-associated pathogen responsible for 165,000 deaths annually. EBV is also the etiological agent of infectious mononucleosis and is linked to multiple sclerosis and rheumatoid arthritis. Thus, an EBV vaccine would have a significant global health impact. EBV is orally transmitted and has tropism for epithelial and B cells. Therefore, a vaccine would need to prevent infection of both in the oral cavity. Passive transfer of monoclonal antibodies against the gH/gL glycoprotein complex prevent experimental EBV infection in humanized mice and rhesus macaques, suggesting that gH/gL is an attractive vaccine candidate. Here, the immunogenicity of several gH/gL nanoparticle vaccines were evaluated. All display superior immunogenicity relative to monomeric gH/gL.

Introduction. Epstein-Barr virus (EBV) is one of the most common human viruses. It is a herpesvirus with tropism for both B cells and epithelial cells and is associated with several malignancies of these two cell types including Hodgkin lymphoma, Burkitt lymphoma, diffuse large B cell lymphoma, post-transplant lymphoproliferative disease, nasopharyngeal carcinoma and gastric carcinoma (Shannon-Lowe, C and A Rickinson. (2019) Frontiers in oncology 9, 713; Taylor, G. S., et al. (2015) Annual review of immunology 33, 787-821; Cohen, J. I. (2018). Advances in experimental medicine and biology 1045, 477-493; and Kutok, J. L., and Wang, F. (2006). Annual review of pathology 1, 375-404). It is estimated that EBV is responsible for 265,000 new cases of cancer and 164,000 cancer deaths globally per year (Shannon-Lowe, C and A Rickinson. (2019) Frontiers in oncology 9, 713; Khan, G., and Hashim, M. J. (2014). Infectious agents and cancer 9, 38; Cohen, J. I. et al. (2011) Science translational medicine 3, 107fs107; and Khan, G., et al. (2020). BMJ Open 10, e037505). EBV is also the causative agent of infectious mononucleosis (IM) and is linked to multiple sclerosis and rheumatoid arthritis (Levin, L. I., et al. (2010). Annals of neurology 67, 824-830; Handel, A. E., et al. (2010). PloS one 5; Thacker, E. L., et al. (2006). Annals of neurology 59, 499-503; Munger, K. L., et al. (2011). Mult Scler 17, 1185-1193; Balandraud, N., and Roudier, J. (2018). Joint, bone, spine: revue du rhumatisme 85, 165-170; Angelini, D. F., et al. (2013). PLoS Pathog 9, e1003220; and Bjornevik, K., et al. (2022). Science). Thus, a vaccine that prevents EBV infection and/or associated pathologies would have a significant global health impact (Shannon-Lowe, C and A Rickinson. (2019) Frontiers in oncology 9, 713; Cohen, J. I. et al. (2011) Science translational medicine 3, 107fs107; and Ainsworth, C. (2018). Nature 563, S52-s54).

EBV is orally transmitted and both B cells and epithelial cells are present in the oropharynx. Thus, an effective vaccine would likely need to prevent or severely limit infection in both cell types (Taylor, G. S., et al. (2015) Annual review of immunology 33, 787-821; and Rickinson, A. B., et al. (2014). Trends Immunol 35, 159-169). The dual tropism of EBV infection is accomplished through the orchestrated function of multiple glycoproteins (Connolly, S. A., et al. (2021). Nat Rev Microbiol 19, 110-121). gH, gL and gB constitute the core fusion machinery and are essential for viral entry irrespective of cell type. gB is a transmembrane fusion protein that promotes the merger of the viral and host membranes (Backovic, M., et al. (2009). Proceedings of the National Academy of Sciences of the United States of America 106, 2880-2885). gB activity depends on the heterodimeric gH/gL complex, which regulates fusion and is essential for infection (Oda, T., et al. (2000). Virology 276, 52-58; Stampfer, S. D., and Heldwein, E. E. (2013). Current opinion in virology 3, 13-19; Haddad, R. S., and Hutt-Fletcher, L. M. (1989). J Virol 63, 4998-5005; and Mohl, B. S., et al. (2016). Molecules and cells 39, 286-291). Epithelial cell infection is initiated by the binding of the viral BMRF-2 protein to β1 integrins on the cell surface (Tugizov, S. M., et al. (2003). Nature medicine 9, 307-314). Following attachment, binding of gH/gL to one or more cell surface receptors is thought to induce a conformational change that triggers gB activation. avβ6, and avβ8 integrins, neuropilin 1, non-muscle myosin heavy chain IIA and the ephrin A2 receptor have all been implicated as gH/gL receptors (Chesnokova, L. S., et al. (2009). Proceedings of the National Academy of Sciences of the United States of America 106, 20464-20469.; Chen, J., et al. (2018). Nat Microbiol 3, 172-180; Zhang, H., et al. (2018) Nat Microbiol 3, 1-8; Wang, H. B., et al. (2015). Nat Commun 6, 6240; Xiong, D., et al. (2015). Proc Natl Acad Sci USA 112, 11036-11041; and Su, C., et al. (2020). Nat Commun 11, 5964).

Viral attachment to B cells is mediated by gp350, which binds to complement receptors (CR) 1 and 2 (Tanner, J., et al. (1987). Cell 50, 203-213; Ogembo, J. G., et al. (2013). Cell reports 3, 371-385; and Nemerow, G. R., et al. (1987). J Virol 61, 1416-1420). The triggering of gB during B cell entry depends on the tripartite complex of gH/gL and the viral glycoprotein gp42. Binding of gp42 to the B chain of human leukocyte antigen class II leads to activation of gB through the gH/gL/gp42 complex (Spriggs, M. K., et al. (1996). Journal of virology 70, 5557-5563; Sathiyamoorthy, K., et al. (2014). PLoS pathogens 10, e1004309; and Haan, K. M. et al. (2000). J Virol 74, 2451-2454).

Neutralizing antibodies are the correlate of protection for most effective vaccines (Plotkin, S. A. (2010). Clinical and vaccine immunology: CVI 17, 1055-1065; and Gilbert, P. B., et al. (2022). Science 375, 43-50). It is therefore likely that they will be an important component of an immune response elicited by an EBV vaccine. Serum from naturally infected individuals can neutralize EBV infection of B cells and epithelial cells (Tugizov, S. M., et al. (2003). Nature medicine 9, 307-314; Sashihara, J., et al. (2009). Virology 391, 249-256; Miller, G., et al. (1972). The Journal of infectious diseases 125, 403-406; and Moss, D. J., and Pope, J. H. (1972). The Journal of general virology 17, 233-236), and all the viral proteins involved in viral entry are targeted by neutralizing antibodies (Tugizov, S. M., et al. (2003). Nature medicine 9, 307-314; Thorley-Lawson, D. A., and Poodry, C. A. (1982). Journal of virology 43, 730-736; Xiao, J., et al. (2009) Virology 393, 151-159; and Bu, W et al. (2019). Immunity 50, 1305-1316.e1306). To date, most EBV subunit vaccine efforts have focused on gp350. gp350 is capable of adsorbing most of the serum antibodies that neutralize EBV infection of B cells (Thorley-Lawson, D. A., and Poodry, C. A. (1982). Journal of virology 43, 730-736; and Bu, W et al. (2019). Immunity 50, 1305-1316.e1306).

Mechanistically, neutralizing anti-gp350 monoclonal antibodies (mAbs) block the gp350-CR1/CR2 interaction (Ogembo, J. G., et al. (2013). Cell reports 3, 371-385; Hoffman, G. J., et al. (1980). PNAS 77, 2979-2983; Tanner, J., et al. (1988). J Virol 62, 4452-4464; Mutsvunguma, L. Z., et al. (2019). Virology 536, 1-15; and Szakonyi, G., et al. (2006). Nat Struct Mol Biol 13, 996-1001). However, antibodies against gp350 are ineffective at inhibiting EBV infection of CR⁻ epithelial cells and can enhance infection of this cell type (Tugizov, S. M., et al. (2003). Nature medicine 9, 307-314; Molesworth, S. J., et al. (2000). Journal of virology 74, 6324-6332; and Turk, S. M., et al. (2006). Journal of virology 80, 9628-9633). Passive transfer of a neutralizing anti-gp350 mAb protected one of three macaques against high-dose experimental infection with rhesus lymphocryptovirus, the EBV ortholog that infects macaques (Mühe, J., et al. (2021). Cell reports. Medicine 2, 100352) indicating that gp350 antibodies could be protective in vivo. A phase II trial of a gp350 vaccine failed to protect against EBV despite decreasing the incidence of symptomatic infectious mononucleosis by 78% (Sokal, E. M., et al. (2007). The Journal of infectious diseases 196, 1749-1753). In light of these results, it has been suggested that a gp350 vaccine could be improved upon with the inclusion of additional viral proteins (Cohen, J. I., et al. (2013). Vaccine 31 Suppl 2, B194-196). Alternatively, it is possible that a vaccine targeting non-gp350 viral proteins could be more efficacious.

gH/gL is a promising antigen for vaccine development. Anti-gH/gL antibodies account for most serum antibodies that neutralize EBV infection of epithelial cells, but only a small fraction of antibodies that neutralize infection of B cells (Bu, W et al. (2019). Immunity 50, 1305-1316.e1306). Only a handful of anti-gH/gL monoclonal antibodies (mAbs) have been identified, all of which neutralize EBV infection of epithelial cells with comparable potency, but most have weak, or no neutralizing activity against EBV infection of B cells (Molesworth, S. J., et al. (2000). Journal of virology 74, 6324-6332; Chesnokova, L. S., and Hutt-Fletcher, L. M. (2011). J Virol 85, 13214-13223; Sathiyamoorthy, K., et al. (2017). Proceedings of the National Academy of Sciences 114, E8703-E8710; Snijder, J., et al. (2018). Immunity 48, 799-811.e799; Sathiyamoorthy, K., et al. (2016). Nature communications 7, 13557; Li, Q., et al. (1995). J Virol 69, 3987-3994; and Zhu, Q. Y., et al. (2021). Nat Commun 12, 6624). The isolation and characterization of AMMO1 has been previously described, an anti-gH/gL monoclonal antibody (mAb) which potently neutralizes EBV infection of epithelial cells and B cells in vitro by binding to a discontinuous epitope on gH/gL (Snijder, J., et al. (2018). Immunity 48, 799-811.e799). The 769B10 mAb also neutralizes EBV infection of both cell types and binds to an epitope that overlaps with AMMO1, confirming this is a critical site of vulnerability on EBV (Bu, W et al. (2019). Immunity 50, 1305-1316.e1306). Passive transfer of AMMO1 severely limits viral infection following high-dose experimental EBV challenge in humanized mice and protects rhesus macaques against oral challenge with RhLCV if present at adequate levels at the time of challenge (Zhu, Q. Y., et al. (2021). Nat Commun 12, 6624; and Singh, S., et al. (2020). Medicine 1). These studies provide proof of concept that anti-gH/gL antibodies can protect against EBV infection and indicate that a gH/gL-based vaccine capable of eliciting AMMO1-like antibodies could prevent oral transmission of the virus.

Here several protein subunit vaccines were generated where gH/gL is scaffolded onto self-assembling multimerization domains to produce nanoparticles with well-defined geometries and valency. Relative to monomeric gH/gL, immunization with the gH/gL nanoparticles elicited higher binding titers and neutralizing titers after one or two immunizations in mice. Competitive binding and depletion of plasma antibodies with an epitope-specific gH/gL probe suggested that only a small fraction of vaccine-elicited antibodies targeted the AMMO1 epitope. Consistent with this, depletion of plasma antibodies with an epitope-specific gH/gL knockout reduced plasma neutralizing activity to undetectable levels. Collectively these results demonstrate that gH/gL is an attractive vaccine antigen, but that multivalent display of gH/gL is required to elicit neutralizing antibodies of sufficient titer to protect against EBV infection.

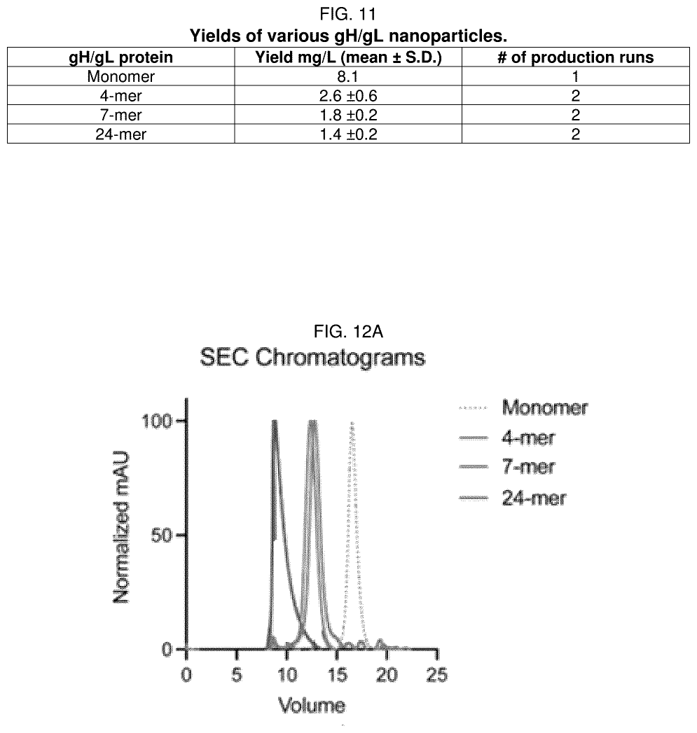

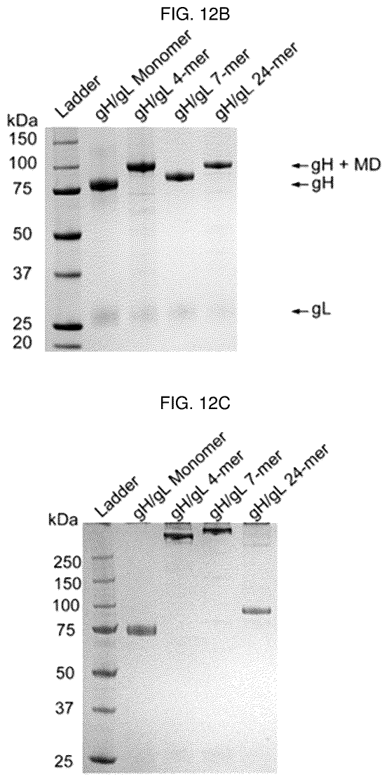

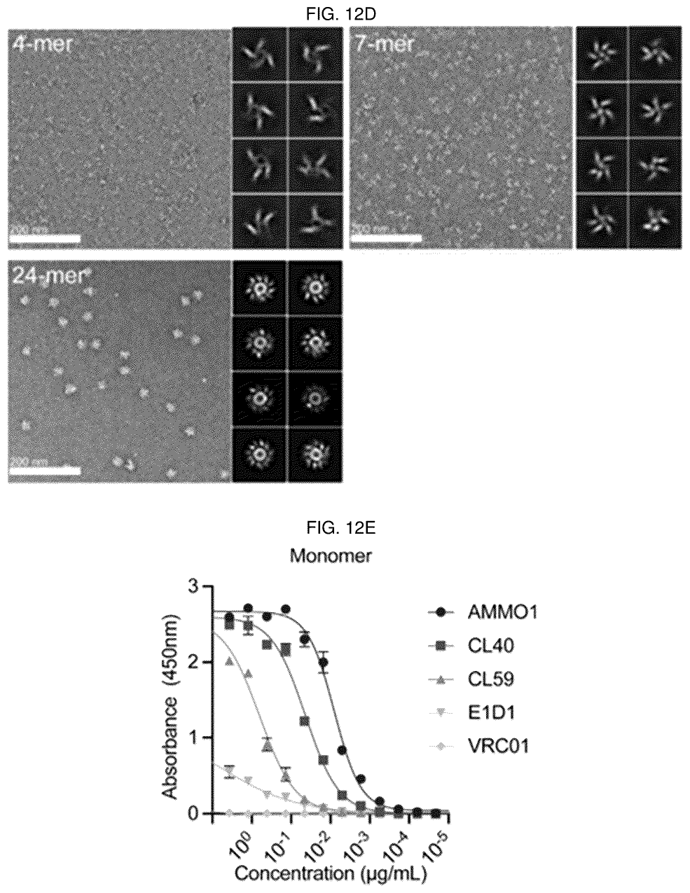

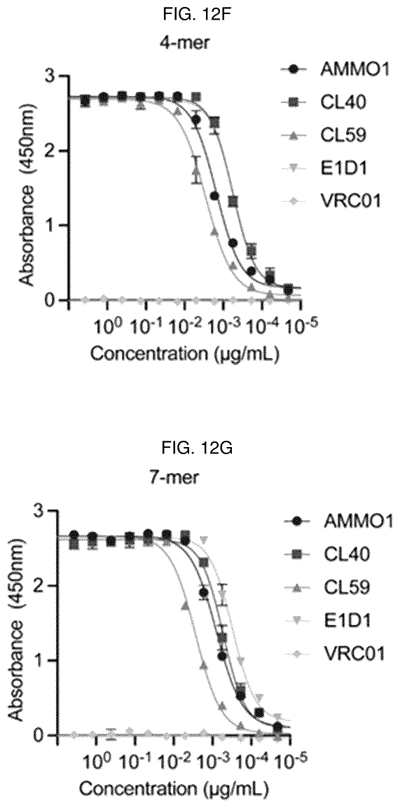

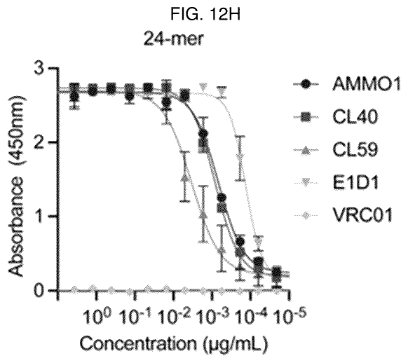

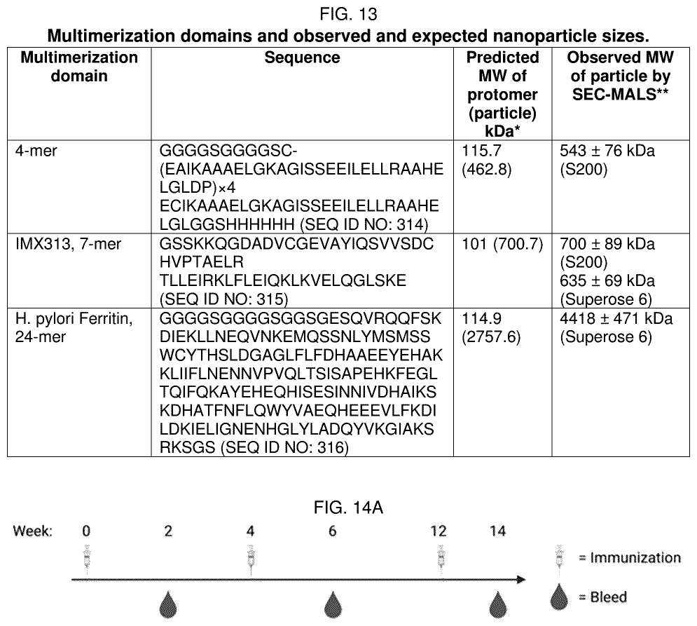

Results. Generation and Characterization of Multimeric gH/gL Vaccine Constructs. Cui et al. and Bu et al. have shown that immunization with multimeric gH/gL elicits higher serum neutralizing titers against infection of B cells and epithelial cells than immunization with monomeric gH/gL (Bu, W et al. (2019). Immunity 50, 1305-1316.e1306; and Cui, X., et al. (2016). Vaccine 34, 4050-4055). However, these studies focused on a single multimerization platform when generating gH/gL constructs, either Helicobacter pylori ferritin, a 24-mer, or a T4 fibritin foldon domain, a trimer. Several self-assembling multimeric gH/gL constructs with differing valencies, sizes, and geometries were developed to evaluate how they differ in their ability to elicit neutralizing antibodies in mice. Various expression constructs were generated where different multimerization domains were genetically fused to the C terminus of the gH ectodomain. These included i) a computationally designed circular tandem repeat protein (cTRP) that forms a planar toroid displaying four copies of gH/gL that is stabilized by inter-protomer disulfide bonds (Correnti, C. E., et al. (2020). Nat Struct Mol Biol 27, 342-350); ii) a modified version of the multimerization domain from the C4b-binding protein from Gallus gallus (IMX313) which also forms a planar, ring-like structure stabilized by inter-protomer disulfide bonds capable of displaying seven copies of gH/gL (Ogun, S. A., et al. (2008). Infection and immunity 76, 3817-3823); and iii) Helicobacter pylori ferritin which assembles into a 24-mer nanoparticle with octahedral symmetry and has previously been used to multimerize the EBV gp350 and gH/gL proteins(Bu, W et al. (2019). Immunity 50, 1305-1316.e1306; and Kanekiyo, M., et al. (2015) Cell 162, 1090-1100). The gH fusion proteins were co-expressed with gL using the Daedalus lentiviral expression system in HEK293 cells (Bandaranayake, A. D., et al. (2011). Nucleic Acids Res 39, e143). The gH/gL fusion proteins were purified by affinity chromatography followed by size-exclusion chromatography (SEC). The average yields, in mg/L of each purified gH/gL protein are provided in FIG. 11. The SEC elution profiles of the gH/gL fusion proteins were consistent with their expected size (FIGS. 12 and 13). The 4-mer and 7-mer constructs eluted earlier than the monomer. The gH/gL 24-mer eluted near the void volume. SEC-MALS revealed that the molecular weight of the particles were 540, 670, and 4420 for the 4-mer, 7-mer, and 24-mer, respectively, which are close to their predicted nanoparticle sizes (FIG. 13). Bands corresponding to the expected sizes of the gH fusion proteins were identified by reducing SDS-PAGE (FIG. 12B). Non-reducing SDS-PAGE revealed higher molecular weight complexes of the 4-mer and 7-mer consistent with the formation of inter-protomer disulfide bonds between the multimerization domain subunits (FIG. 12C). These analyses also revealed a band corresponding to gL and demonstrated that the preparations were highly pure (FIGS. 12B and 12C).

The gH/gL nanoparticles were imaged using negative-stain electron microscopy (nsEM), which demonstrated that all particles were monodisperse and of the predicted size. Density corresponding to gH/gL emanating from the nanoparticle cores was apparent in 2D class averages of the 4-mer, 7-mer and 24-mer (FIG. 12D).

To ensure that fusion to the multimerization domains did not alter the antigenicity of gH/gL, the binding of several anti-gH/gL mAbs to each nanoparticle were measured using an ELISA assay where biotinylated monomeric or gH/gL nanoparticles were captured on an ELISA plate coated with streptavidin. Of all the mAbs, AMMO1 binds with the highest affinity to monomeric gH/gL (FIG. 12E). The AMMO1 epitope bridges Domain I and Domain-II (D-I/D-II ) and spans both gH and gL(Snijder, J., et al. (2018). Immunity 48, 799-811.e799). CL40 has the second highest affinity (FIG. 12E) (Snijder, J., et al. (2018). Immunity 48, 799-811.e799), and binds to an epitope spanning the D-II/D-III interface of gH. CL59 binds at the C terminus of gH on D-IV (Sathiyamoorthy, K., et al. (2017). PNAS 114, E8703-E8710) and has lower affinity than CL40 or AMMO1 (FIG. 12E) (Snijder, J., et al. (2018). Immunity 48, 799-811.e799). E1D1 binds exclusively to gL and has the lowest affinity for the complex (FIG. 12E) (Snijder, J., et al. (2018). Immunity 48, 799-811.e799; and Sathiyamoorthy, K., et al. (2016). Nature communications 7, 13557).

In general, the mAbs maintained antigenicity to each multimeric construct, and some showed significant improvements in binding to the nanoparticles (FIGS. 12E-12H). Despite showing the weakest binding of all the mAbs to the gH/gL monomer, E1D1 showed the strongest binding to the 7-mer and the 24-mer (FIGS. 12G and 12H). The E1D1 epitope is most distal to the multimerization domains and is therefore highly exposed on the nanoparticles. Moreover, the spacing of the E1D1 epitope may be optimally presented for bivalent engagement by the E1D1 mAb in some formats. In contrast, CL59 showed the weakest binding to all the gH/gL nanoparticles. CL59 binds closer to the C terminus of the gH ectodomain, which would be in close proximity to the nanoparticle core, potentially limiting exposure of the epitope (FIGS. 12F-12H).

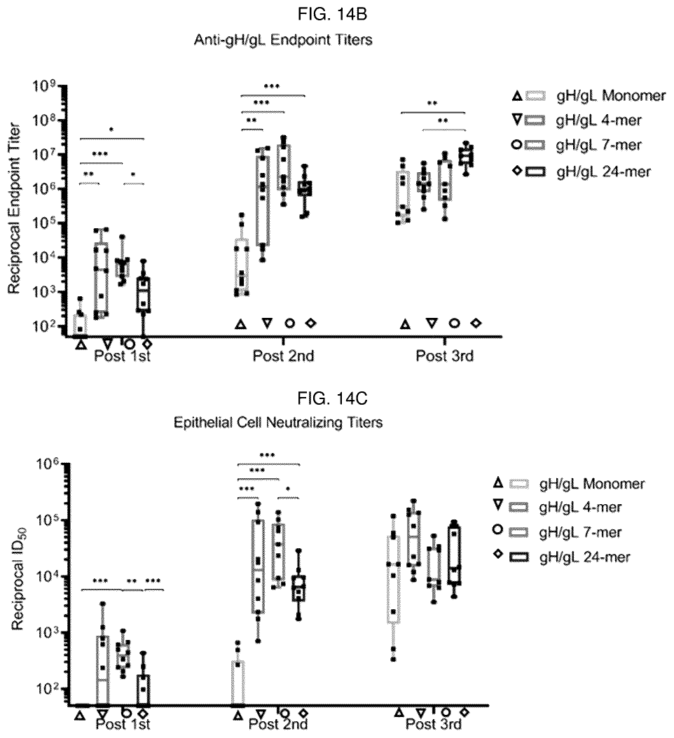

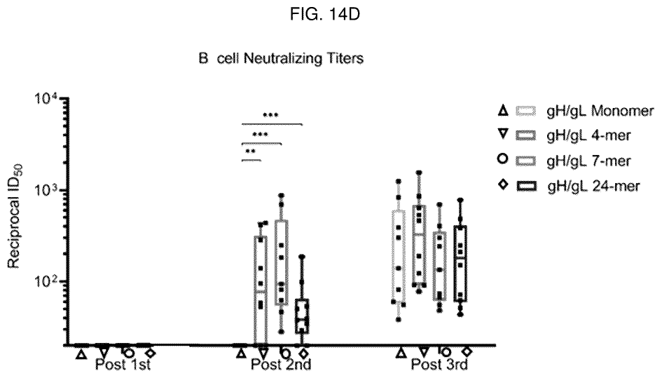

Immunogenicity of gH/gL nanoparticles. To assess the immunogenicity of the gH/gL nanoparticles, C57BL/6J mice were immunized with 5 μg of gH/gL monomer, 4-mer, 7-mer, or 24-mer formulated with adjuvant at weeks 0, 4 and 12. Plasma was collected two weeks post each immunization (FIG. 14A). Endpoint binding titers to gH/gL were measured by ELISA (FIG. 14B). After the first immunization, the median reciprocal binding titers in the gH/gL 4-mer, 7-mer, and 24-mer groups were higher than those in the monomer group. A second immunization boosted the binding titers in each group 200- to 1000-fold. Again, the median titers in animals immunized with the gH/gL 4-mer, 7-mer, and 24-mer were higher than in those immunized with monomeric gH/gL.

A third immunization with the monomer boosted the gH/gL binding titers such that they were comparable to those elicited by the 4-mer and 7-mer. A third immunization with the 24-mer also boosted the titers such that they were higher than the monomer and 4-mer groups, while the third immunization with the other nanoparticles did not further boost the median binding titers (FIG. 14B).

The ability of vaccine-elicited plasma to neutralize EBV infection of both B cells and epithelial cells was measured. To monitor neutralization in epithelial cells, the SVKCR2 cell line was used that stably expresses CR2 which promotes cellular attachment of virions via gp350 improving the otherwise poor infectivity of epithelial cells in vitro (Li, Q. X., et al. (1992). Nature 356, 347-350). Neutralizing activity against epithelial cell infection was elicited two weeks after the first immunization in all groups that received multimeric, but not monomeric gH/gL. The median titers were significantly higher in the 7-mer group compared to the monomer and 24-mer groups.

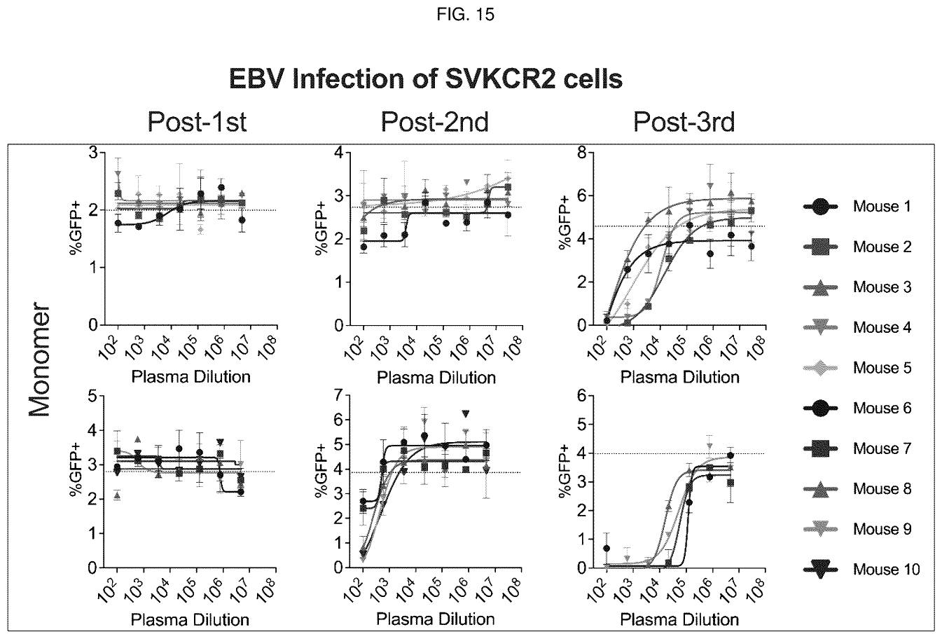

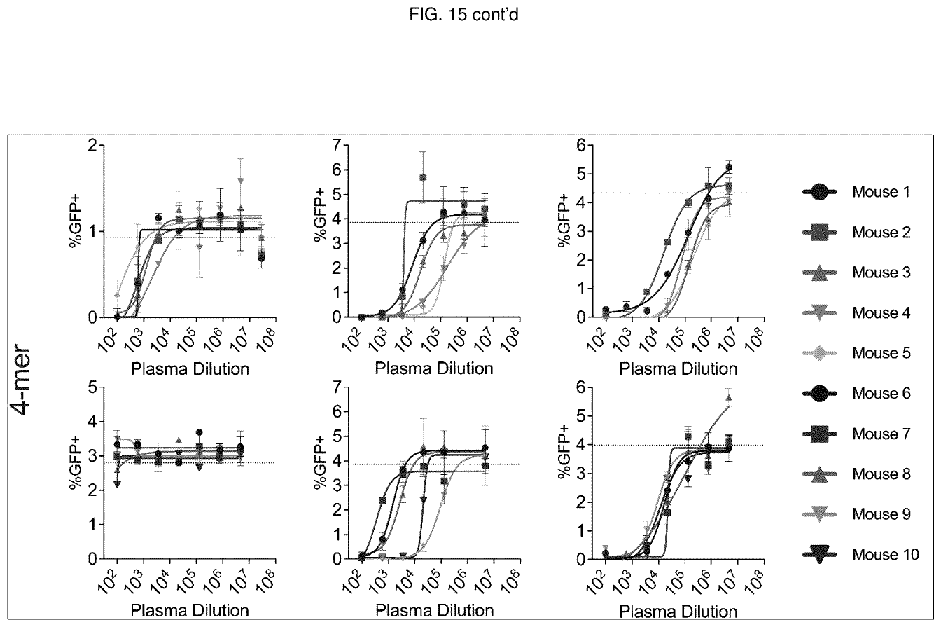

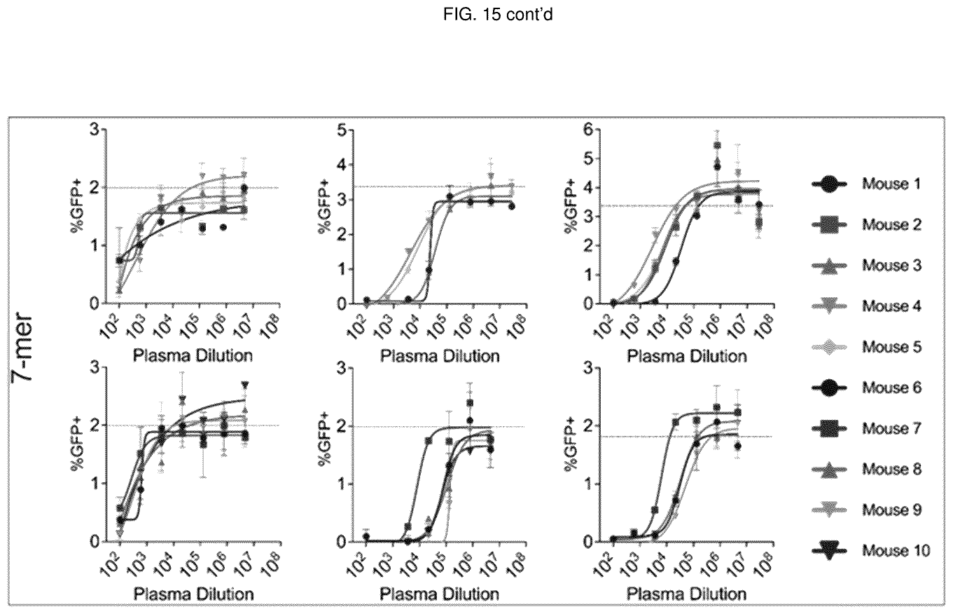

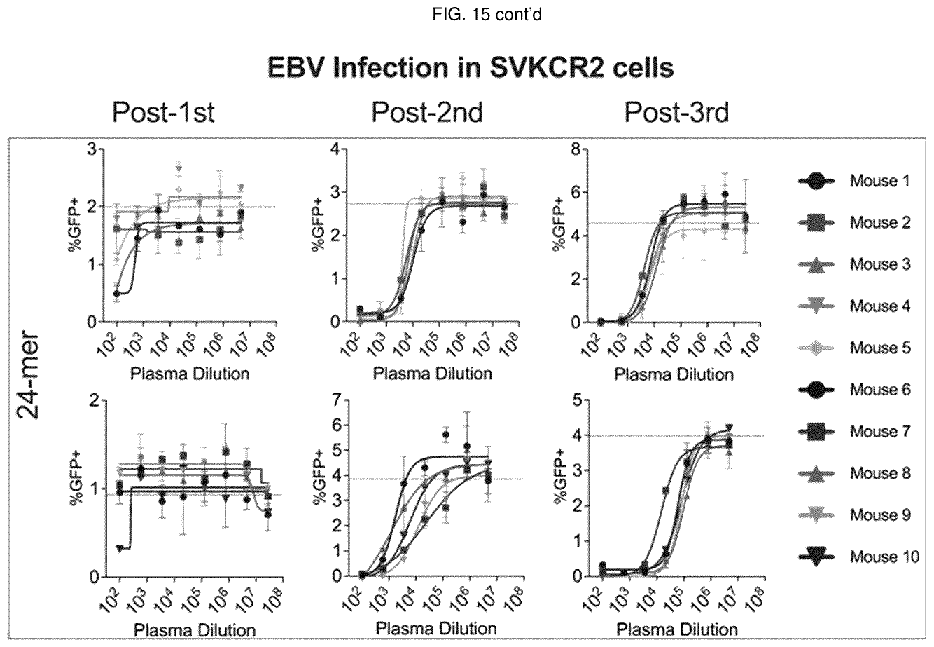

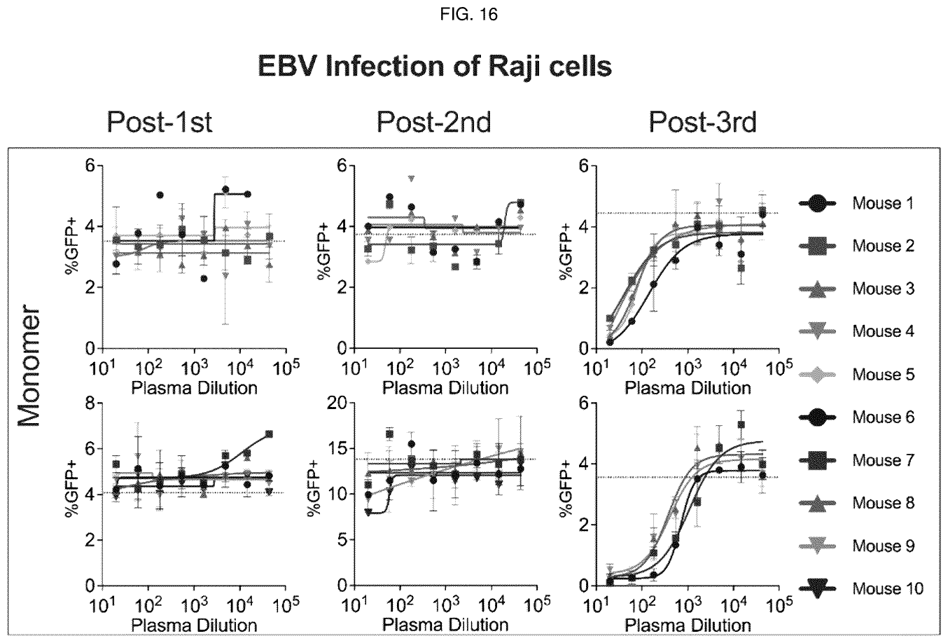

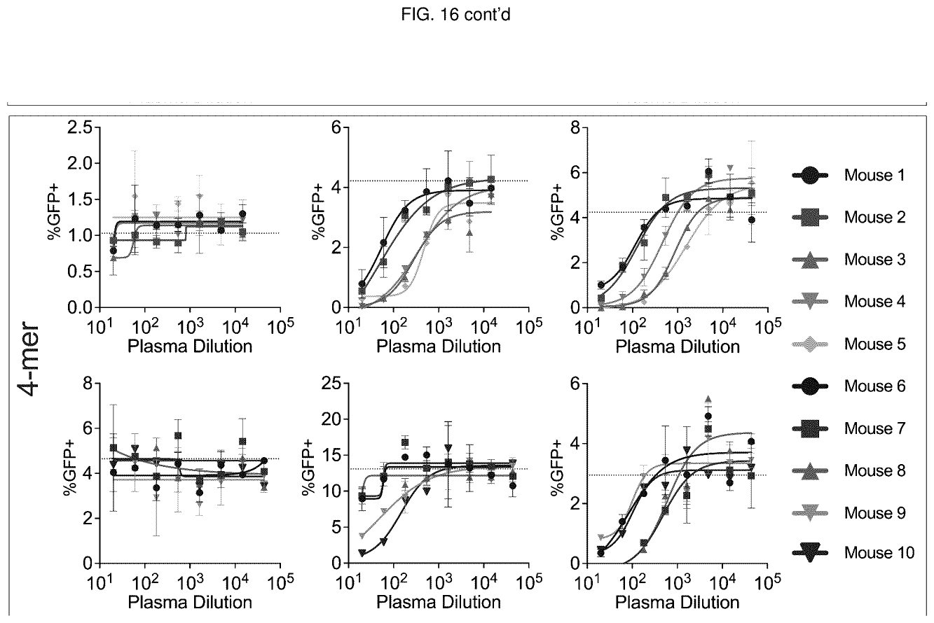

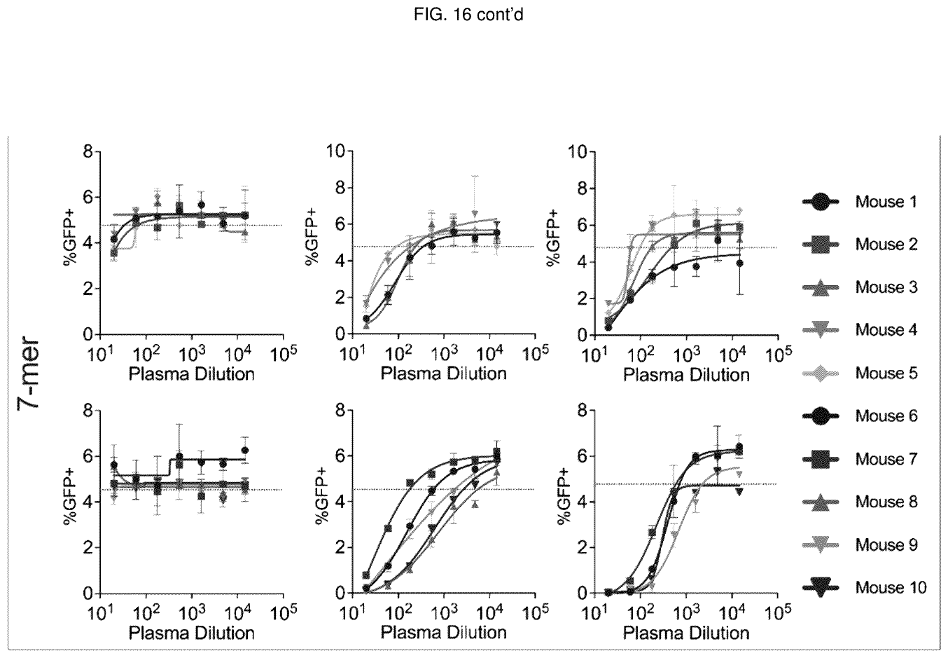

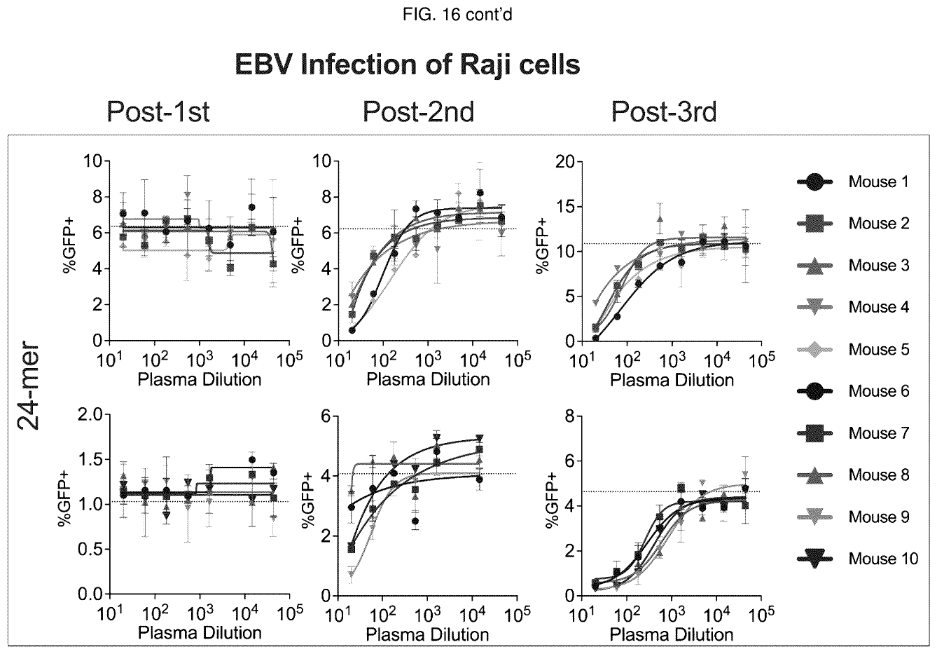

The second immunization boosted median neutralizing titers by -10-100 fold in the epithelial cell infection assay. The median neutralizing titers were higher in all of the gH/gL nanoparticle immunized groups than they were in the monomer group (FIGS. 14C and 15). The epithelial cell neutralizing titers in the 7-mer were also higher than those elicited by the 24-mer. The third immunization with the gH/gL nanoparticles did not further boost epithelial cell neutralizing responses, while the third dose of monomeric gH/gL boosted titers to levels that were comparable to those in other groups.

None of the gH/gL antigens elicited antibodies that could neutralize B cell infection two weeks after the first immunization (FIGS. 14D and S16. Following the second immunization, neutralizing titers were present in plasma from all groups immunized with gH/gL nanoparticles, but not in animals immunized with the monomer.

As was observed with the epithelial cell neutralizing titers, a third immunization with the gH/gL nanoparticles did not further boost B cell neutralizing responses, while a third dose of monomeric gH/gL boosted titers to levels that were comparable to those in other groups. In general, the neutralizing titers were 10-fold lower against B cell infection compared to epithelial cell infection in all groups. From these analyses, it was determined that all gH/gL nanoparticles displayed superior immunogenicity compared to monomeric gH/gL after one or two immunizations and that a third immunization did not result in a significant titer boost.

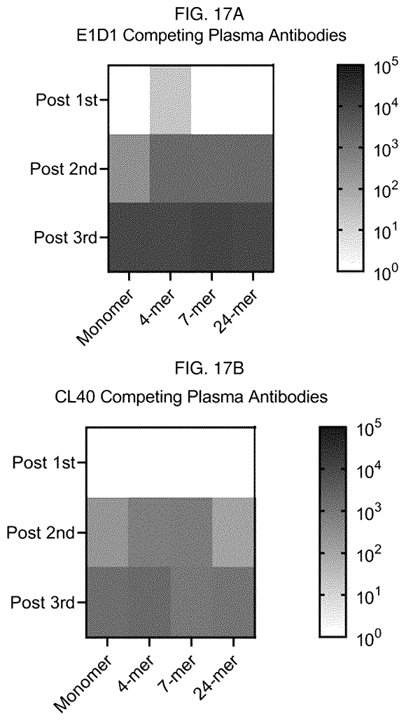

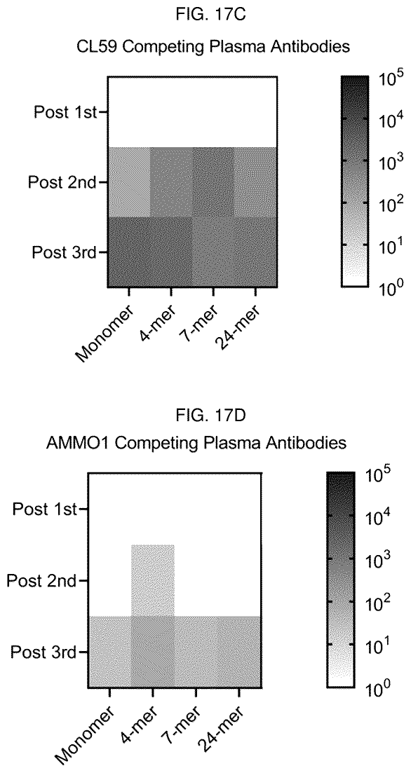

Plasma epitope mapping. Each multimeric gH/gL nanoparticle tested here has a unique valency and geometry which differentially affects the exposure of certain epitopes bound by neutralizing anti-gH/gL mAbs (FIGS. 12D-12H). To test whether the nanoparticle format skewed the epitope-specificity of vaccine-elicited antibodies from each construct, the ability of pooled immune plasma to compete with the E1D1, CL40, CL59 and AMMO1 mAbs was assessed for binding to monomeric gH/gL by ELISA (FIGS. 17A-17D).

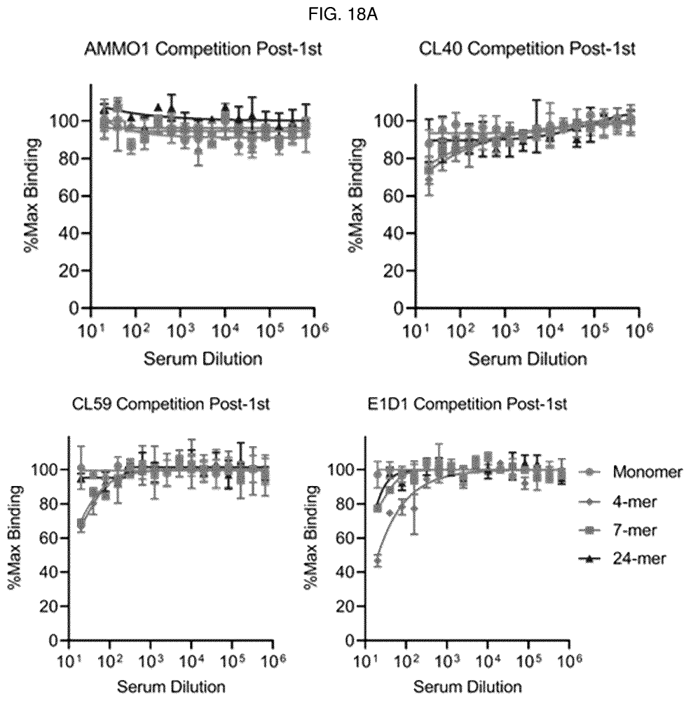

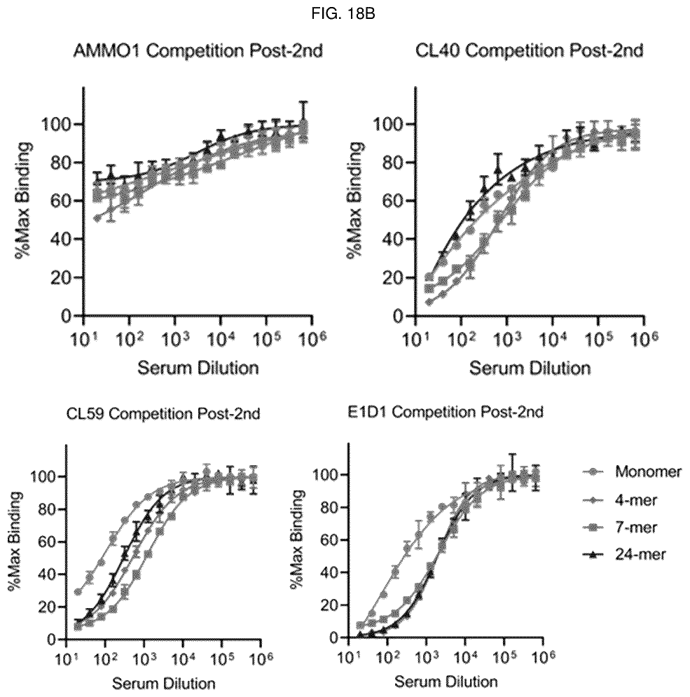

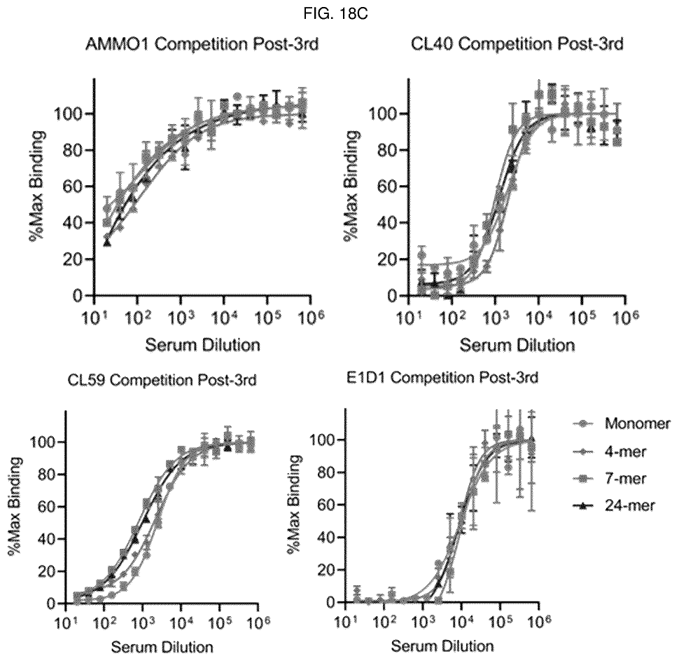

Pooled plasma collected following one immunization with the gH/gL 4-mer weakly inhibited E1D1 binding (FIGS. 17A and 18A). After the second immunization, plasma from all groups inhibited CL40, CL59, and E1D1 binding (FIGS. 17A-17C and 18B). Plasma antibodies that inhibited binding of these mAbs were further boosted following a third immunization in most groups. The only exception was that a third immunization with the 7-mer did not boost CL59-blocking plasma antibodies (FIGS. 17C and 18C).

Plasma antibodies capable of inhibiting AMMO1 binding were less common. Immune plasmas from the 4-mer group weakly inhibited AMMO1 binding after two immunizations, and were boosted following a third immunization (FIGS. 17D and 18C). All antigens elicited low titers of AMMO1-blocking antibodies following three immunizations.

These experiments demonstrate that each gH/gL nanoparticle readily elicits antibodies that compete with E1D1, and that AMMO1-competing antibodies are rarer. This difference in competition could be attributed to the relative affinities of these mAbs for gH/gL (FIG. 12E), or it could be due to the relative exposure of these epitopes on the nanoparticle.

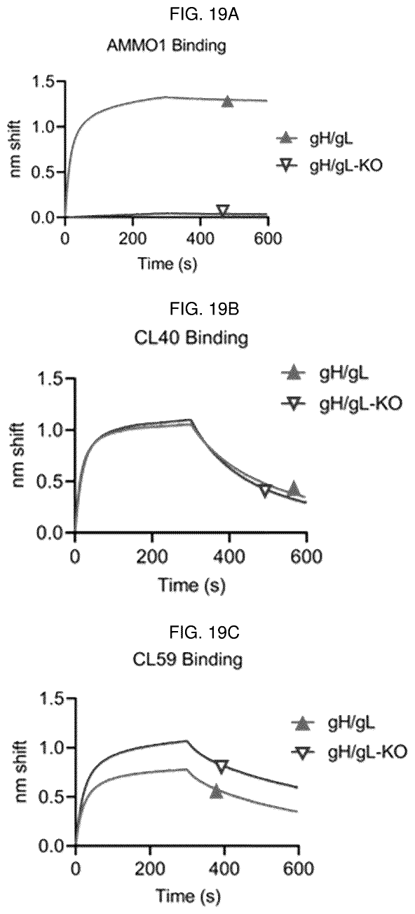

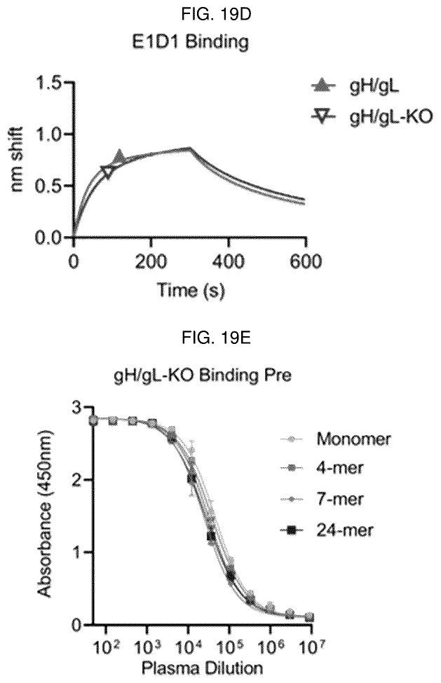

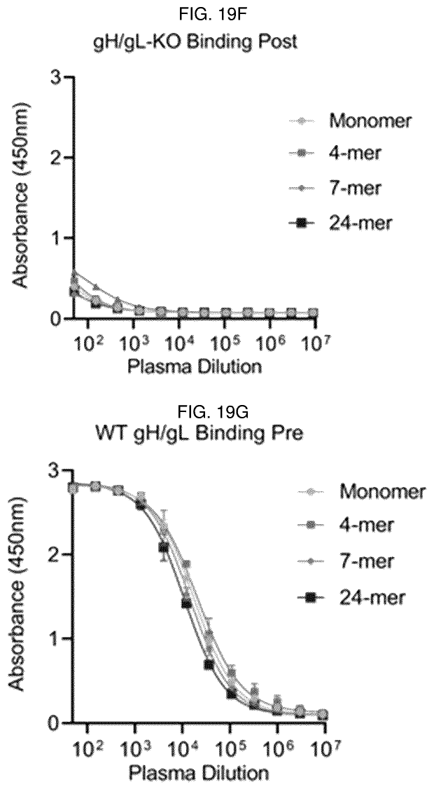

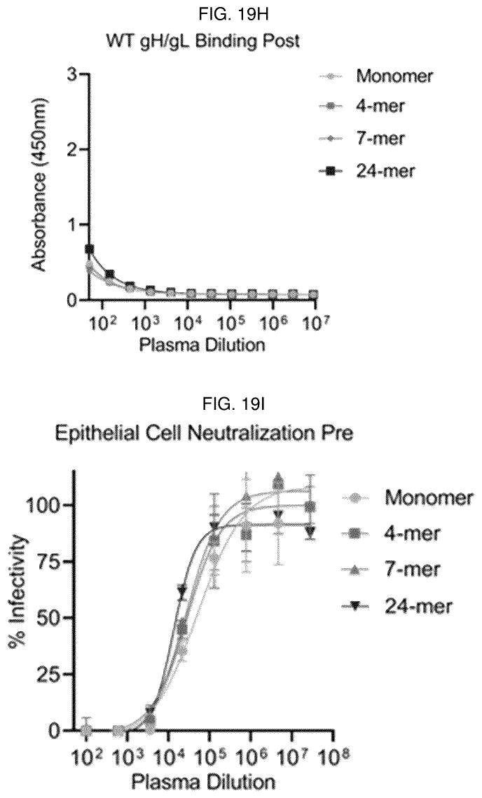

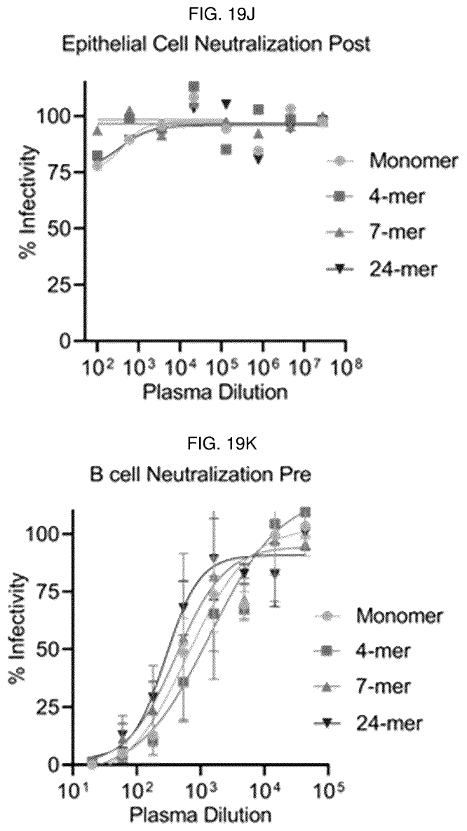

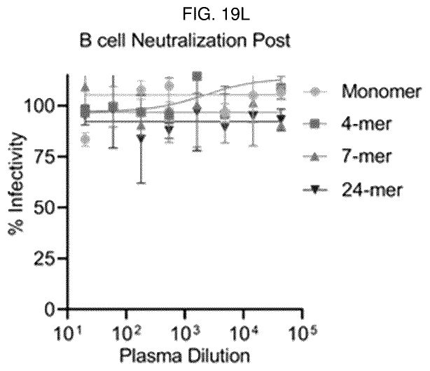

Although the titers of AMMO1-competing antibodies in the plasma of mice immunized with gH/gL nanoparticles are low, because the epitope bound by this mAb represents a critical site of vulnerability on gH/gL, the relative contribution of AMMO1-like antibodies to the plasma neutralizing activity of immunized mice was assessed. To achieve this, an epitope-specific gH/gL probe was developed and plasma depletions were carried out. Two mutations were previously identified, K73W and Y76A that reduced binding of AMMO1 to cell-surface expressed gH/gL (Snijder, J., et al. (2018). Immunity 48, 799-811.e799). A monomeric gH/gL ectodomain harboring these two mutations (herein called gH/gL-KO) which completely ablated AMMO1 binding while maintaining binding to other gH/gL mAbs as measured by biolayer interferometry (BLI) (FIGS. 19A-19D) were expressed and purified.

Antibodies from pooled plasma collected from each group two weeks after the third immunization were depleted using immobilized gH/gL-KO. ELISA binding of depleted plasma to gH/gL-KO confirmed depletion of gH/gL-KO-specific antibodies (FIGS. 19A-19L, compare panels E and F). Depletion with gH/gL-KO also reduced binding to wild-type gH/gL (FIGS. 19G and 19H). The binding signal was slightly stronger for gH/gL relative to gH/gL-KO post-depletion (FIGS. 19H and 19F), suggesting that very few plasma antibodies are sensitive to the KO mutations in the serum. Although the presence of antibodies that share the AMMO1 binding footprint but are insensitive to the KO mutations in this assay cannot be completely ruled out, these results are consistent with the mAb competition studies which demonstrated that there are very few AMMO1-like antibodies in the plasma of immunized animals (FIG. 17D and FIGS. 17A-17D).

Depletion of gH/gL-KO-specific antibodies led to a complete loss of neutralizing titers in both the B cell and epithelial neutralization assays (FIGS. 19I-19L). Collectively these data demonstrate that only a small portion of vaccine-elicited antibodies in each group target the AMMO1 epitope, and that they do not make a measurable contribution to the plasma neutralizing activity.

Discussion. A safe and effective vaccine could alleviate the global disease burden resulting from EBV infection. Here several multimeric vaccine candidates derived from the gH/gL ectodomain were developed and their ability to elicit antibodies capable of neutralizing EBV infection of both B cells and epithelial cells was evaluated in mice

Antigen multimerization has been used to improve the immunogenicity of subunit vaccines against several pathogens including malaria, HIV-1, RSV, SARS-CoV-2, influenza (Kanekiyo, M., et al. (2019). Nature immunology 20, 362-372; Walls, A. C., et al. (2021). Cell 184, 5432-5447. e5416; Boyoglu-Barnum, S., et al. (2021) Nature 592, 623-628; Marcandalli, J., et al. (2019). Cell 176, 1420-1431.e1417; Brouwer, P. J. M., et al. (2019). Nat Commun 10, 4272; Jardine, J., et al. (2013). Science 340, 711-716; Jelínková, L., et al. (2021). NPJ Vaccines 6, 13; and Moon, J. J., et al. (2012). Proc Natl Acad Sci USA 109, 1080-1085) and EBV (Bu, W et al. (2019). Immunity 50, 1305-1316.e1306; Cui, X., et al. (2016). Vaccine 34, 4050-4055; and Cui, X., et al. (2021). Vaccines 9, 285). Multimerization can enhance the immunogenicity of subunit vaccines through several mechanisms including more efficient B cell receptor cross linking, triggering of innate B cell responses, lymph node trafficking, and enhanced MHC class II antigen presentation (Bachmann, M. F., and Jennings, G. T. (2010). Nat Rev Immunol 10, 787-796; Irvine, D. J., et al. (2013). Nat Mater 12, 978-990; and Irvine, D. J., and Read, B. J. (2020). Curr Opin Immunol 65, 1-6). In general, mAb binding as measured by ELISA was generally improved by multimerization (FIGS. 12E-12H). Nevertheless, nanoparticle display resulted in a significant improvement in immunogenicity in vivo (FIGS. 14A-14D).

Previous studies have shown that antigen valency correlates with B cell activation, germinal center recruitment, and B cell differentiation as well as serum binding and neutralizing titers (Marcandalli, J., et al. (2019). Cell 176, 1420-1431.e1417; Veneziano, R., et al. (2020). Nat Nanotechnol 15, 716-723; and Kato, Y., et al. (2020). Immunity 53, 548-563.e548). Although nanoparticles displaying gH/gL exhibited superior immunogenicity as compared to monomeric gH/gL, a strict correlation between antigen valency and binding or neutralizing titers was not observed. The differences in the ability of these antigens to elicit neutralizing antibodies could be linked to nanoparticle stability in vivo or T cell help directed at MHCII-restricted epitopes that differ between the nanoparticle scaffolds (Arunachalam, P. S., et al. (2021). Nature 594, 253-258).

In sum, multivalent display of EBV gH/gL markedly enhanced immunogenicity in mice. These results underscore the importance that vaccine-elicited antibodies against gH/gL can play in preventing EBV infection and highlight the utility of cutting-edge vaccine approaches in the development of vaccines against this important cancer-associated pathogen.

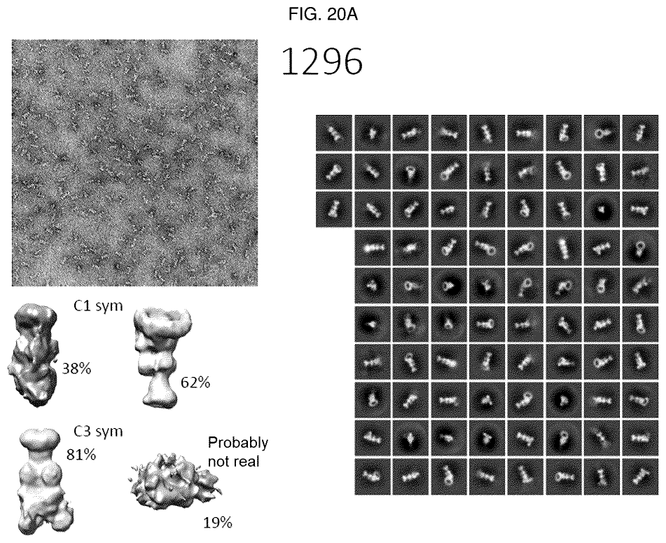

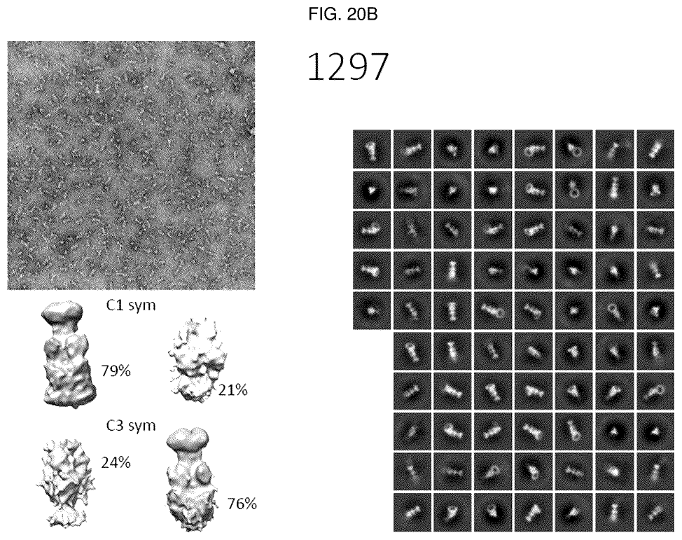

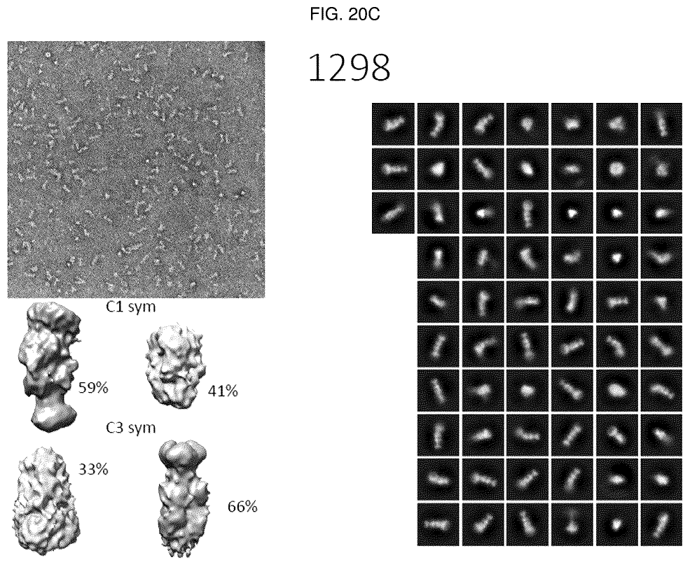

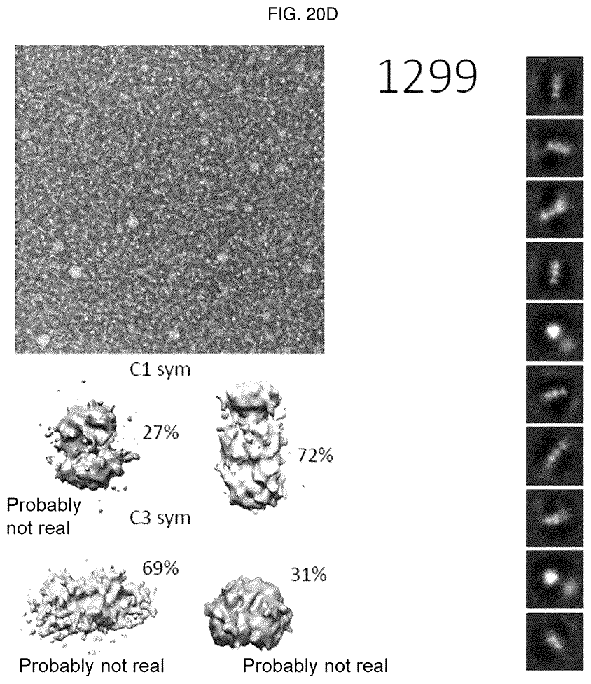

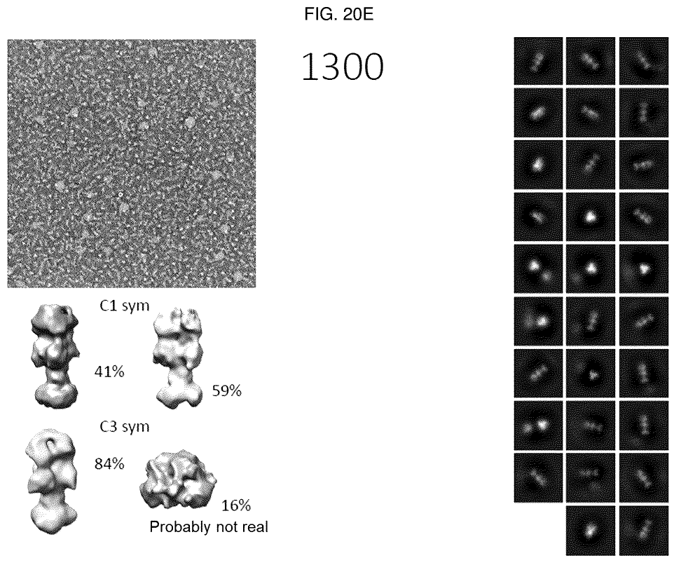

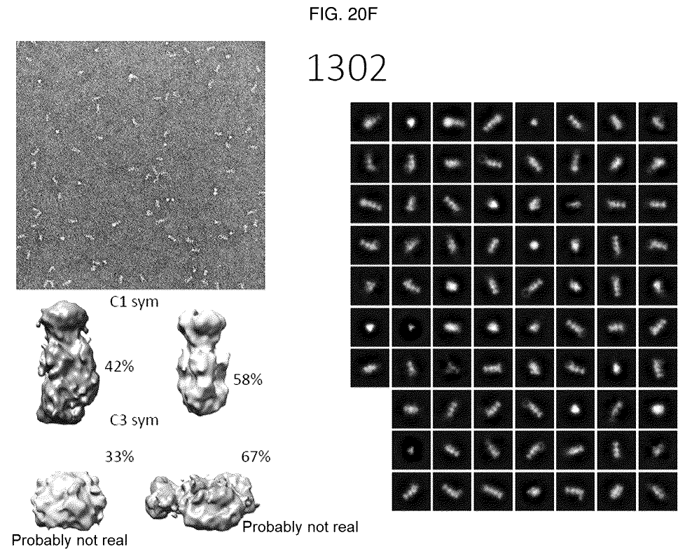

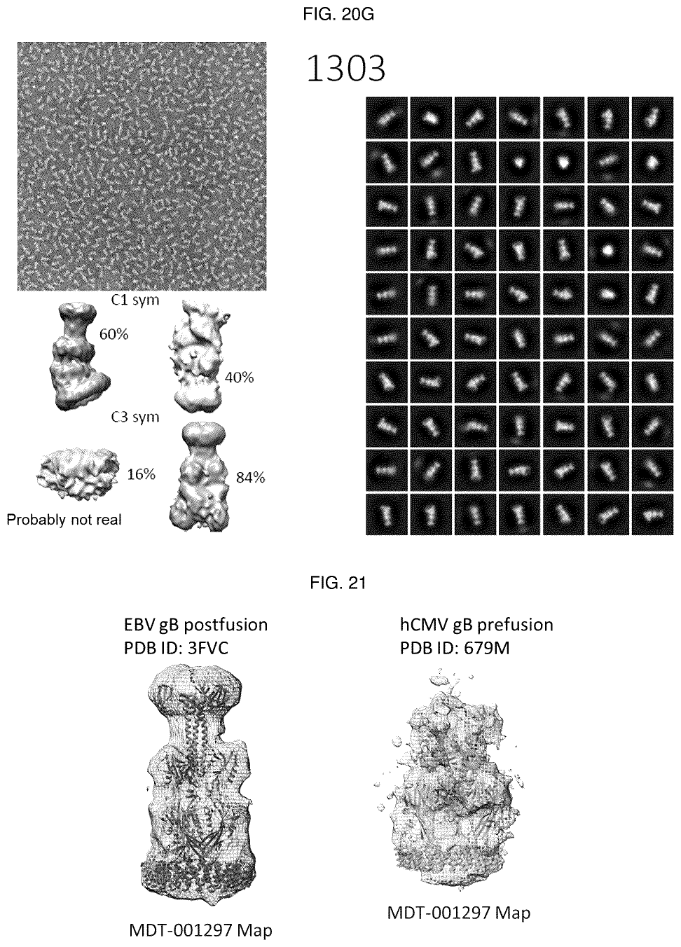

Example 2. Nine EBV gB-cTRP fusions were produced by varying the C-terminal truncation site on gB and the length of the GS-based linker connecting gB to the cTRP scaffold with the goal that some variants would best stabilize the meta-stable prefusion state of gB. Purified products for each of the variants was analyzed by negative stain electron microscopy (EM) and the data is in FIGS. 20A-20G. Images of the negative stain EM grids are displayed as well as zoomed in images of individual molecules from the grid selected for 3D reconstruction. 3D reconstruction was performed imparting either C1 or C3 symmetry and the resulting maps are presented for each variant where data was collected. The aim was to identify gB-cTRP fusions that favored adoption of the “shorter and squatter” prefusion state. Published 3D models of the related hCMV prefusion structure (PDB ID: 679M) and EBV gB postfusion structure (PDB ID: 3FVC) were fit into our experimentally determined EM maps and gB-cTRP variants were identified where the hCMV prefusion model structure appeared to fit well. All variants appeared to have postfusion-like structures exclusively, except for MDT-001297 and MDT-001298 which appeared to have a fraction of the imaged molecules in a prefusion-like state. The models fit into the EM maps for MDT-001297 are shown in FIG. 21. From this data set, it can be concluded that the C-terminal truncation site and fusion site linker length are critical for stabilization of the desired EBV gB prefusion state and that iterations based upon MDT-001297 represent promising EBV vaccine candidates.

(xi) Closing Paragraphs. As indicated previously, variants of the sequences disclosed and referenced herein are included. In particular embodiments, variants of proteins can include those having one or more conservative amino acid substitutions or one or more non-conservative substitutions that do not adversely affect the function of the protein in a relevant physiological measure. A “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gin); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

In particular embodiments, variants of the protein sequences (e.g., vaccine proteins, and/or cTRP scaffolds) disclosed herein include sequences with at least 70% sequence identity, at least 80%, at least 85%, at least 90%, at least 95%, at least 96%, at least 97%, at least 98%, or at least 99% sequence identity to the protein sequences described or disclosed herein.

“% sequence identity” refers to a relationship between two or more sequences, as determined by comparing the sequences. In the art, “identity” also means the degree of sequence relatedness between protein sequences or nucleic acid sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine % sequence identity are designed to give the best match between the sequences tested. Methods to determine % sequence identity and similarity are codified in publicly available computer programs. Sequence alignments and % sequence identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, NY. Within the context of this disclosure it will be understood that where sequence analysis software is used for analysis, the results of the analysis are based on the “default values” of the program referenced. “Default values” will mean any set of values or parameters, which originally load with the software when first initialized.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, a material effect would cause a statistically-significant reduction in a primary antibody response to an EBV vaccine.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. An EBV vaccine comprising the sequence as set forth in SEQ ID NOs: 304, 305, 306, 307, 308, 309, 310, 311, 312, or 313 or a sequence having at least 90% sequence identity to the sequence as set forth in SEQ ID NOs: 304, 305, 306, 307, 308, 309, 310, 311, 312, or 313.

2. The EBV vaccine of claim 1 comprising the sequence as set forth in SEQ ID NO: 307 or SEQ ID NO: 308 or a sequence having at least 90% sequence identity to the sequence as set forth in SEQ ID NO: 307 or SEQ ID NO: 308.

3. The EBV vaccine of claim 1, comprising the sequence as set forth in SEQ ID NOs: 294, 295, 296, 297, 298, 299, 300, 301, 302, or 303 or a sequence having at least 90% sequence identity to the sequence as set forth in SEQ ID NOs: 294, 295, 296, 297, 298, 299, 300, 301, 302, or 303.

4. The EBV vaccine of claim 1, comprising the sequence as set forth in SEQ ID NO: 297 or SEQ ID NO: 298 or a sequence having at least 90% sequence identity to the sequence as set forth in SEQ ID NO: 297 or SEQ ID NO: 298.

5. The EBV vaccine of claim 1, comprising the sequence as set forth in SEQ ID NOs: 6, 7, 8, 9, 10, 11, 12, 13, 14, or 15 or a sequence having at least 90% sequence identity to the sequence as set forth in SEQ ID NOs: 6, 7, 8, 9, 10, 11, 12, 13, 14, or 15.

6-44. (canceled)

45. A nucleotide sequence encoding an EBV vaccine of claim 1.

46-49. (canceled)

50. A method of stimulating an anti-EBV immune response in a subject comprising administering to the subject a therapeutically effective amount of the EBV vaccine of claim 1 to the subject, thereby stimulating an EBV immune response in the subject.

51. The method of claim 50, wherein the subject is EBV seropositive.

52. The method of claim 50, wherein the subject is an EBV seronegative subject, and wherein the therapeutically effective amount reduces the risk of EBV infection.

53. The method of claim 52, wherein the EBV seronegative subject is a pediatric patient.

54. The method of claim 50, wherein the administering is prior to treatment with an immunosuppressant.

55. The method of claim 50, wherein the subject is a transplant patient.

56. The method of claim 50, wherein the therapeutically effective amount reduces the risk or severity of infectious mononucleosis (IM).

57. The method of claim 50, wherein the method treats the subject for an EBV infection.

58. The method of claim 57, wherein the treating reduces or eliminates infectious mononucleosis (IM) and/or symptoms of IM.

59. The method of claim 57, wherein the treating (i) reduces or eliminates a lymphoproliferative disorder and/or symptoms of the lymphoproliferative disorder, (ii) reduces the risk or occurrence of a carcinoma, and/or (iii) reduces the risk or occurrence of a smooth muscle tumor.

60. The method of claim 59, wherein lymphoproliferative disorder comprises Hodgkin lymphoma, non-Hodgkin lymphoma, Burkitt lymphoma, T cell lymphoma, or lymphoproliferative disease.

61. The method of claim 60, wherein the carcinoma comprises nasopharyngeal carcinoma or gastric carcinoma.

62. The method of claim 50, further comprising administering a second therapeutically effective amount of the EBV vaccine of claim 1 to the subject.

63. The method of claim 50, further comprising administering a third therapeutically effective amount of the EBV vaccine of claim 1 to the subject.
